(19) United States
(12) Patent Application Publication
(10) Pub. No.: US 2019/0049436 A1
(43) Pub. Date: Feb. 14, 2019

(54) MODULATION OF ASYMMETRIC PROLIFERATION

(71) Applicant: THE GENERAL HOSPITAL CORPORATION, Boston, MA (US)

(72) Inventor: Sridhar RAMASWAMY, Wellesley, MA (US)

(73) Assignee: THE GENERAL HOSPITAL CORPORATION, Boston, MA (US)

(21) Appl. No.: 15/939,522

(22) Filed: Mar. 29, 2018

Related U.S. Application Data
(63) Continuation of application No. 14/603,866, filed on Jan. 23, 2015, now abandoned.
Application No. 14/603,866 is a continuation-in-part of application No. PCT/US2013/060842, filed on Sep. 20, 2013.
(60) Provisional application No. 61/704,033, filed on Sep. 21, 2012.
(60) Provisional application No. 62/101,510, filed on Jan. 9, 2015.

(51) Int. Cl. G01N 33/50; C12N 5/09

(52) U.S. Cl. CPC G01N 33/5091; G01N 2333/9015; C12N 5/0693

(57) ABSTRACT

Provided herein are methods and compositions related to modulation of the rate of asymmetric proliferation of cancer cells. In some embodiments, the methods described herein relate to the treatment of cancer, at least in part, via the modulation of the rate of asymmetric proliferation of cancer cells.

### CROSS-REFERENCE TO RELATED APPLICATIONS

This application is a continuation under 35 U.S.C. § 120 of co-pending U.S. patent application Ser. No. 14/603,866 filed Jan. 23, 2015, which claims benefit under 35 U.S.C. § 119(e) of U.S. Provisional Application No. 62/101,510 filed Jan. 9, 2015 and is a continuation-in-part application of International Patent Application No. PCT/US13/60842 filed Sep. 20, 2013, which designates the United States and which claims benefit under 35 U.S.C. § 119(e) of U.S. Provisional Application No. 61/704,033 filed Sep. 21, 2012, the contents of which are incorporated herein by reference in their entirety.

### GOVERNMENT SUPPORT

This invention was made with federal funding under Grant No. C06 CA059267 awarded by the National Cancer Institute. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Jan. 14, 2015, is named 030258-075393-US_SL.txt and is 782 bytes in size.

### TECHNICAL FIELD

The technology described herein relates to the modulation of asymmetric cell division and proliferation in cancer cells.

### BACKGROUND

Tumors can comprise both rapidly proliferating and slowly proliferating cells. Tumors comprising particularly rapidly proliferating cells clearly are capable of faster growth and progression. But these tumors also contain many slowly proliferating cancer cells that may complicate treatment by resisting cancer therapeutics which preferentially target fast proliferators. While clonal selection theory clearly explains how rapidly proliferating cancer cells evolve, it remains difficult to understand within this framework why even advanced tumors contain so many slowly proliferating cancer cells (P. C. Nowell, Science 194, 23 (Oct. 1, 1976)). In culture, cancer cells have been observed to occasionally divide in such a manner that one daughter cell will have a markedly slower proliferative rate than the other, a phenomenon referred to as “asymmetric proliferation.” The occurrence of asymmetric proliferation is generally assumed to simply reflect random variation among individual cancer cells in the many genetic and non-genetic factors that influence transit through the cell cycle (J. Massague, Nature 432, 298 (Nov. 18, 2004)).

### BRIEF DESCRIPTION OF THE DRAWINGS

FIGS. 1A-1P demonstrate that mTORC2-AKT1 signaling regulates asymmetric cancer cell division. FIG. 1A depicts a crystal structure of AKT1 protein with mutated sites noted. In FIGS. 1B-1E and 1G-1P, the bar graphs depict percentages of H3K9me2^{low}/MCM2^{low}/HES1^{high} asymmetric cells and G0-like cells. FIG. 1B depicts the results of AKT1/2^{-/-} HCT116 cells replaced with AKT1 and AKT2 cDNAs, HCT116 wild type (WT). FIG. 1C depicts the results of mutation of AKT, e.g. with AKT1-T308A, AKT1-S473A, AKT1-T308A/S473A and AKT1-T450A cDNAs, HCT116 WT. FIGS. 1D-1E depict the results of HCT116 and MCF7 cells, respectively treated with DMSO, control, TORIN1, AZD8055, INK128, Palomid 529, Rapamycin and RAD-001, for 72 h. FIGS. 1G-1K depict the results of RICTOR knockdown in HCT116 (FIG. 1G), MCF7 (FIG. 1H), MDA-MB-231 (FIG. 1I), PC9 (FIG. 1J) and A375 (FIG. 1K) cells with control, non-silencing(NS) hairpin(hp) and RICTOR knockdown hp1,4. FIGS. 1L-1M depict the results of HCT116 and MCF7 cells, respectively treated with DMSO, control and kinase inhibitors AZD5363, GDC0068, for 72 hours. FIG. 1N depicts the results of AKT1/2^{-/-} HCT116 cells replaced with AKT1-E17K cDNA, HCT116 WT. FIGS. 1O-1P depicts the results of HCT116 and MCF7 cells, respectively treated with DMSO, control and AKT allosteric inhibitors AKT1/2, MK2206, for 72 h. Error bars indicate mean±SEM. FIG. 1F depicts the results of a Western blot for RICTOR in HCT116 cells.

FIGS. 2A-2F demonstrate that a TTC3-proteasome pathway is necessary for G0-like cells. FIG. 2A depicts the results of a Western blot for TTC3 in HCT116 cells. FIGS. 2B-2F depict bar graphs of percentages of H3K9me2^{low}/MCM2^{low}/HES1^{high}. FIGS. 2B-2C depict G0-like cells in HCT116 (FIG. 2B) and MCF7 (FIG. 2C) cells with control, NS hp, and TTC3 knockdown hp3-5. FIG. 2D depicts the results of AKT1/2^{-/-} HCT116 cells replaced with AKT1-K8R, AKT1-K14R and AKT1-K8R/K14R cDNAs, HCT116 WT. FIGS. 2E-2F depict the results of HCT116 (FIG. 2E) and MCF7 (FIG. 2F) cells treated with DMSO, control, MG-132 and Bortezomib for 24 hours. Error bars indicate mean±SEM. Arrow indicates a G0-like TTC3⁺ cell.

FIG. 3 depicts a graph demonstrating that mTORC2 signaling induces slow proliferators. Percentage of sibling pairs with cell cycle times<t (βcurves) for HCT-116 cells transfected with either control, NS (lighter dots forming the right hand trend) vs. RICTOR knockdown, hp4 (darker dots forming the left-hand trend) shRNA. Each point is calculated at 20 minute intervals and only shown if there was at least one event occurring. N=701 and 1295 for total number of cells counted for NS and RICTOR knockdown groups, respectively.

FIGS. 4A-4N demonstrate that asymmetrically dividing cancer cells and slow proliferators promote tumorigenesis in vivo. FIGS. 4A-4E depict the results of control, NS (squares) and RICTOR knockdown shRNAs hp1,4 (triangles and diamonds, respectively) of HCT116 (FIG. 4A), MCF7 (FIG. 4B), MDA-MB-231 (FIG. 4C), PC9 (FIG. 4D) and A375 (FIG. 4E) cells were injected in Nu/Nu mice and their tumor growth followed over a number of days. FIGS. 4F-4J, 4L, and 4N demonstrate that tumor forming potential of cells treated with DMSO, control (squares) or AKT1/2 inhibitor for 72 h (diamonds) in (FIGS. 4F-4J) HCT116 and (FIGS. 4L and 4M) MCF7 cells, respectively. Serial dilutions of (FIGS. 4F,4L) 5×10⁶, (4G,4M) 5×10⁵, (4H) 5×10⁴, (4I) 5×10³, (4J) 5×10² cells were used to inject mice and the tumor formation and growth was monitored over several days. Error bars indicate mean±SEM. FIGS. 4K and 4N depict images of mice injected with (4K) 5×10⁵ HCT116 cells, (4N) 5×10⁶ MCF7 cells, C (control, DMSO treated), I (induced, AKT1/2 inhibitor treated).

FIG. 5 depicts a working model for asymmetric cancer cell division.

FIGS. 6A-6E demonstrate that RICTOR knockdown does not alter proliferation in vitro. Proliferation assay were performed over 5 days for control, NS shRNA (squares) and RICTOR knockdown shRNAs hp1,4 (diamonds and triangles, respectively) in (6A) HCT116, (6B) MCF7, (6C) MDA-MB-231, (6D) PC9 and (6E) A375 cell lines, under normal conditions. Error bars indicate mean±SEM.

FIGS. 7A-7J demonstrate that β1-integrin -FAK-mTORC2-AKT1 signaling regulates the production of slow proliferators. FIG. 7A depicts a schematic model of AKT1 protein. FIGS. 7B-7H depict graphical representation of percentage of change of H3K9me2low/MCM2low/HES1high asymmetrically dividing and G0-like cells compared to control in HCT116 and MCF7 cell lines. Error bars indicate mean±SEM. FIGS. 7I-7J depict plots for percentage of sibling pairs with cell cycle time difference<t. (K) HCT116 (L) MCF7 cells with control, NS (right-hand trend) or RICTOR, hp4 (left hand trend). N=701 and 1295 for total number of cells counted for NS and RICTOR knockdown groups, respectively.

FIG. 8 demonstrates the interaction of FAK with RICTOR. In HCT116 and MCF7 cells, RICTOR was immunoprecipitated with anti-FAK and immunoblotted with anti-mTOR, anti-RICTOR and anti-RAPTOR antibody. Reciprocally, FAK was immunoprecipitated with anti-RICTOR and immunoblotted with anti-mTOR and anti-FAK, in G1 as well as M phase of the cell cycle.

FIGS. 9A-9C demonstrate that slow proliferators promote tumorigenesis in vivo. FIG. 9A depicts the experimental procedure and results for mice with subcutaneous tumors treated with TS2/16 antibody once a week for 5 weeks or untreated (control). FIG. 9B depicts the experimental procedure and results when Inducible Non-Silencing shRNA (control) or RICTOR knockdown shRNAs hp1,4 of 5 different cell lines were injected into mice. FIG. 9C depicts the experimental procedure and results when serial dilutions of HCT116 and MCF7 cells incubated with DMSO (control) or AKT1/2i for 72hours were injected into mice. Tumor volume was followed weekly. Error bars indicate mean±SEM for five mice per group.

FIG. 10 depicts a working model for asymmetric cancer cell division.

FIGS. 11A-11C demonstrate knockdown of proteins in HCT116 cells. FIGS. 11A and 11B depict knockdown of FAK (FIG. 11A) and β1-integrin (FIG. 11B) in HCT116 cells with Non-Silencing (NS) as control shRNA. FIG. 11C depicts a graphical representation of percentage of change of H3K9me2low/MCM2low/HES1high asymmetrically dividing and G0-like cells compared to control in RICTOR knockdown cell lines. Error bars indicate mean±SEM.

FIGS. 12A-12O demonstrate that RICTOR knockdown does not alter proliferation in vitro. FIGS. 12A-12O depict graphs of the results of proliferation assay over 5 days for control, NS hp (squares) and RICTOR knockdown shRNAs hp1,4 (triangles and circles, respectively) in (FIG. 12A) HCT116, (FIG. 12B) MCF7, (FIG. 12C) MDA-MB-231, (FIG. 12D) PC9 and (FIG. 12E) A375 cell lines, under normal conditions; in (FIG. 12F) HCT116, (FIG. 12G) MCF7, (FIG. 12H) MDA-MB-231, (FIG. 12I) PC9 and (FIG. 12J) A375 cell lines, under hypoxia conditions; in (FIG. 12K) HCT116, (FIG. 12L) MCF7, (FIG. 12M) MDA-MB-231, (FIG. 12N) PC9 and (FIG. 12O) A375 cell lines, under low serum conditions. Error bars indicate mean±SEM.

FIGS. 13A-13J demonstrate that RICTOR knockdown does not alter proliferation in vitro. FIGS. 13A-13E depict graphs of proliferation assay over 5 days for control, NS hp (squares) and RICTOR knockdown shRNAs hp1,4 (triangles and circles, respectively) in (FIG. 13A) HCT116, (FIG. 13B) MCF7, (FIG. 13C) MDA-MB-231, (FIG. 13D) PC9 and (FIG. 13E) A375 cell lines, under low glucose conditions. FIGS. 13F-13J depict the results of clonogenic assay over 2 weeks after irradiation for control, NS hp and RICTOR knockdown shRNAs hp1,4 in (FIG. 13F) HCT116, (FIG. 13G) MCF7, (FIG. 13H) MDA-MB-231, (FIG. 13I) PC9 and (FIG. 13J) A375 cell lines. Error bars indicate mean±SEM.

FIGS. 14A-14D demonstrate that RICTOR knockdown does not alter invasion in vitro. FIGS. 14A-14D depict the results of invasion assay over 24 hours for control, NS hp (first bar) and RICTOR knockdown shRNAs hp1,4 (second and third bars, respectively) in (FIG. 14A) HCT116, (FIG. 14B) MCF7, (FIG. 14C) PC9 and (FIG. 14D) A375 cell lines. Error bars indicate mean±SEM.

FIGS. 15A-15D depict a mechanism for AKT1^{low} slow proliferators: AKT1, TTC3, and proteasome. FIG. 15A depicts a bar graph of percentages of H3K9me2low/MCM2low/HES1high asymmetric mitoses and G0-like cells in AKT1/2^{-/31} HCT116 cells with cDNAs for AKT1 or AKT2 or AKT1-K179M or AKT1-D292A. FIG. 15B depicts a schematic model of AKT1 protein with C, catalytic; P, phosphorylation; Ub, ubiquitination; PH, pleckstrin homology; HD, hydrophobic domain. FIG. 15C depicts a graphical representation of percentage change in H3K9me2low/MCM2low/HES1high asymmetrically dividing and G0-like cells relative to control in HCT116 and MCF7 cell lines. Solid bars represent asymmetrically dividing and clear bars represent G0-like cancer cells. Error bars indicate mean±SEM for 3 replicates. FIG. 15D depicts Western blot analysis of short hairpin TTC3 knockdown.

FIGS. 16A-16C demonstrate a mechanism for AKT1low slow proliferators: FAK, mTORC2, and AKT1. FIG. 16A depicts a graphical representation of percentage change in H3K9me2low/MCM2low/HES1 high asymmetrically dividing and G0-like cells relative to control in HCT116 and MCF7 cell lines. Solid bars represent asymmetrically dividing and clear bars represent G0-like cancer cells. Error bars indicate mean±SEM for 3 replicates. FIG. 16B depicts Western blot analysis of short hairpin RICTOR knockdown. FIG. 16C depicts HCT116 and MCF7 cells in M-phase of the cell cycle, FAK IP with anti-FAK and immunoblotted with anti-FAK, anti-mTOR, anti-RICTOR, and anti-RAPTOR antibody. Reciprocally, RICTOR IP with anti-RICTOR and immunoblotted with anti-RICTOR or anti-FAK antibody.

FIGS. 17A-17E demonstrate a mechanism for AKT1low slow proliferators: β1-integrin and FAK. FIGS. 17A-17B depict graphical representation of percentage change in H3K9me2low/MCM2low/HES1high asymmetrically dividing and G0-like cells relative to control in HCT116 and MCF7 cell lines. Solid bars represent asymmetrically dividing and clear bars represent G0-like cancer cells. Error bars indicate mean±SEM for 3 replicates. FIG. 17C depicts bar graphs of percentages of H3K9me2low/MCM2low/HES1high asymmetric mitoses and G0-like cells in MCF7 cells plated on control (random) or aligned type-I collagen fibrils (aligned). FIGS. 17D and 17E depicts Western blots of short hairpin FAK and 31-integrin knockdown in HCT116 cells with nonsilencing shRNA (NS) as control.

### DETAILED DESCRIPTION

Embodiments of the technology described herein relate to the inventor's discovery of a signaling pathway controlling asymmetric cell division and proliferation of cancer cells. Briefly, asymmetric proliferation is induced by the degradation of AKT1 protein. Modulating the rate of degradation of AKT1 protein can thus increase or decrease the rate of asymmetric proliferation and therefore the level of slow proliferator cancer cells within a population of cancer cells. In some embodiments, the degradation of AKT1 can be asymmetric. Methods relating to this modulation are described herein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, the terms “reduced”, “reduction”, “decrease”, or “inhibit” can mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or more or any decrease of at least 10% as compared to a reference level. In some embodiments, the terms can represent a 100% decrease, i.e. a non-detectable level as compared to a reference level. In the context of a marker or symptom, a “decrease” is a statistically significant decrease in such level. The decrease can be, for example, at least 10%, at least 20%, at least 30%, at least 40% or more, and is preferably down to a level accepted as within the range of normal for an individual without such disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. In some embodiments, a cancer cell can be a cell obtained from a tumor.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof The nucleic acid can be either single-stranded or double-stranded. A single-stranded nucleic acid can be one strand nucleic acid of a denatured double- stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer therapy. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

As used herein, the term “AKT1” or “v-akt murine thymoma viral oncogene homolog 1” refers to a serine-threonine protein kinase activated by platelet-derived growth factor. The sequence of AKT1 for a number of species is well known in the art, e.g. human AKT1 (e.g. NCBI Ref Seq: NP_001014431; NCBI Gene ID: 207).

As used herein, the term “mTORC2” refers to mTOR complex 2, a multi-protein complex comprising RICTOR, mTOR, GβL, and MAPKAP1, and which phosphorylates Akt. The sequences of the components of mTORC2 are well known in the art, eg. human mTOR (e.g. NCBI Ref Seq: NP_004949; NCBI Gene ID: 2475), human GβL (e.g. NCBI Ref Seq: NP_001186102; NCBI Gene ID: 64223), and human MAPKAP1 (e.g. NCBI Ref Seq: NP_001006618; NCBI Gene ID: 79109).

As used herein, the term “RICTOR” or “RPTOR independent companion of MTOR, complex 2” refers to a subunit of the mTORC2 complex. The sequence of RICTOR for a number of species is well known in the art, e.g. human RICTOR (e.g. NCBI Ref Seq: NP_689969; NCBI Gene ID: 253260).

As used herein, the term “TTC3” or “tetratricopeptide repeat domain 3” refers to an E3 ligase that controls the ubiquitination of AKT1. The sequence of TTC3 for a number of species is well known in the art, e.g. human TTC3 (e.g. NCBI Ref Seq: NP_001001894; NCBI Gene ID: 7267).

As used herein, the term “focal adhesion kinase” or “FAK” (also known as PTK2 in humans) refers to a tyrosine kinase found at focal adhesions and which is phosphorylated in response to integrin engagement and growth factor perception, thereby regulating cell movement, growth, and survival. The sequence of FAK for a number of species is well known in the art, e.g. human FAK (e.g. NCBI Ref Seq: NP_005598; NCBI Gene ID: 5747).

As described herein, “integrin ” refers to a class of transmembrane receptors that mediate the attachment of a cell to surrounding materials, e.g. extracellular matrix (ECM) or other cells, as well as transduce signals relating to the chemical and mechanical status of the surrounding materials and/or transduce signals from the cell to the surrounding materials. Integrins function as heterodimers, comprising an alpha chain and a beta chain. Mammalian genomes contain eighteen alpha subunits and eight beta subunits. In some embodiments of any of the aspects described herein, an integrin can be a β1-integrin. As described herein, “β1-integrin ” refers to a complete integrin heterodimer comprising a β1 beta chain and any of the eighteen possible alpha chains (e.g. α1-α11, αD, αE, αL, αM, αV, αX or α2B). The sequence of the β1 beta chain (i.e. ITGB1) for a number of species is well known in the art, e.g., human ITGB1 (e.g. NCBI Gene ID: 3688; (mRNA: NCBI Ref Seq: NM_002211) (polypeptide NCBI Ref Seq:NP_002202).

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells and hematopoietic stem cells. In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are totipotent cells derived from the early embryo or blastocyst. Embryonic stem cells can be obtained directly from suitable tissue, including, but not limited to human tissue, or from established embryonic cell lines. In one embodiment, embryonic stem cells are obtained as described by Thomson et al. (U.S. Pat. Nos. 5,843,780 and 6,200,806; Science 282:1145, 1998; Curr. Top. Dev. Biol. 38:133 ff, 1998; Proc. Natl. Acad. Sci. U.S.A. 92:7844, 1995 which are incorporated by reference herein in their entirety).

Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including method for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735. Descriptions of stromal cells, including methods for isolating them, may be found in, among other places, Prockop, Science, 276:71 74, 1997; Theise et al., Hepatology, 31:235 40, 2000; Current Protocols in Cell Biology, Bonifacino et al., eds., John Wiley & Sons, 2000 (including updates through March, 2002); and U.S. Pat. No. 4,963,489.

As used herein, “progenitor cells” refers to cells in an undifferentiated or partially differentiated state and that have the developmental potential to differentiate into at least one more differentiated phenotype, without a specific implied meaning regarding developmental potential (i.e., totipotent, pluripotent, multipotent, etc.) and that does not have the property of self-renewal. Accordingly, the term “progenitor cell” refers to any subset of cells that have the developmental potential, under particular circumstances, to differentiate to a more specialized or differentiated phenotype.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9). Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol.152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

Described herein are methods of modulating asymmetric proliferation in a cancer cell. As used herein, “asymmetric proliferation” refers to a process of cell division in which one daughter cell proliferates at the same rate as the parent cell while the other daughter cell proliferates at a statistically significantly slower rate. These slowly proliferating daughter cells are referred to herein as “slow proliferators.” As used herein, the terms “slow proliferator” or “G0-like cell”, which are used interchangeably herein, refer to a cancer cell which proliferates at a statistically significantly slower rate than the rate observed for at least 70% of cancer cells obtained from the same tumor. In some embodiments, slow proliferators can be cancer cells which have statistically significantly decreased levels of expression of Akt1, H3K9me2, and MCM2 and statistically significantly increased levels of expression of TTC3 and Hes1 as compared to the levels of expression found in at least 70% of cancer cells obtained from the same tumor. In some embodiments, the level of expression of these markers can be the level of polypeptide expression product. In some embodiments, a slow proliferator can revert to a normal, fast-proliferator phenotype, e.g. the slow proliferator phenotype can be reversible.

In one aspect, described herein is a method of modulating the rate of asymmetric proliferation in a cell, the method comprising: contacting the cell with a modulator of AKT1 degradation; wherein an increase in AKT1 degradation increases the rate of asymmetric proliferation in the cell; and wherein a decrease in AKT1 degradation decreases the rate of asymmetric proliferation in the cell. In some embodiments, the rate of asymmetric proliferation in a population of cells can be modulated. As referred to herein, “AKT1 degradation” refers to the ubiquitination and proteasome-mediated degradation of AKT1.

In some embodiments, the cell can be a cancer cell. In some embodiments, the cell can be a stem and/or progenitor cell. In some embodiments, the cell can be a cell engaged in wound repair, e.g. a cell located at a site of a wound and/or defect. In some embodiments, the cell can be a cell undergoing asymmetric division, e.g. cells whose daughter cells comprise slow proliferators. Methods of identifying slow proliferator cells are described elsewhere herein.

A modulator of AKT1 can be an agonist of ATK1 degradation or an inhibitor of AKT1 degradation. Modulators can be agents of any type and/or structure. The term “agent” refers generally to any entity which is normally not present or not present at the levels being administered to a cell, tissue or subject. An agent can be selected from a group comprising: polynucleotides; polypeptides; small molecules; antibodies; or functional fragments thereof. A polynucleotide can be RNA or DNA, and can be single or double stranded, and can be selected from a group comprising: nucleic acids and nucleic acid analogues that encode a polypeptide. A polypeptide can be, but is not limited to, a naturally-occurring polypeptide, a mutated polypeptide or a fragment thereof that retains the function of interest. Further examples of agents include, but are not limited to a nucleic acid (DNA or RNA), small molecule, aptamer, protein, peptide, antibody, polypeptide comprising an epitope-binding fragment of an antibody, antibody fragment, peptide-nucleic acid (PNA), locked nucleic acid (LNA), small organic or inorganic molecules; saccharide; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or mammalian cells or tissues; naturally occurring or synthetic compositions; peptides; aptamers; and antibodies, or fragments thereof. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

In some embodiments, the modulator of AKT1 degradation can be an agonist and/or promoter of AKT1 degradation. An agonist of AKT1 degradation can be any agent that increases the level and/or rate of ATK1 degradation, either through direct or indirect action. As used herein, the term “agonist” refers to an agent which increases the expression and/or activity of the target by at least 10% or more, e.g. by 10% or more, 50% or more, 100% or more, 200% or more, 500% or more, or 1000% or more.

Non-limiting examples of agonists of AKT1 degradation include allosteric inhibitors of AKT1 and clustered homology domain inhibitors of AKT1. In some embodiments, an agonist of AKT1 degradation can be a dual-specific (e.g. in inhibits AKT1 and AKT2) inhibitor or an AKT1-specific inhibitor. Allosteric inhibitors and clustered homology domain inhibitors of AKT1 are known in the art and include, by way of non-limiting example, AKT1/2; ARQ 092; and MK2206. Allosteric inhibitors of AKT1 are also described, e.g. in U.S. Pat. No. 8,183,249; Cherrin et al. Cancer Biol Ther 2010 9:493-503; Calleja et al. PLoS Biol 2009 20: e17; Lindsley et al. Bioorganic & Medicinal Chemistry Letters 2005 15:761-764; Bilodeau et al. Bioorganic and Medicinal Chemistry Letters 2008 18:3178-3182; and Lindsley et al. Current Cancer Drug Targets 2008 8:7-18; which are incorporated by reference herein in their entireties. In some embodiments, an agonist of AKT1 degradation is not a catalytic inhibitor of AKT1.

In some embodiments, contacting a cancer cell with an agonist of ATK1 degradation leads to the production, or the increased production, of slow proliferator cancer cells.

As described herein, AKT1 degradation is negatively regulated by FAK activity. Accordingly, an agonist of ATK1 degradation can include, by way of non-limiting example, an inhibitor of FAK expression and/or activity. Inhibitors of FAK are known in the art, e.g., inhibitory nucleic acids, inhibitor antibody reagents, or small molecules, e.g., PF-562271; NVP-TAE226; PF-573228; Y15; and PND-1186.

As described herein, AKT1 degradation is negatively regulator by β1-integrin activity. Accordingly, an agonist of ATK1 degradation can include, by way of non-limiting example, an inhibitor of β1-integrin expression and/or activity. Non-limiting examples of β1-integrin inhibitors can include inhibitory antibody reagents, e.g., A2B2 and P4C10 antibodies.

In some embodiments, a modulator of AKT1 degradation can be an inhibitor of AKT1 degradation. An inhibitor of AKT1 degradation can be any agent that decreases the level and/or rate of AKT1 degradation, whether by direct or indirect action. As used herein, the term “inhibitor” refers to an agent which reduces the expression and/or activity of the target by at least 10%, e.g. by 10% or more, 20% or more, 30% or more, 50% or more, 75% or more, 90% or more, 95% or more, 98% or more, or 99% or more.

As described herein, AKT1 degradation is positively regulated by mTORC2, RICTOR, and TTC3. Accordingly, inhibiting these proteins and/or expression of these proteins can inhibit AKT1 degradation. Thus, inhibitors of AKT1 degradation include inhibitors of mTORC2 signaling, inhibitors of mTORC2, inhibitors of mTORC2 expression, inhibitors of RICTOR, inhibitors of RICTOR expression, inhibitors of TTC3, and inhibitors of TTC3 expression. AKT1 degradation is negatively regulated by β-integrin activity. Accordingly, activating or increasing β-integrin expression or activity can inhibit AKT1 degradation. Thus, inhibitors of AKT1 degradation include activators of β-integrin activity and activators of β-integrin expression.

Further, irregular concentrations of collagen in the extracellular environment can create polar activation of β-integrin by the collagen, which can increase AKT1 degradation. Accordingly, providing a substrate or growth medium for a cell such that the individual cell is exposed to a homogeneous concentration of collagen can inhibit AKT1 degradation. In some embodiments, the substrate or growth medium with a homogenous concentration of collagen can comprise a substrate or growth medium with a structural collage matrix having a fibrillar pattern.

In some embodiments, inhibitors of AKT1 degradation can include an inhibitor of ATK1 expression, e.g. an inhibitory nucleic acid.

In some embodiments, inhibitors of AKT1 degradation can include an agonist of β1-integrin, e.g. a nucleic acid encoding β1-integrin or an activating antibody reagent. Such reagents are known in the art, e.g., the TS2/16 and 12G10 monoclonal antibodies.

Inhibitors of the expression of a given gene can be an inhibitory nucleic acid. For example, gene silencing or RNAi can be used. In certain embodiments, contacting a cell with the inhibitor results in a decrease in the target mRNA level in a cell of at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one embodiment, the mRNA levels are decreased by at least about 70%, about 80%, about 90%, about 95%, about 99%, about 100%. In certain embodiments, the inhibitor can comprise an expression vector or viral vector comprising the RNAi molecule.

As used herein, the term “RNAi” refers to any type of interfering RNA, including but are not limited to RNAi, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” and “RNA interfering” with respect to an agent of the technology described herein, are used interchangeably herein.

As used herein a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

RNAi may be delivered with the help of nanoparticles as described for example in Schiffelers and Storm, Expert Opin Drug Deliv. 2006 May;3(3):445-54 or liposomes (e.g. Hughes et al., Methods Mol Biol. 2010;605:445-59).

Inhibitors of mTORC2 are known in the art and include, by way of non-limiting example, TORIN1, AZD8055, INK128, and Palomid-529. Further examples of mTORC2 inhibitors include OSI-027; MK8669; TOP216; TORISEL; CERTICAN; ABI-009; KU-0063794; AZD2014; NVP-BGT226; PF-04691502; PP242; XL765; EXEL-2044; EXEL-3885; EXEL-4431; EXEL-7518 and those described, e.g. in US Patent Publication 2012/0165334; 2011/0224223; 2012/0114739; 2010/0184760; 2012/0178715; Bhagwat and Crew. Curr Opin Investig Drugs 2010 11:638-645; which are incorporated by reference herein in their entireties. In some embodiments, an inhibitor of mTORC2 can be an inhibitor of mTORC1 and mTORC2. In some embodiments, an inhibitor of mTORC2 can be specific for inhibition of mTORC2.

Inhibitors of RICTOR are known in the art and include, by way of non-limiting example, NVP-BEZ235.

Inhibitors of TTC3 are known in the art and include, by way of non-limiting example, MG-132 and bortezomib.

Inhibitors of FAK are known in the art and include, by way of non-limiting example, PF-562271 and NVP-TAE226.

Inhibitors of β-integrin activity are known in the art and include, by way of non-limiting example, the monoclonal antibodies A2B2 and P4C10.

Therapies which target fast proliferator cells can be ineffective in decreasing populations of slow proliferators (see, e.g. Dey-Guha et al. PNAS 2011108:12845-12850; which is incorporated by reference herein in its entirety). Accordingly, in one aspect, described herein is a method of treating cancer in a subject in need thereof, the method comprising: administering an inhibitor of AKT1 degradation to the subject (i.e. decreasing the number of slow proliferators in the subject). In some embodiments, the method can further comprise administering a cancer therapy that targets fast proliferator cancer cells. In some embodiments, the inhibitor of AKT1 degradation can be administered before the administration of a cancer therapy that targets fast proliferator cancer cells. In some embodiments, the inhibitor of AKT1 degradation can be administered at least 1 day before the administration of a cancer therapy that targets fast proliferator cancer cells, e.g. 1 day, 2 days, 3 days, 4 days, 5 days, 6 days, 7 days or further before. In some embodiments, the inhibitor of AKT1 degradation can be administered at least 3 days before administration of a cancer therapy that targets fast proliferator cancer cells. Cancer therapies that target fast proliferator cells are well known in the art and include, by way of non-limiting example, therapies that degrade or disrupt nucleic acids, e.g. doxorubicin, alkylating agents, nitrogen mustard alkylating agents, agents that intercalate DNA; cyclophosphamide, or therapies that inhibit cell division, e.g. mitotic inhibitors, paclitaxel.

In some embodiments, an inhibitor of AKT1 degradation can be administered to reduce and/or reverse the growth of a cancer. In some embodiments, an inhibitor of AKT1 degradation can be administered to reduce the rate of the growth of a cancer. In some embodiments, an inhibitor of AKT1 degradation can be administered to prevent the growth of a cancer. In some embodiments, an inhibitor of AKT1 degradation can be administered to prevent relapse and/or development of a cancer.

Further examples of agents that can be fast proliferator targeting agents include, but are not limited to chemotherapeutic agents, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., Herceptin®), anti-CD20 antibodies, an epidermal growth factor receptor (EGFR) antagonist (e.g., a tyrosine kinase inhibitor), HER1/EGFR inhibitor (e.g., erlotinib (Tarceva®)), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention. The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re186, Re¹⁸⁸, Sm¹⁵³, Bi²¹², P³² and radioactive isotopes of Lu), chemotherapeutic agents, and toxins, such as small molecule toxins or enzymatically active toxins of bacterial, fungal, plant or animal origin, including fragments and/or variants thereof As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2.sup.nd ed., 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In some embodiments, the modulators of AKT1 degradation described herein can be used in conjunction with additional chemotherapeutic agents. By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

In some embodiments, the subject can be one who has been identified has having slow proliferator cells. In some embodiments, the subject has been determined to have a subpopulation of cancer cells expressing increased levels of one or more genes selected from the group consisting of: Hes1 and TTC3; and/or decreased levels of one or more genes selected from the group consisting of: AKT1; H3K9me2; and MCM2; wherein an increased level is a level statistically significantly higher than the level of expression found in at least 70% of cancer cells obtained from the same tumor and a decreased level is a level statistically significantly lower than the level of expression found in at least 70% of cancer cells obtained from the same tumor. In some embodiments, the subject can have been determined to have cancer cells expressing increased levels of TTC3; and, optionally, increased levels of Hes1 and/or decreased levels of one or more genes selected from the group consisting of: AKT1; H3K9me2; and MCM2. In some embodiments, the subject can have been determined to have cancer cells expressing increased levels of Hesland TTC3 and decreased levels AKT1; H3K9me2; and MCM2. In some embodiments, an increased level can be at least 2× higher than the level of expression found in at least 70% of cancer cells obtained from the same tumor, e.g. at least 2×, at least 3×, at least 4×, at least 5×, at least 10×, or higher. In some embodiments, an increased level can be at least 50% or less than the level of expression found in at least 70% of cancer cells obtained from the same tumor, e.g. 50% or less, 40% or less, 30% or less, 20% or less, 10% or less, or less.

The expression level of a gene can be the level of mRNA or polypeptide expression product. In some embodiments, the level of expression can be determined, e.g. by in situ hybridization and/or immunochemistry of biopsies or tissue samples. In some embodiments, the expression level of an mRNA expression product can be determined, e.g. by RT-PCR, quantitative RT-PCR, RNA-seq, Northern blot, or microarray based expression analysis. In some embodiments, the level of expression of a gene can be the level of polypeptide expression product. Methods for measuring polypeptide expression products are known in the art and include, by way of non-limiting example ELISA (enzyme linked immunosorbent assay), western blot, immunoprecipitation, immunohistochemistry, and immunofluorescence using detection reagents such as an antibody or protein binding agent. In some embodiments, the expression level can be determined by immunochemistry. Methods of detecting the expression level of slow proliferator markers have been described, e.g. in Dey-Guha et al. PNAS 2011108:12845-12850: which is incorporated by reference herein in its entirety and in the Examples herein.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience, e.g. a change in color, upon encountering the targeted molecules or upon treatment with a chemical agent. In some instances, signal amplification can be integrated into the particular protocol, wherein a secondary antibody, that includes the marker signal or marker activity (e.g. an enzyme activity), follows the application of a primary target-specific antibody.

In one aspect, described herein is a kit for determining if a subject has slow proliferator cells. In some embodiments, the kit can comprise a detection agent specific for an expression product of TTC3. In some embodiments, the kit can comprise a detection agent specific for an expression product of at least one of the genes selected from the group consisting of: TTC3; Hes1; AKT1; H3K9me2; MCM2; MK167; CDC6; GMNN; AURKA; PLK1; H3S10ph; H3K4me2; H3K9me2; H3K27me3; H4K12ac; and H4K16ac. A detection agent can be any agent which can specifically detect the presence of the target (e.g. bind specifically to the target) according to an assay described herein, e.g. a detection reagent can be a nucleic acid probe or primer specific for the target or an agent which specifically binds to a target polypeptide. In some embodiments, the detection reagent can comprise a detectable signal or be capable of generating a detectable signal. In some embodiments, the detection agent can be an antibody reagent. In some embodiments, the detection agent can be a monoclonal antibody and/or comprise CDRs of a monoclonal antibody. Non-limiting examples of antibody reagents specific for the described slow proliferator markers are described in the Examples herein. In some embodiments, the kit can further comprise reagents necessary for performing the assay, e.g. buffers and/or reagents for generating and/or detecting a detectable signal. In some embodiments, the kit can further comprise instructions.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeable herein are used herein to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those of skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity.

The term “label” refers to a composition capable of producing a detectable signal indicative of the presence of an antibody reagent (e.g. a bound antibody reagent). Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

In some cases, the rate of growth of a cancer (e.g. a tumor) can be reduced by increasing the percentage of the cells which are slow proliferators. Accordingly, in one aspect, described herein is a method of treating cancer in a subject in need thereof, the method comprising: administering an agonist of AKT1 degradation to the subject. Administration of an agonist of ATK1 degradation can increase the number of slow proliferators present in a tumor, causing the overall growth rate of the tumor to decrease. In some embodiments, the subject can be a subject selected from the group consisting of: a subject with early stage cancer; a subject who is in remission or is likely to be in remission; a subject at risk of developing cancer and/or a subject at risk of having a cancer and/or tumor grow to the extent that it is clinically dangerous.

Described herein are treatments for cancer. The cancer to be treated can be any type of cancer in any location. In some embodiments, the cancer can be breast cancer, lung cancer, prostate cancer, colorectal cancer, lung cancer, and/or melanoma. In some embodiments, the cancer can comprise a metastasis

In one aspect, described herein is a method of producing slow proliferator cancer cells, the method comprising: (i) contacting a cancer cell with an agonist of AKT1 degradation; (ii) maintaining the cancer cells treated in step (i). The cells can be maintained in vivo or in vitro. Conditions suitable for maintaining cells in culture are well known in the art and can vary depending on the precise identity of the cells. In some embodiments, slow proliferators can be maintained under the same cell culture conditions as the cancer cells from which they originated. Examples of suitable cell culture conditions are described in the Examples herein. In some embodiments, the method can further comprise the step of enriching the cells of step (ii) for slow proliferators by selecting for cells having increased levels of expression of TTC3 and optionally, increased levels of expression of Hes1 or decreased levels of expression of AKT1; H3K9me2; MCM2; MK167; CDC6; GMNN; AURKA; PLK1; H3S10ph; H3K4me2; H3K9me2; H3K27me3; H4K12ac; or H4K16ac. In some embodiments, the method can further comprise the step of enriching the cells of step (ii) for slow proliferators by selecting for cells having increased levels of expression of TTC3 and Hes1 and decreased levels of expression of AKT1; H3K9me2; MCM2; MK167; CDC6; GMNN; AURKA; PLK1; H3S10ph; H3K4me2; H3K9me2; H3K27me3; H4K12ac; and H4K16ac. Enriching can encompass selecting for slow proliferators (e.g. treating with an agent specific for fast proliferators) or sorting slow proliferators from other cells, e.g. by FACS sorting.

In one aspect, described herein is a method of screening for an anti-slow proliferator agent, the method comprising: (i) contacting a cancer cell with an agonist of AKT1 degradation; (ii) contacting the cell obtained from step (i) with a test agent; (iii) determining the anti-tumor effect of the test agent; (iv) identifying a test agent as an anti-slow proliferator agent when a statistically significant anti-tumor effect is observed. As described herein, an “anti-slow proliferator agent” is any agent which can either 1) cause slow proliferators to convert to a fast proliferator phenotype (e.g. proliferate at a fast proliferator rate) or 2) selectively kill slow proliferators. As described herein, an anti-tumor effect can comprise a reduction in the growth of a tumor, a reduction in signs or symptoms of cancer, a reduction in mortality, cytotoxic activity, cytotoxicity specific for slow proliferators, reduction in relapse after remission, a reduction in invasiveness, and/or a reduction of metastasis. One of ordinary skill in the art readily appreciates how to measure such phenotypes, e.g. by cell viability assays, or by measuring the size of tumors over time.

As described herein, the term “test agent” refers to a compound or agent and/or compositions thereof that are to be screened to determine whether they possess anti-tumor and/or anti-slow proliferator activity, as identified herein. In the context of the screening methods described herein, a “test agent” can be a nucleic acid (DNA or RNA), small molecule, aptamer, protein, peptide, antibody, polypeptide comprising an epitope-binding fragment of an antibody, antibody fragment, peptide-nucleic acid (PNA), locked nucleic acid (LNA), small organic or inorganic molecules; saccharide; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or mammalian cells or tissues; naturally occurring or synthetic compositions; peptides; aptamers; and antibodies, or fragments thereof.

The methods of screening described herein can be performed in vitro or in vivo. In some embodiments, the cancer cell contacted with the agonist of AKT1 degradation is located and/or maintained in vivo, e.g. in an animal model of cancer. In some embodiments, the cancer cell contacted with the agonist of AKT1 degradation is located and/or maintained in vitro, e.g. in cell culture. In some embodiments, the method can further comprise selecting for slow proliferator cells after step (i), e.g. sorting cells by FACS using the slow proliferator markers described herein (e.g. TTC3).

In some embodiments, contacting a population of cancer cells with a agonist of AKT1 degradation can increase the number and/or proportion of slow proliferators in the population by a statistically significant amount. In some embodiments, contacting a population of cancer cells with a agonist of AKT1 degradation can increase the number and/or proportion of slow proliferators in the population by at least 2×, e.g. 2× or more, 3× or more, 4× or more, 5× or more, 6× or more, 7× or more, 8× or more, 9× or more, 10× or more, 20× or more, 50× or more, or 100× or more.

Further, the methods of screening described herein can also be adapted to screen for agents which cause slow proliferators to retain a slow proliferator phenotype (i.e. cause a lower rate of reversion to a fast proliferator phenotype as compared to untreated cells) or agents which cause slow proliferators to enter a dormant or quiescent state. One of skill in the art readily appreciates how to screen for such phenotypes, e.g. by measuring proliferation rates and/or metabolic rates.

In one aspect, described herein is a method comprising; (i) obtaining a tumor biopsy from a subject; (ii) determining the expression level of TTC3 in cells obtained from the subject; (iii) identifying the presence of slow proliferators in the tumor when cells with increased levels of expression of TTC3 are detected. In some embodiments, the expression level of TTC3 and optionally Hes1, AKT1; H3K9me2; MCM2; MK167; CDC6; GMNN; AURKA; PLK1; H3S10ph; H3K4me2; H3K9me2; H3K27me3; H4K12ac; or H4K16ac can be determined; wherein the presence of slow proliferators in the tumor is indicated when cells with increased levels of expression of TTC3 and optionally increased levels of expression of Hes1 or decreased levels of expression of AKT1; H3K9me2; MCM2; MK167; CDC6; GMNN; AURKA; PLK1; H3S10ph; H3K4me2; H3K9me2; H3K27me3; H4K12ac; or H4K16ac are detected. In some embodiments, the method can further comprise administering an inhibitor of AKT1 degradation to the subject. In some embodiments, the method can further comprise treating the cancer with an inhibitor of AKT1 degradation according to any of the embodiments described herein. The determination of the expression level of the expression products foregoing genes (excepting TTC3) can be performed as described in, e.g. Dey-Guha et al. PNAS 2011108:12845-12850; which is incorporated by reference herein in its entirety.

In one aspect, described herein is a method of screening for a biomarker of anti-slow proliferator cells, the method comprising: (i) contacting a cancer cell with an agonist of AKT1 degradation; (ii) measuring the expression of one or more genes in the cell of (i) and comparing that to the level of expression to a reference level (e.g. the level in the cell prior to step (i) or to a cell not treated according to step (i)), wherein a gene having expression after step (i) which varies by a statistically significant amount is identified as a biomarker of slow proliferator status. One of ordinary skill in the art readily appreciates how to measure such gene expression levels, e.g. by microarray.

Administration

In some embodiments, the methods described herein relate to treating a subject having or diagnosed as having cancer with a modulator of AKT1 degradation. Subjects having cancer can be identified by a physician using current methods of diagnosing cancer. Symptoms and/or complications of cancer which characterize these conditions and aid in diagnosis are well known in the art and include but are not limited to, growth of a tumor, impaired function of the organ or tissue harboring cancer cells, etc. Tests that may aid in a diagnosis of, e.g. cancer include, but are not limited to, tissue biopsies and histological examination. A family history of cancer or exposure to risk factors for cancer (e.g. smoking or radiation) can also aid in determining if a subject is likely to have cancer or in making a diagnosis of cancer.

The compositions and methods described herein can be administered to a subject having or diagnosed as having cancer. In some embodiments, the methods described herein comprise administering an effective amount of compositions described herein, e.g. modulators of ATK1 degradation to a subject in order to alleviate a symptom of a cancer. As used herein, “alleviating a symptom of a cancer” is ameliorating any condition or symptom associated with the cancer. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a modulator of ATK1 degradation needed to alleviate at least one or more symptom of the disease or disorder, and relates to a sufficient amount of pharmacological composition to provide the desired effect. The term “therapeutically effective amount” therefore refers to an amount of a modulator of ATK1 degradation that is sufficient to effect a particular anti-tumor effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a modulator of AKT1 degradation, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for tumor growth, among others. The dosage can be determined by a physician and adjusted, as necessary, to suit observed effects of the treatment.

In some embodiments, the technology described herein relates to a pharmaceutical composition comprising a modulator of ATK1 degradation as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a modulator of ATK1 degradation as described herein.

In some embodiments, the pharmaceutical composition comprising a modulator of ATK1 degradation as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of a modulator of ATK1 degradation as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a modulator of ATK1 degradation as disclosed herein can also be incorporated into the parenteral dosage forms of the disclosure, including conventional and controlled-release parenteral dosage forms.

Pharmaceutical compositions comprising a modulator of ATK1 degradation can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Phila. Pa. (2005); which is incorporated by reference herein in its entirety.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the agent can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000); which is incorporated by reference herein in its entirety.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE™. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb™); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In addition, the methods of treatment can further include the use of radiation or radiation therapy. Further, the methods of treatment can further include the use of surgical treatments.

In certain embodiments, an effective dose of a composition comprising a modulator of ATK1 degradation as described herein can be administered to a patient once. In certain embodiments, an effective dose of a composition comprising a modulator of ATK1 degradation can be administered to a patient repeatedly. For systemic administration, subjects can be administered a therapeutic amount of a composition comprising a modulator of ATK1 degradation such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. A composition comprising a modulator of ATK1 degradation can be administered over a period of time, such as over a 5 minute, 10 minute, 15 minute, 20 minute, or 25 minute period. The administration can be repeated, for example, on a regular basis, such as hourly for 3 hours, 6 hours, 12 hours or longer or such as biweekly (i.e., every two weeks) for one month, two months, three months, four months or longer.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. cancer by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active agent. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more.

The dosage ranges for the administration of a modulator of ATK1 degradation according to the methods described herein depend upon, for example, the form of a modulator of ATK1 degradation, its potency, and the extent to which symptoms, markers, or indicators of a condition described herein are desired to be reduced, for example the percentage reduction desired for cancer growth or the extent to which, for example, tumor size are desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a modulator of ATK1 degradation in, e.g. the treatment of a condition described herein, or to induce a response as described herein (e.g. reduction in tumor growth) can be determined by the skilled clinician. However, a treatment is considered “effective treatment,” as the term is used herein, if any one or all of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. reduction in tumor growth rate). It is well within the ability of one skilled in the art to monitor efficacy of administration and/or treatment by measuring any one of such parameters, or any combination of parameters. Efficacy can be assessed in animal models of a condition described herein, for example treatment of cancer. When using an experimental animal model, efficacy of treatment is evidenced when a statistically significant change in a marker is observed, e.g. tumor size.

In vitro and animal model assays are provided herein which allow the assessment of a given dose of a modulator of ATK1 degradation. By way of non-limiting example, the effects of a dose of a modulator of ATK1 degradation can be assessed by monitoring the growth of a xenograft of cancer cells in mice as described herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of modulating the rate of asymmetric proliferation in a cell, the method comprising:
   contacting the cell with a modulator of AKT1 degradation;
   wherein an increase in AKT1 degradation increases the rate of asymmetric proliferation in the cell; and
   wherein a decrease in AKT1 degradation decreases the rate of asymmetric proliferation in the cell.
2. The method of paragraph 1, wherein the cell is selected from the group consisting of:
   a cancer cell; a stem cell; a progenitor cell; and a cell engaged in wound repair.
3. The method of any of paragraphs 1-2, wherein the modulator of AKT1 degradation is an agonist of AKT1 degradation selected from the group consisting of:
   an allosteric inhibitor of AKT1; AKT1/2; MK2206; an inhibitor of β-integrin expression; an inhibitor of β-integrin activity; A2B2: P4C10; an inhibitor of focal adhesion kinase (FAK) expression; an inhibitor of FAK activity; PF-562271; and NVP-TAE226.
4. The method of paragraph 3, whereby slow proliferator cancer cells are produced.
5. The method of any of paragraphs 1-2, wherein the modulator of AKT1 degradation is an inhibitor of AKT1 degradation selected from the group consisting of:
   inhibitors of mTOR complex 2 (mTORC2) signaling; inhibitors of mTORC2; TORIN1; AZD8055; INK128; Palomid-529; inhibitors of mTORC2 expression;
   inhibitors of RPTOR independent companion of MTOR, complex 2 (RICTOR);
   inhibitors of RICTOR expression; an inhibitor of tetratricopeptide repeat domain 3 (TTC3); MG-132; bortezomib; activators of β-integrin activity; activators of (β-integrin expression.
6. The method of any of paragraphs 1-2, wherein the modulator of AKT1 degradation is a substrate or growth medium which provides a homogeneous concentration of collagen to an individual cell;
   wherein AKT1 degradation is inhibited by the symmetric activation of β-integrin by the homogeneous concentrations of collagen.
7. A method of treating cancer in a subject in need thereof, the method comprising:
   administering an inhibitor of AKT1 degradation to the subject.
8. The method of paragraph 7, wherein the inhibitor of AKT1 degradation is selected from the group consisting of:
   inhibitors of mTOR complex 2 (mTORC2) signaling; inhibitors of mTORC2; TORIN1; AZD8055; INK128; Palomid-529; inhibitors of mTORC2 expression;
   inhibitors of RPTOR independent companion of MTOR, complex 2 (RICTOR);
   inhibitors of RICTOR expression; an inhibitor of tetratricopeptide repeat domain 3 (TTC3); MG-132; bortezomib; activators of β-integrin activity; activators of β-integrin expression.
9. The method of any of paragraphs 7-8, wherein the cancer is selected from the group consisting of:
   melanoma; lung cancer; colorectal cancer; and breast cancer.
10. The method of any of paragraphs 7-9, wherein the method further comprises administering a cancer therapy that targets fast proliferator cancer cells.
11. The method of paragraph 10, wherein the inhibitor of AKT1 degradation is administered before the administration of a cancer therapy that targets fast proliferator cancer cells.
12. The method of paragraph 11, wherein the inhibitor of AKT1 degradation is administered at least 1 day before the administration of a cancer therapy that targets fast proliferator cancer cells.
13. The method of paragraph 12, wherein the inhibitor of AKT1 degradation is administered at least 3 days before the administration of a cancer therapy that targets fast proliferator cancer cells.
14. The method of any of paragraphs 7-13, wherein the subject has been determined to have a subpopulation of cancer cells expressing increased levels of one or more genes selected from the group consisting of:
   Hes1 and TTC3;
   or decreased levels of one or more genes selected from the group consisting of: AKT1; H3K9me2; MCM2; MK167; CDC6; GMNN; AURKA; PLK1; H3S10ph; H3K4me2; H3K9me2; H3K27me3; H4K12ac; and H4K16ac.
   wherein an increased level is a level statistically significantly higher than the level of expression found in at least 70% of cancer cells obtained from the same tumor and a decreased level is a level statistically significantly lower than the level of expression found in at least 70% of cancer cells obtained from the same tumor.
15. The method of paragraph 14, wherein the subject has been determined to have cancer cells expressing increased levels of TTC3; and optionally,
   increased levels of Hes1;
   or decreased levels of one or more genes selected from the group consisting of: AKT1; H3K9me2; MCM2; MK167; CDC6; GMNN; AURKA; PLK1; H3S10ph; H3K4me2; H3K9me2; H3K27me3; H4K12ac; and H4K16ac.
16. The method of paragraph 15, wherein the subject has been determined to have cancer cells expressing increased levels of Hes1 and TTC3 and decreased levels AKT1; H3K9me2; and MCM2.
17. The method of any of paragraphs 14-16, wherein the expression level of the one or more genes is the level of polypeptide expression product.
18. The method of any of paragraphs 14-17, wherein the expression level is determined by immunochemistry.
19. A method of treating cancer in a subject in need thereof, the method comprising:
   administering an agonist of AKT1 degradation to the subject.
20. The method of paragraph 19, wherein agonist of AKT1 degradation is selected from the group consisting of:
   an allosteric inhibitor of AKT1; AKT1/2; MK2206; an inhibitor of β-integrin expression; an inhibitor of β-integrin activity; A2B2: P4C10; an inhibitor of focal adhesion kinase (FAK) expression; an inhibitor of FAK activity; PF-562271; and NVP-TAE226.
21. The method of paragraph 20, wherein the subject is a subject selected from the group consisting of:
   a subject with early stage cancer; a subject who is in remission or is likely to be in remission; and a subject at risk of developing cancer.
22. A method of screening for an anti-slow proliferator agent, the method comprising:
   (i) contacting a cancer cell with an agonist of AKT1 degradation;
   (ii) contacting the cell obtained from step (i) with a test agent;
   (iii) determining the anti-tumor effect of the test agent;
   (iv) identifying a test agent as an anti-slow proliferator agent when a statistically significant anti-tumor effect is observed.
23. The method of paragraph 22, wherein the cancer cell is maintained in vitro.
24. The method of paragraph 22, wherein the cancer cell is maintained in vivo.
25. A method comprising;
   (i) obtaining a tumor biopsy from a subject;
   (ii) determining the expression level of TTC3 in cells obtained from the subject;
   (iii) identifying the presence of slow proliferators in the tumor when cells with increased levels of expression of TTC3 are detected.
26. The method of paragraph 25, wherein the expression level of TTC3 and optionally Hes1, AKT1; H3K9me2; MCM2; MK167; CDC6; GMNN; AURKA; PLK1; H3S10ph; H3K4me2; H3K9me2; H3K27me3; H4K12ac; or H4K16ac are determined; and
   wherein the presence of slow proliferators in the tumor is indicated when cells with increased levels of expression of TTC3 and optionally,
   increased levels of expression of Hes1 or decreased levels of expression of AKT1; H3K9me2; MCM2; MK167; CDC6; GMNN; AURKA; PLK1; H3S10ph; H3K4me2; H3K9me2; H3K27me3; H4K12ac; or H4K16ac are detected.
27. The method of any of paragraphs 25-26, wherein the method further comprises administering an inhibitor of AKT1 degradation to the subject to a subject identified as having cancer cells with increased levels of expression of TTC3.
28. A method of producing slow proliferator cancer cells, the method comprising:
   (i) contacting a cancer cell with an agonist of AKT1 degradation;
   (ii) maintaining the cancer cells treated in step (i).
29. The method of paragraph 28, wherein the method further comprises the step of enriching the cells of step (ii) for slow proliferators by selecting for cells having increased levels of expression of TTC3 and optionally, increased levels of expression of Hes1 or decreased levels of expression of AKT1; H3K9me2; MCM2; MK167; CDC6; GMNN; AURKA; PLK1; H3S10ph; H3K4me2; H3K9me2; H3K27me3; H4K12ac; or H4K16ac.
30. The method of paragraph 29, wherein the method further comprises the step of enriching the cells of step (ii) for slow proliferators by selecting for cells having increased levels of expression of TTC3 and Hes1 and decreased levels of expression of AKT1; H3K9me2; MCM2; MK167; CDC6; GMNN; AURKA; PLK1; H3S10ph; H3K4me2; H3K9me2; H3K27me3; H4K12ac; and H4K16ac.
31. A kit for performing the method of any of paragraphs 25-30.
32. The kit of paragraph 31, wherein the kit comprises a detection agent specific for an expression product of TTC3.
33. The kit of any of paragraphs 31-32, wherein the kit comprises a detection agent specific for an expression product of at least one of the genes selected from the group consisting of: TTC3; Hes1 ; AKT1; H3K9me2; MCM2; MK167; CDC6; GMNN; AURKA; PLK1; H3S10ph; H3K4me2; H3K9me2; H3K27me3; H4K12ac; and H4K16ac.
34. The kit of any of paragraphs 31-33, wherein the detection agent is an antibody reagent.
35. The use of an inhibitor of AKT1 degradation to treat cancer, the use comprising administering an inhibitor of AKT1 degradation to a subject in need of treatment for cancer.
36. The use of paragraph 35, wherein the an inhibitor of AKT1 degradation is selected from the group consisting of:
   inhibitors of mTOR complex 2 (mTORC2) signaling; inhibitors of mTORC2; TORIN1; AZD8055; INK128; Palomid-529; inhibitors of mTORC2 expression;
   inhibitors of RPTOR independent companion of MTOR, complex 2 (RICTOR);
   inhibitors of RICTOR expression; an inhibitor of tetratricopeptide repeat domain 3 (TTC3); MG-132; bortezomib; activators of β-integrin activity; activators of β-integrin expression.
37. The use of any of paragraphs 35-36, wherein the cancer is selected from the group consisting of:
   melanoma; lung cancer; colorectal cancer; and breast cancer.
38. The use of any of paragraphs 35-37, wherein the subject is further administered a cancer therapy that targets fast proliferator cancer cells.
39. The use of paragraph 38, wherein the inhibitor of AKT1 degradation is administered before the administration of a cancer therapy that targets fast proliferator cancer cells.
40. The use of paragraph 39, wherein the inhibitor of AKT1 degradation is administered at least 1 day before the administration of a cancer therapy that targets fast proliferator cancer cells.
41. The use of paragraph 39, wherein the inhibitor of AKT1 degradation is administered at least 3 days before the administration of a cancer therapy that targets fast proliferator cancer cells.
42. The use of any of paragraphs 35-41, wherein the subject has been determined to have a subpopulation of cancer cells expressing increased levels of one or more genes selected from the group consisting of:
   Hes1 and TTC3;
   or decreased levels of one or more genes selected from the group consisting of: AKT1; H3K9me2; MCM2; MK167; CDC6; GMNN; AURKA; PLK1; H3S10ph; H3K4me2; H3K9me2; H3K27me3; H4K12ac; and H4K16ac.
   wherein an increased level is a level statistically significantly higher than the level of expression found in at least 70% of cancer cells obtained from the same tumor and a decreased level is a level statistically significantly lower than the level of expression found in at least 70% of cancer cells obtained from the same tumor.
43. The use of paragraph 42, wherein the subject has been determined to have cancer cells expressing increased levels of TTC3; and optionally,
   increased levels of Hes1;
   or decreased levels of one or more genes selected from the group consisting of: AKT1; H3K9me2; MCM2; MK167; CDC6; GMNN; AURKA; PLK1; H3S10ph; H3K4me2; H3K9me2; H3K27me3; H4K12ac; and H4K16ac.
44. The use of paragraph 43, wherein the subject has been determined to have cancer cells expressing increased levels of Hes1 and TTC3 and decreased levels AKT1; H3K9me2; and MCM2.
45. The use of any of paragraphs 42-44, wherein the expression level of the one or more genes is the level of polypeptide expression product.
46. The use of any of paragraphs 42-45, wherein the expression level is determined by immunochemistry.
47. The use of an agonist of AKT1 degradation to treat cancer, the use comprising administering an agonist of AKT1 degradation to a subject in need of treatment for cancer.
48. The use of paragraph 47, wherein the agonist of AKT1 degradation is selected from the group consisting of:
   an allosteric inhibitor of AKT1; AKT1/2; MK2206; an inhibitor of β-integrin expression; an inhibitor of β-integrin activity; A2B2: P4C10; an inhibitor of focal adhesion kinase (FAK) expression; an inhibitor of FAK activity; PF-562271; and NVP-TAE226.
49. The use of paragraph 48, wherein the subject is a subject selected from the group consisting of:
   a subject with early stage cancer; a subject who is in remission or is likely to be in remission; and a subject at risk of developing cancer.

Some embodiments of the technology described herein can be defined according to any of the following numbered paragraphs:
1. A method of modulating the rate of asymmetric proliferation in a cancer cell, the method comprising:
   contacting the cancer cell with a modulator of AKT1 degradation;
   wherein an increase in AKT1 degradation increases the rate of asymmetric proliferation in the cancer cell; and
   wherein a decrease in AKT1 degradation decreases the rate of asymmetric proliferation in the cancer cell.
2. The method of paragraph 1, wherein the modulator of AKT1 degradation is an agonist of AKT1 degradation selected from the group consisting of:
   an allosteric inhibitor of AKT1; an allosteric inhibitor of AKT1/2; MK2206; an inhibitor of FAK; an inhibitor of β1-integrin; PF-562271; and NVP-TAE226.
3. The method of paragraph 2, whereby slow proliferator cancer cells are produced.
4. The method of paragraph 1, wherein the modulator of AKT1 degradation is an inhibitor of AKT1 degradation selected from the group consisting of:
   inhibitors of mTORC2 signaling; inhibitors of mTORC2; TORIN1; AZD8055; INK128; Palomid-529; inhibitors of mTORC2 expression; inhibitors of RICTOR;
   inhibitors of RICTOR expression; an inhibitor of TTC3; MG-132; bortezomib; an
   inhibitor of ATK1 expression; an agonist of β1-integrin ; and a cell medium comprising a fibrillar pattern of collagen.
5. A method of treating cancer in a subject in need thereof, the method comprising:

administering an inhibitor of AKT1 degradation to the subject.
6. The method of paragraph 5, wherein the method further comprises administering a cancer therapy that targets fast proliferator cancer cells.
7. The method of paragraph 6, wherein the inhibitor of AKT1 degradation is administered before the administration of a cancer therapy that targets fast proliferator cancer cells.
8. The method of paragraph 7, wherein the inhibitor of AKT1 degradation is administered at least 1 day before the administration of a cancer therapy that targets fast proliferator cancer cells.
9. The method of paragraph 7, wherein the inhibitor of AKT1 degradation is administered at least 3 days before the administration of a cancer therapy that targets fast proliferator cancer cells.
10. The method of any of paragraphs 5-9, wherein the subject has been determined to have a subpopulation of cancer cells expressing increased levels of one or more genes selected from the group consisting of:
   Hes1 and TTC3;
   or decreased levels of one or more genes selected from the group consisting of: AKT1; H3K9me2; MCM2; MK167; CDC6; GMNN; AURKA; PLK1; H3S10ph; H3K4me2; H3K9me2; H3K27me3; H4K12ac; and H4K16ac.
   wherein an increased level is a level statistically significantly higher than the level of expression found in at least 70% of cancer cells obtained from the same tumor and a
   decreased level is a level statistically significantly lower than the level of expression found in at least 70% of cancer cells obtained from the same tumor.
11. The method of paragraph 10, wherein the subject has been determined to have cancer cells expressing increased levels of TTC3; and optionally,
   increased levels of Hes1 ;
   or decreased levels of one or more genes selected from the group consisting of: AKT1; H3K9me2; MCM2; MK167; CDC6; GMNN; AURKA; PLK1; H3S10ph; H3K4me2; H3K9me2; H3K27me3; H4K12ac; and H4K16ac.
12. The method of paragraph 10, wherein the subject has been determined to have cancer cells expressing increased levels of Hes1 and TTC3 and decreased levels AKT1; H3K9me2; and MCM2.
13. The method of any of paragraphs 10-12, wherein the expression level of the one or more genes is the level of polypeptide expression product.
14. The method of any of paragraphs 10-13, wherein the expression level is determined by immunochemistry.
15. A method of treating cancer in a subject in need thereof, the method comprising:
   administering an agonist of AKT1 degradation to the subject.
16. The method of paragraph 15, wherein the subject is a subject selected from the group consisting of:
   a subject with early stage cancer; a subject who is in remission or is likely to be in remission; and a subject at risk of developing cancer.
17. A method of screening for an anti-slow proliferator agent, the method comprising:
   (i) contacting a cancer cell with an agonist of AKT1 degradation;
   (ii) contacting the cell obtained from step (i) with a test agent;
   (iii) determining the anti-tumor effect of the test agent;
   (iv) identifying a test agent as an anti-slow proliferator agent when a statistically significant anti-tumor effect is observed.
18. The method of paragraph 17, wherein the cancer cell is maintained in vitro.
19. The method of paragraph 17, wherein the cancer cell is maintained in vivo.
20. A method comprising;
   (i) obtaining a tumor biopsy from a subject;
   (ii) determining the expression level of TTC3 in cells obtained from the subject;
   (iii) identifying the presence of slow proliferators in the tumor when cells with increased levels of expression of TTC3 are detected.
21. The method of paragraph 20, wherein the expression level of TTC3 and optionally Hes1, AKT1; H3K9me2; MCM2; MK167; CDC6; GMNN; AURKA; PLK1; H3S10ph; H3K4me2; H3K9me2; H3K27me3; H4K12ac; or H4K16ac are determined; and
   wherein the presence of slow proliferators in the tumor is indicated when cells with increased levels of expression of TTC3 and optionally,
   increased levels of expression of Hes1 or decreased levels of expression of AKT1; H3K9me2; MCM2; MK167; CDC6; GMNN; AURKA; PLK1; H3S10ph; H3K4me2; H3K9me2; H3K27me3; H4K12ac; or H4K16ac are detected.
22. The method of any of paragraphs 20-21, wherein the method further comprises administering an inhibitor of AKT1 degradation to the subject to a subject identified as having cancer cells with increased levels of expression of TTC3.
23. A method of producing slow proliferator cancer cells, the method comprising:
   (i) contacting a cancer cell with an agonist of AKT1 degradation;
   (ii) maintaining the cancer cells treated in step (i).
24. The method of paragraph 23, wherein the method further comprises the step of enriching the cells of step (ii) for slow proliferators by selecting for cells having increased levels of expression of TTC3 and optionally, increased levels of expression of Hes1 or decreased levels of expression of AKT1; H3K9me2; MCM2; MK167; CDC6; GMNN; AURKA; PLK1; H3S10ph; H3K4me2; H3K9me2; H3K27me3; H4K12ac; or H4K16ac.
25. The method of paragraph 23, wherein the method further comprises the step of enriching the cells of step (ii) for slow proliferators by selecting for cells having increased levels of expression of TTC3 and Hes1 and decreased levels of expression of AKT1; H3K9me2; MCM2; MK167; CDC6; GMNN; AURKA; PLK1; H3S10ph; H3K4me2; H3K9me2; H3K27me3; H4K12ac; and H4K16ac.
26. A kit for performing the method of any of paragraphs 20-22.
27. The kit of paragraph 26, wherein the kit comprises a detection agent specific for an expression product of TTC3.
28. The kit of any of paragraphs 26-27, wherein the kit comprises a detection agent specific for an expression product of at least one of the genes selected from the group consisting of: TTC3; Hes1 ; AKT1; H3K9me2; MCM2; MK167; CDC6; GMNN; AURKA; PLK1; H3S10ph; H3K4me2; H3K9me2; H3K27me3; H4K12ac; and H4K16ac.
29. The kit of any of paragraphs 27-28, wherein the detection agent is an antibody reagent.

### EXAMPLES

### Example 1

Human tumors contain rapidly proliferating cancer cells that dictate rate of growth, progression, and response to treatment. However, tumors also have many slowly proliferating cells whose origin and significance are poorly understood. Described herein is the discovery that cancer cells in culture occasionally trigger a fundamental mechanism to divide asymmetrically and produce slow proliferators at low frequency. This mechanism involves mTORC2 signaling during mitosis inducing asymmetric degradation of the AKT1 protein kinase via a TTC3/proteasome-mediated pathway. Inhibiting this mechanism selectively reduces asymmetrically dividing cancer cells and slow proliferators in the population and significantly retards tumor growth in vivo. Conversely, inducing cancer cells to divide asymmetrically and produce slow proliferators markedly increases their tumorigenic potential. These results indicate that asymmetrically dividing cancer cells spawning a small fraction of slow proliferators provide a fundamental advantage for tumorigenesis.

Human tumors are heterogeneous with respect to the fraction of proliferating cancer cells that they contain (1, 2). Tumors with more rapidly proliferating cells clearly grow faster, progress further, and are more difficult to treat (2). But these tumors also contain many slowly proliferating cancer cells that may complicate treatment by resisting cancer therapeutics which preferentially target fast proliferators (3-5). While clonal selection theory clearly explains how rapidly proliferating cancer cells evolve, it remains difficult to understand within this framework why even advanced tumors contain so many slowly proliferating cancer cells (6). Interestingly, slow proliferators can also be found in established human cancer cell lines (7). Cancer cells in culture usually divide to produce two daughters that will divide again in relative synchrony, but occasionally these cells will divide to produce one daughter cell with a markedly slower proliferative rate than the other. Since established cell lines have been grown for many years under experimental conditions that ought to favor purifying selection for a rapidly and uniformly dividing population, this asynchronicity in cell culture is quite puzzling and remains poorly understood. It is generally assumed to simply reflect random variation among individual cancer cells in the many genetic and non-genetic factors that influence transit through the cell cycle (8).

The inventors have discovered that cancer cells divide asymmetrically at low frequency (i.e. <5% of cell divisions) in established lines. These asymmetrically dividing cancer cells produce one rapidly proliferating AKT^{high} daughter cell and another AKT^{low} daughter that down-regulates multiple proliferation proteins and is very slowly cycling (e.g. MK167^{low}, MCM2^{low}, CDC6^{low}, GMNN^{low}) (7). AKT^{low} cells also suppress multiple nuclear histone marks associated with both transcriptional activation and repression, mimicking an epigenomic profile that has been observed in quiescent cell populations (e.g. H3S10ph^{low}, H3K4me2^{low}, H3K9me2^{low}, H3K27me3^{low}). Furthermore, AKT^{low} cells up-regulate HES1, a transcription factor that marks cells that have exited the cell cycle into a G0 state. Since AKT^{low} cells do eventually divide, reverting to an AKT^{high} proliferative phenotype over time, the term “G0-like” is used herein to emphasize the temporary and reversible nature of this cell state. Cancer cells dividing asymmetrically in this way can produce symmetrically dividing progeny and vice versa, suggesting that asymmetric division is not the unique property of a specialized cancer cell subpopulation but rather can be found in any dividing population at equilibrium. Importantly, the inventors have also found AKT^{low} cancer cells within actual human tumors at low frequency where they preferentially survive exposure to combination chemotherapy, suggesting that these slow proliferators may represent an important but unappreciated reservoir of treatment resistance in patients. These intriguing observations prompted led to the question of how this asymmetric cancer cell division is regulated.

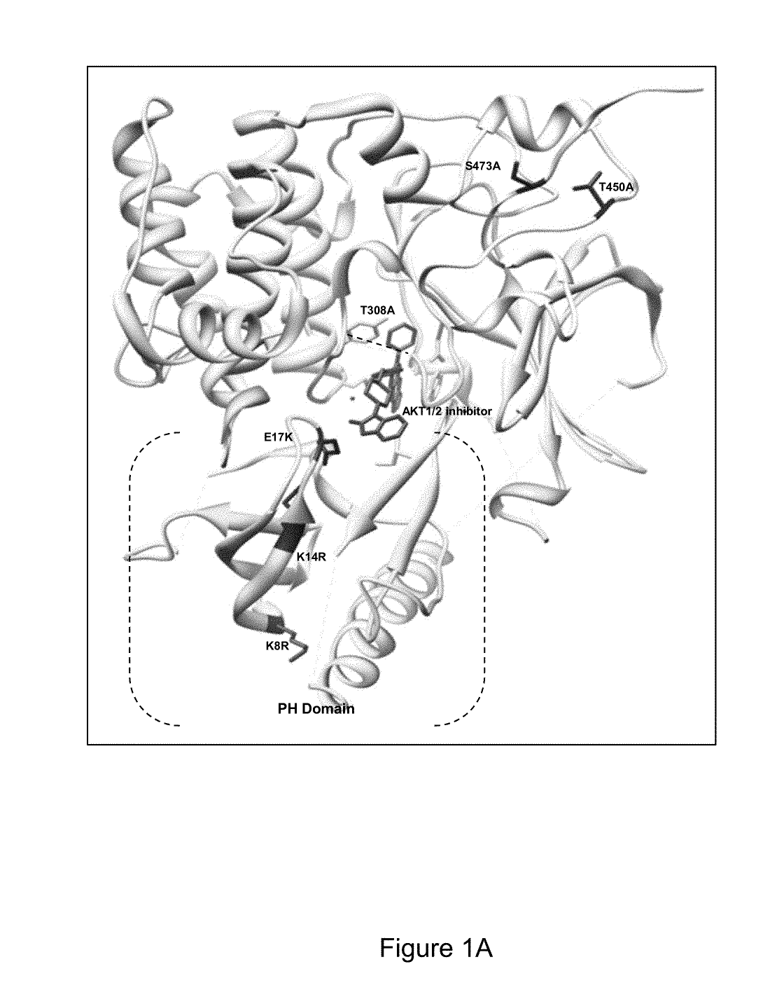

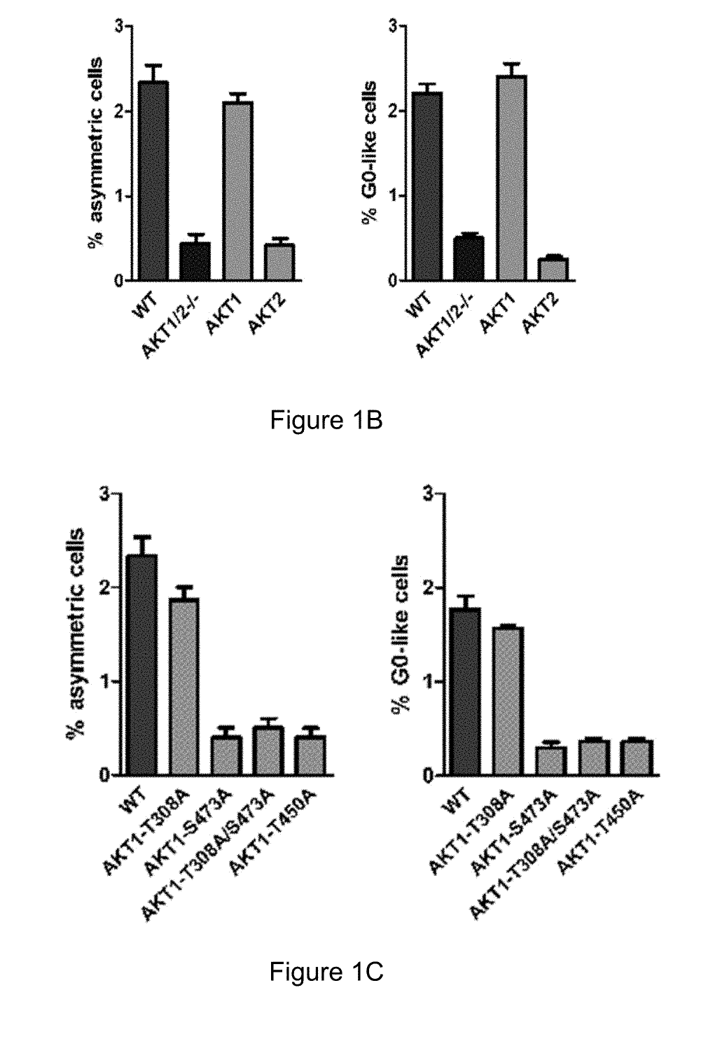

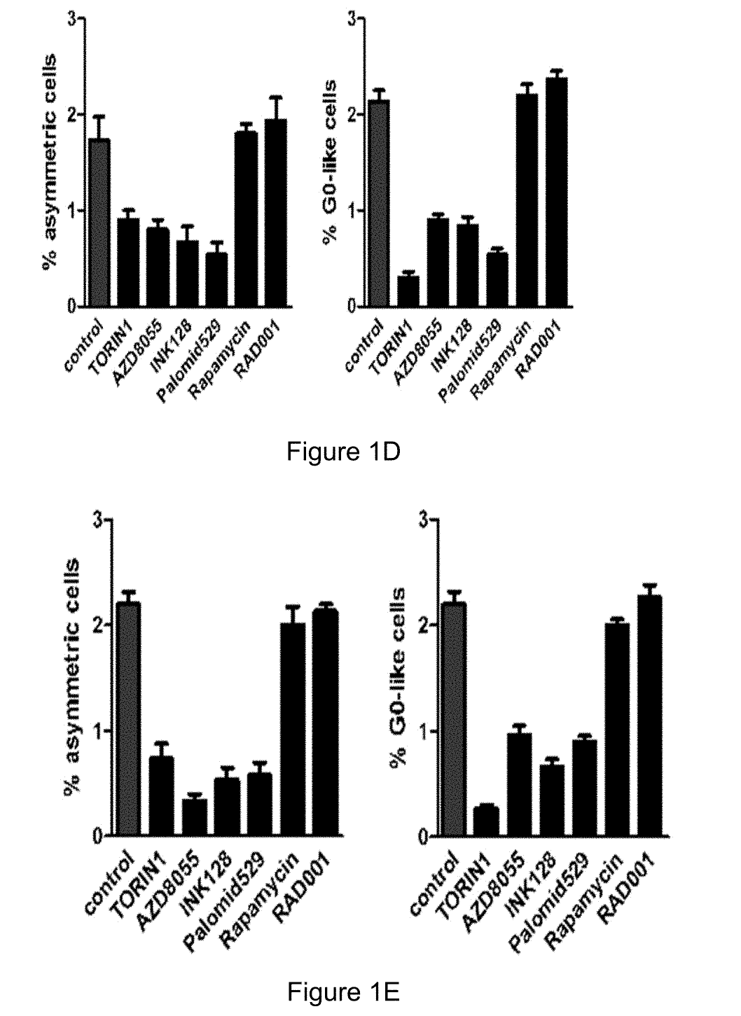

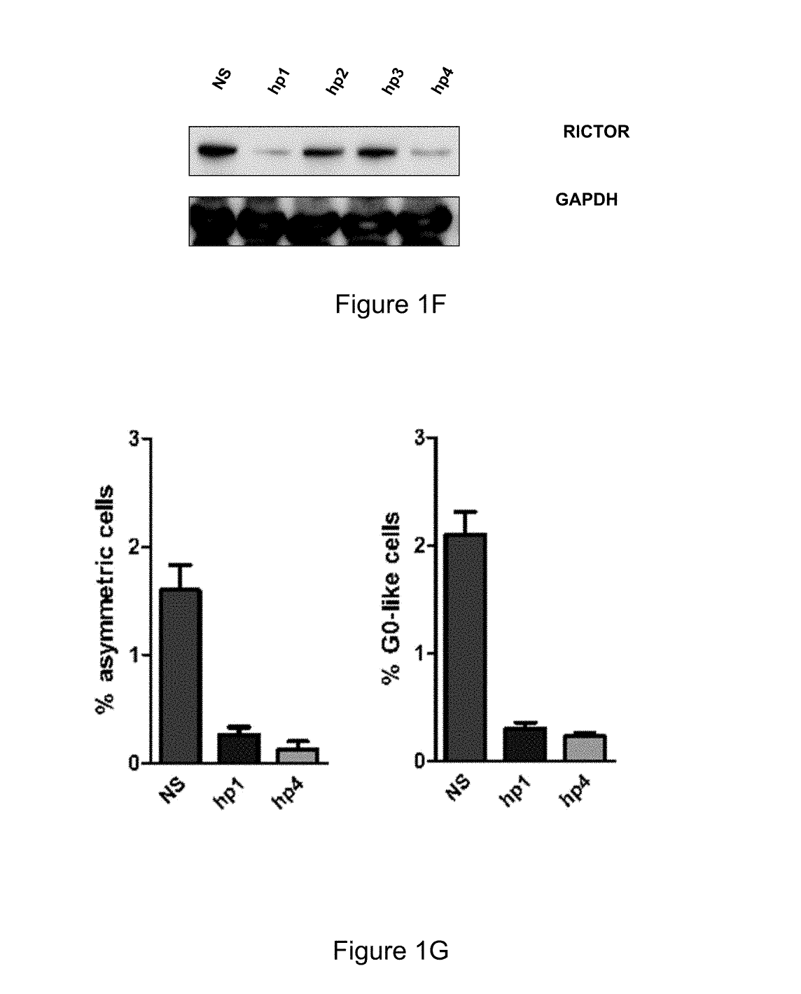

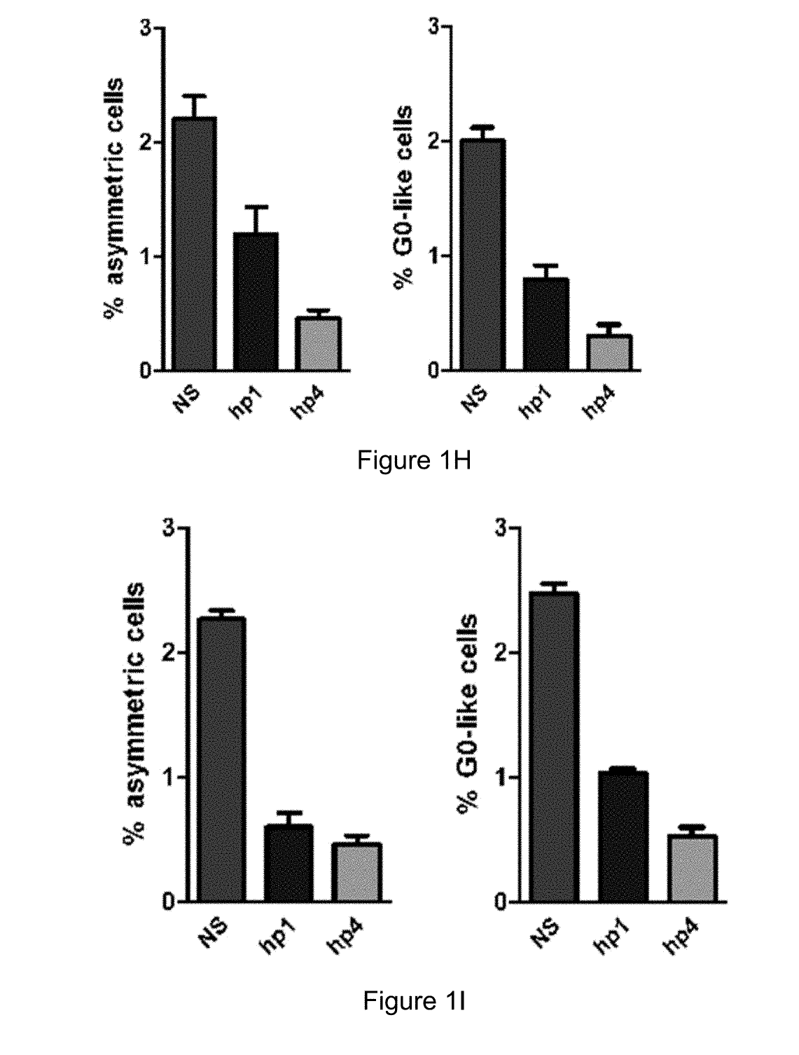

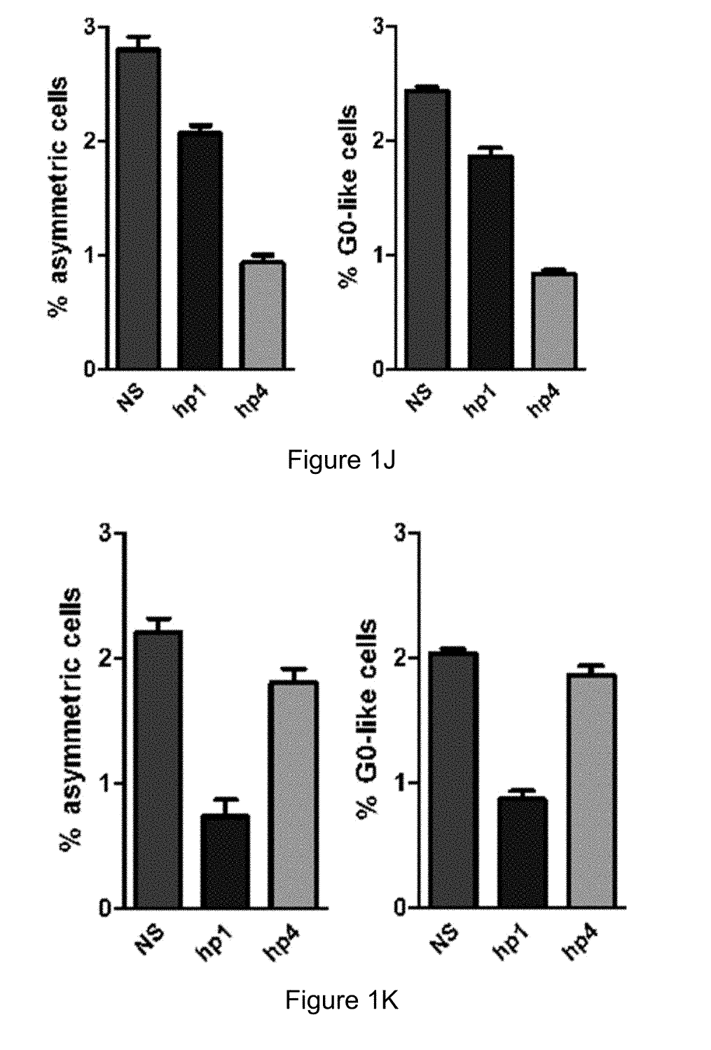

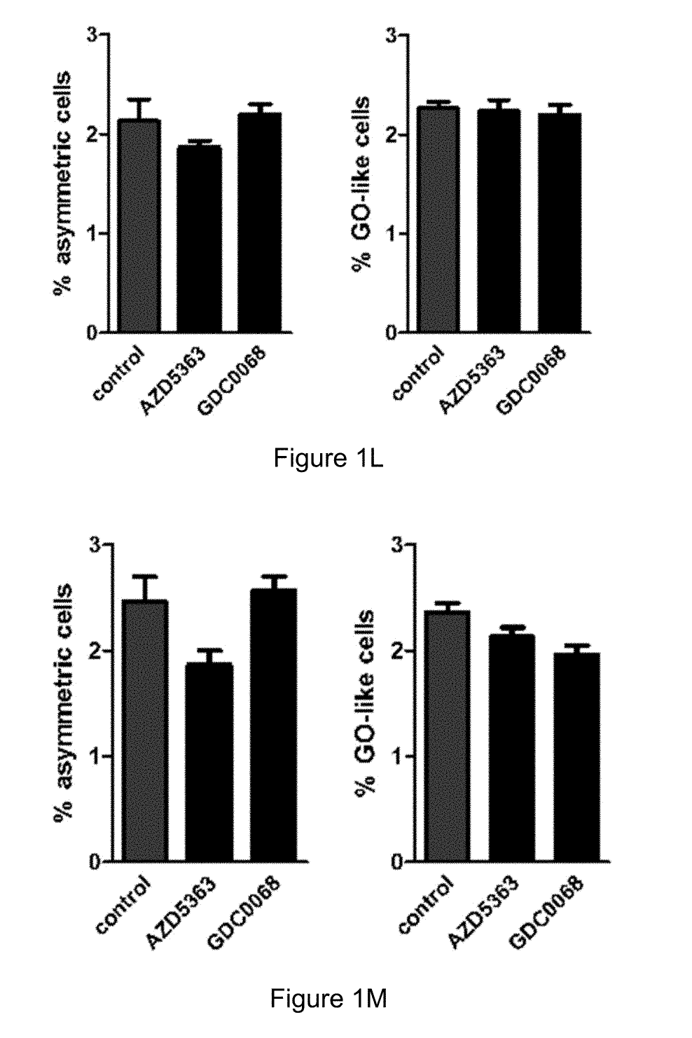

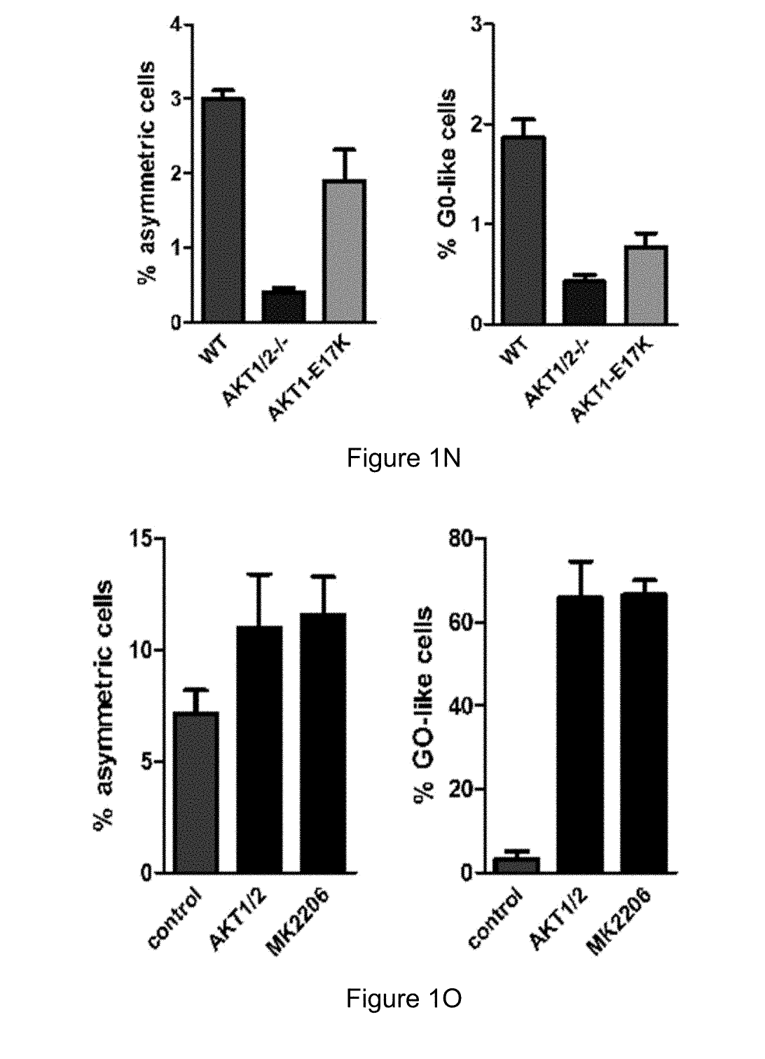

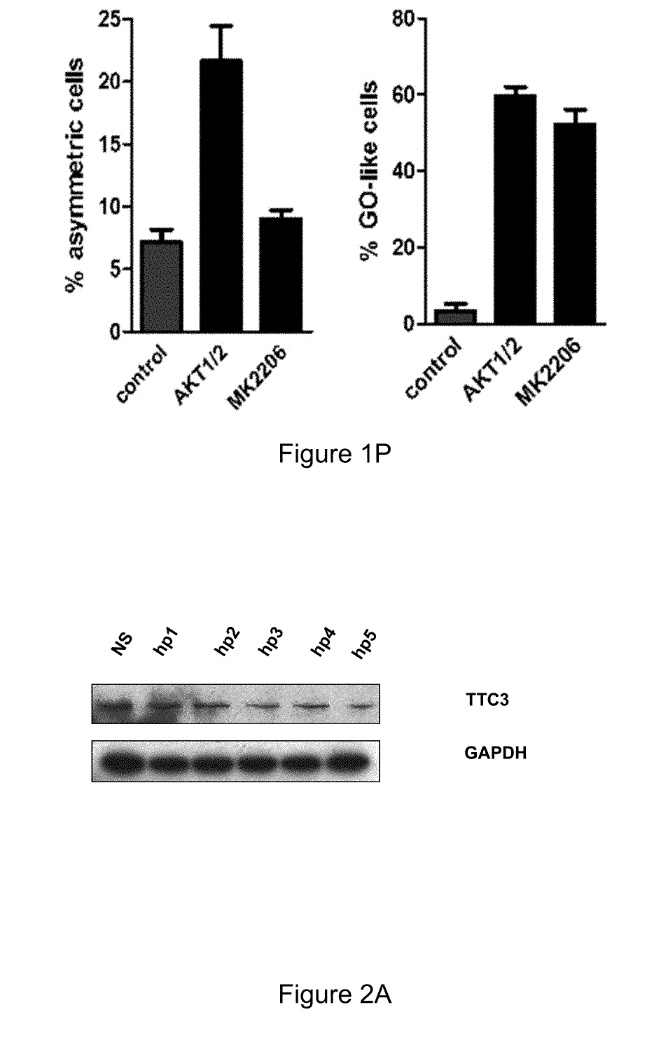

Since AKT^{low} cancer cells partially suppress AKT protein levels (by about 90%), it was first asked whether asymmetric cancer cell division occurs in the complete absence of AKT protein (FIG. 1A) (7). HCT116 colorectal cancer cells with adeno-associated virus (AAV)-mediated disruption of the AKT1 and AKT2 gene loci (i.e. AKT1/2^{-/-} cells) were obtained (9). Importantly, AKT1/2^{-/-} cells do not have AKT1 or AKT2, nor do they express AKT3, but they are able to survive and proliferate in the complete absence of AKT signaling, presumably through compensatory changes that arose during their initial selection. Confocal microscopy was used to score the AKT1/2^{-/-} cell line for rare, asymmetrically dividing and G0-like cancer cells that express the previously validated MCM2^{low}/H3K9me2^{low}/HES1^{high} marker profile. Interestingly, it was found that the AKT1/2^{-/-} line had virtually no asymmetrically dividing or G0-like cells compared to wild type HCT116 (FIG. 1B). In addition, lentiviral-mediated overexpression of an AKT1 cDNA in AKT1/2^{-/-} cells completely rescued production of both asymmetrically dividing and G0-like cells, while overexpression of AKT2 did not (FIG. 1B). These intriguing results indicated that AKT1 is both necessary and sufficient for asymmetric cancer cell division and the production of G0-like cells.

The identification of an upstream pathway that might suppress AKT1 protein levels during cell division was undertaken. Site-directed mutagenesis was used to create a series of AKT1 cDNA constructs with mutations in critical amino acids known to be important for different aspects of AKT1 signaling (FIG. 1A). Each mutant AKT1 construct was then overexpressed in AKT1/2^{-/-} cells and these engineered cells scored for both asymmetrically dividing and G0-like cancer cells. Two different upstream signaling pathways are known to activate the AKT1 protein kinase through phosphorylation: the PDPK1 kinase phosphorylates AKT1 at the T308 residue while the mTORC2 kinase complex phosphorylates the AKT1-5473 and AKT1-T450 sites (10, 11). It was asked whether either of these canonical AKT1 residues were necessary for asymmetric cancer cell division. Similar to wild-type AKT1, overexpression of the AKT1-T308A mutant (which cannot be phosphorylated by PDPK1) in AKT1/2^{-/-} cells completely restored the production of asymmetrically dividing and G0-like cells (FIG. 1C). However, overexpression of AKT1-S473A or AKT1-T450A (which cannot be phosphorylated by mTORC2), or an AKT1-T308A/AKT1-S473A double mutant, did not produce this phenotypic rescue (FIG. 1C). These results indicated that mTORC2 signaling can induce asymmetric cancer cell division.

To test this hypothesis, two complementary approaches were used to disrupt mTORC2 signaling and changes in the frequency of asymmetrically dividing and G0-like cells were then scored. First, it was found that four structurally-different small molecules that inhibit both mTORC1 and mTORC2 signaling significantly reduced the frequency of asymmetrically dividing and G0-like cells in both HCT116 and MCF7 breast cancer cells (i.e. TORIN1 AZD8055, INK-128, Palomid-529) (FIG. 1D,1E). In contrast, two different inhibitors that preferentially target the TORC1 signaling complex alone did not suppress production of these cells (i.e. Rapamycin, RAD-001) (FIG. 1D,1E). In addition, inducible shRNA knockdown of RICTOR (an obligate member of the mTORC2 signaling complex) with two different short hairpin RNAs suppressed both asymmetrically dividing and G0-like cells in a panel of human epithelial cancer cell lines with diverse oncogenomic profiles, including those with a functional dependency on driver mutations in the PI3K signaling pathway (i.e. HCT116 (PIK3CA^{mutant}), MCF7 (PIK3CA^{mutant}) MDA-MB-231 breast, PC9 lung, and A375 melanoma) (FIG. 1F-1K) (10, 12). These findings indicated an important and general role for mTORC2 signaling in triggering rare cancer cells to divide asymmetrically and produce slow proliferators.

It was also asked whether increases in AKT1 signaling would induce asymmetric cancer cell division. However, it was found that two different small-molecules that inhibit AKT1 kinase catalytic activity did not reduce the frequency of asymmetrically dividing or G0-like cells in HCT116 or MCF7 (i.e. AZD5363, GDC0068) (FIG. 1L,1M). Furthermore, AKT1-E17K (a variant oncogenic protein with constitutive enzymatic activity resulting from a somatic point mutation in the kinase domain) partially rescued but did not significantly increase asymmetrically dividing or G0-like cells compared with AKT1 in the AKT1/2^{-/-} line (FIG. 1N) (13). This indicated that AKT1 kinase activity itself most likely did not induce asymmetric cancer cell division. In striking contrast, it was found that two different allosteric (rather than catalytic) inhibitors of AKT1 at low doses dramatically increased the frequency of both asymmetrically dividing and G0-like cells in HCT116 and MCF7 (i.e. AKT1/2, MK2206) (FIG. 1O,1P). Unlike catalytic inhibitors, these allosteric inhibitors bind to the AKT1 pleckstrin homology domain, displacing the protein from the cell membrane, and inducing its ubiquitination and proteasome-mediated degradation (14). These results indicate that asymmetric cancer cell division depends on the targeted degradation of AKT1 protein.

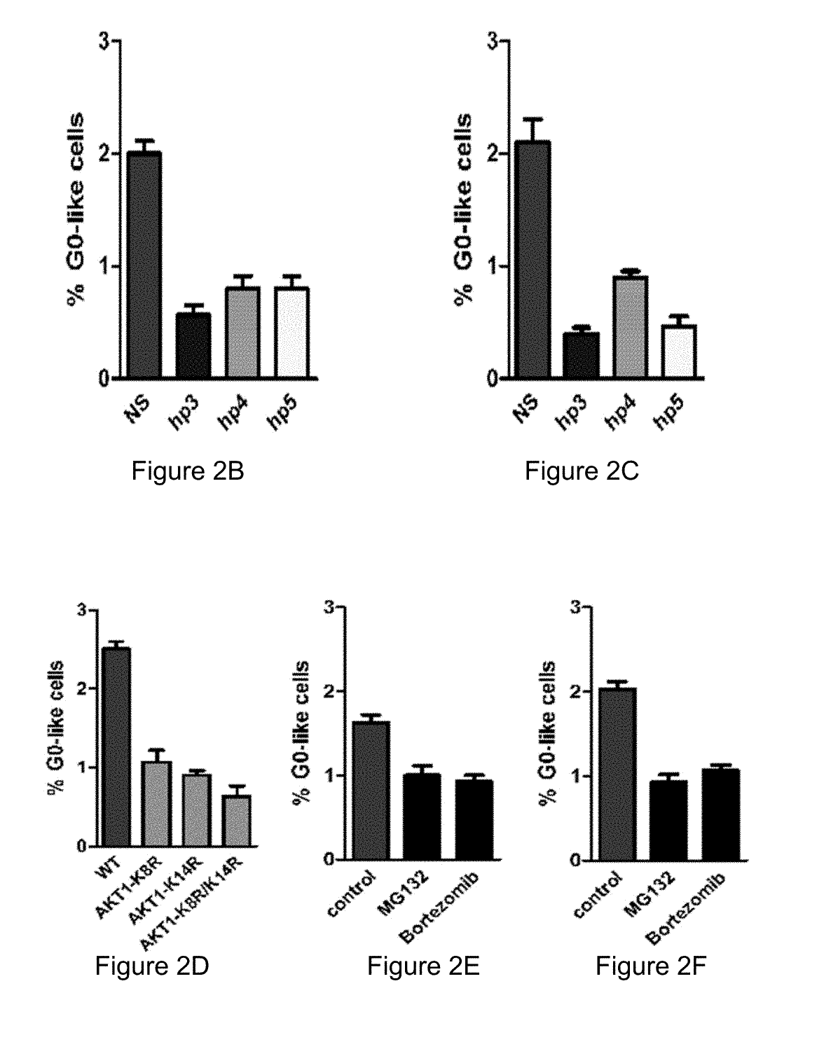

TTC3 is a RING-type E3 protein-ligase known to ubiquitinate AKT1 at the lysine-8 and lysine-14 residues to trigger its destruction by the proteasome (15). Interestingly, it was found that G0-like cells express high levels of TTC3 protein compared to proliferating cells, suggesting that this E3 ligase might play a special role in the production of these slowly cycling cells (data not shown). Consistent with this hypothesis, inducible shRNA knockdown of TTC3 with three different short hairpin RNAs dramatically suppressed the frequency of G0-like cells in both HCT116 and MCF7 (FIGS. 2A-2C). In addition, AKT1-K8R, AKT1-K14R, and AKT1-K8R/K14R double mutant proteins (which cannot be ubiquitinated by TTC3) did not rescue G0-like cells in the AKT1/2^{-/-} line (FIG. 2D). Furthermore, two different small molecules that inhibit proteasome function significantly reduced the frequency of G0-like cells in both HCT116 and MCF7 (i.e. MG-132, Bortezomib) (FIGS. 2E,2F). These results indicated that the production of G0-like cells depends on the TTC3/proteasome-mediated degradation of AKT1.

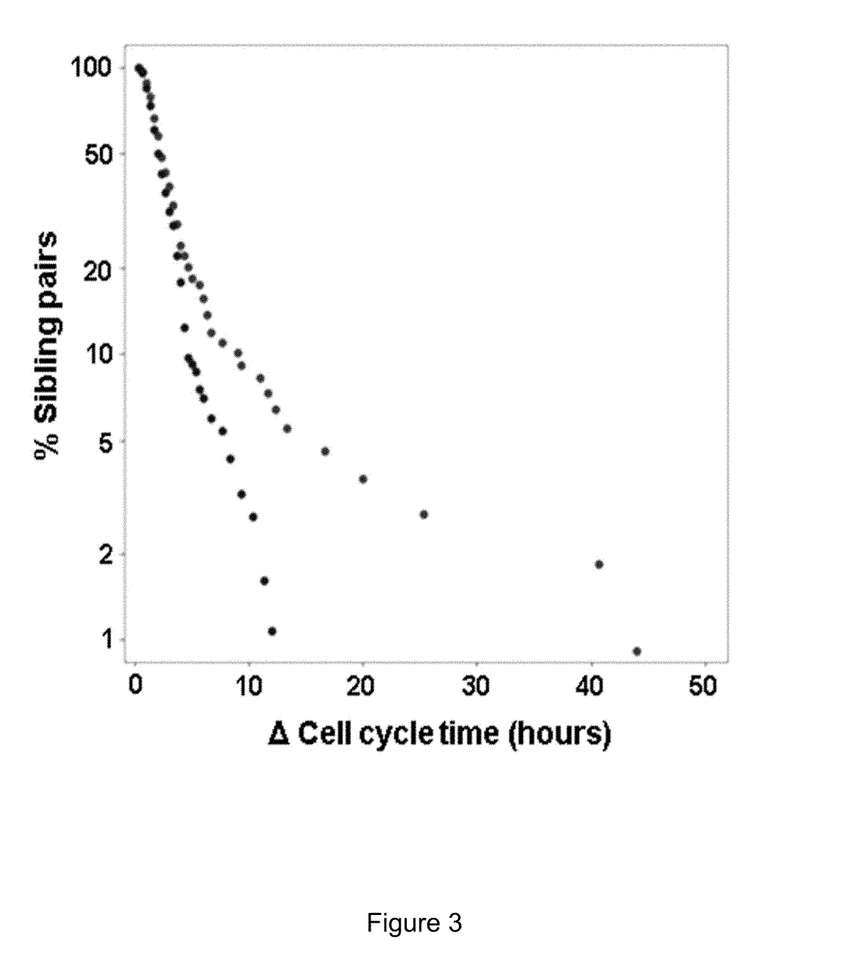

Live-cell imaging experiments were performed to further define the role that mTORC2 signaling plays in regulating asymmetric cancer cell division. Serial images of HCT116 cells dividing over seven days in culture were obtained, either with or without RICTOR knockdown. These images were analyzed to identify individual dividing cells, creating lineage traces of these cells and their progeny to identify sibling pairs, and differences in mitotic times between sister cells arising from the same precursor were plotted. At baseline, ninety percent of dividing cells produced two siblings that divided again within ten hours of each other (FIG. 3). However, approximately ten percent of cells divided more asymmetrically to produce daughters with larger differences in time to mitosis that were greater than ten hours. Remarkably, inducible shRNA knockdown of RICTOR abrogated this minority fraction of most asymmetrically dividing cells, dramatically reducing inter-sibling asynchronicity and proliferative heterogeneity in the population (FIG. 3). Single cell imaging thus confirmed that mTORC2 signaling specifically regulates asymmetric cancer cell division.

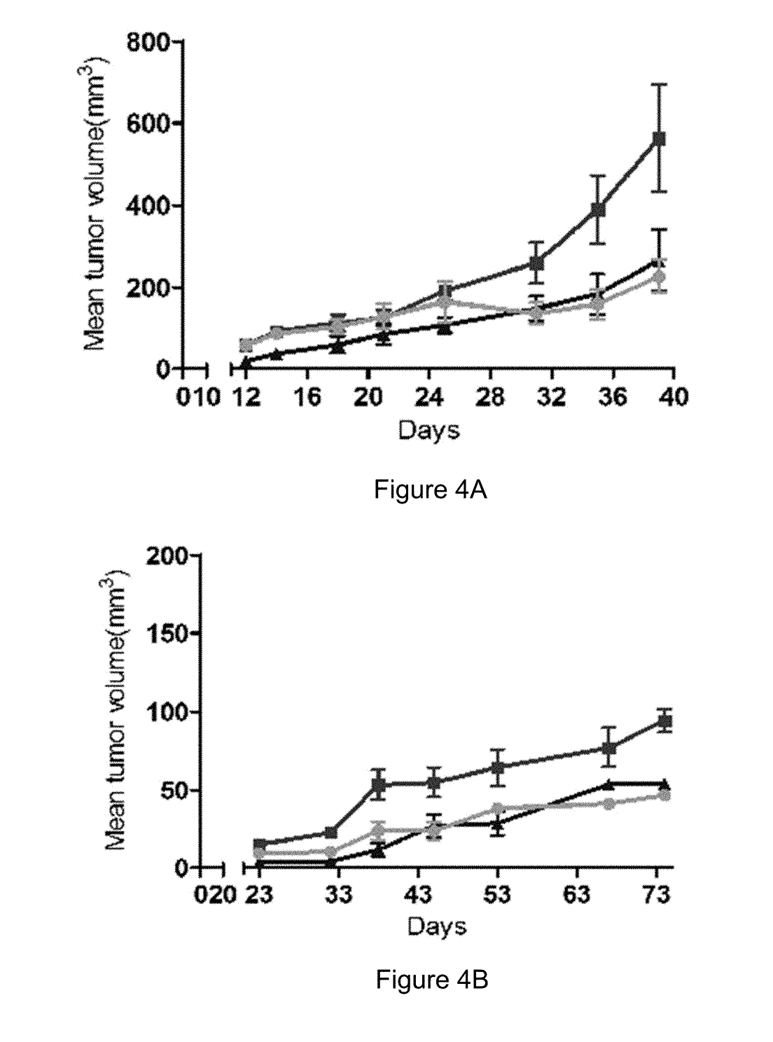

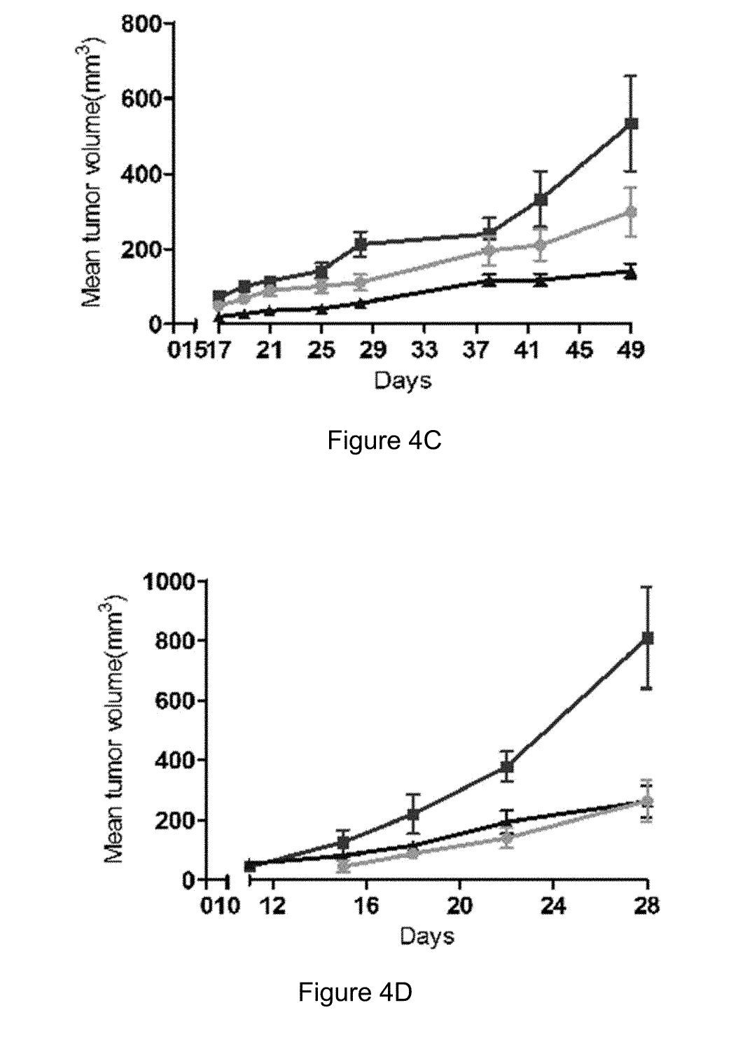

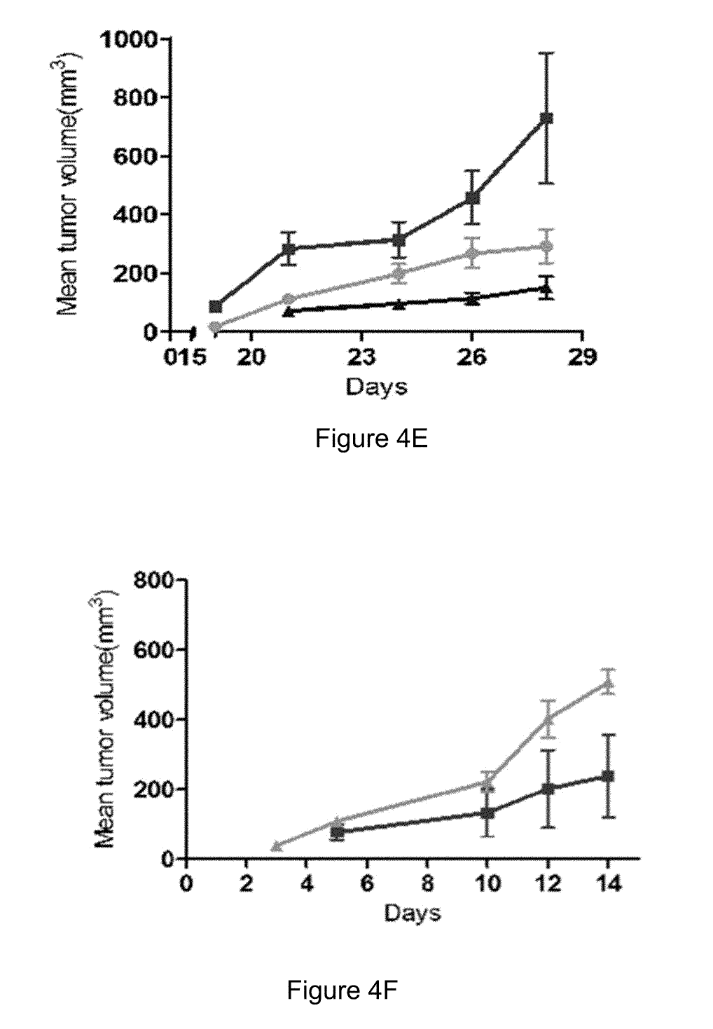

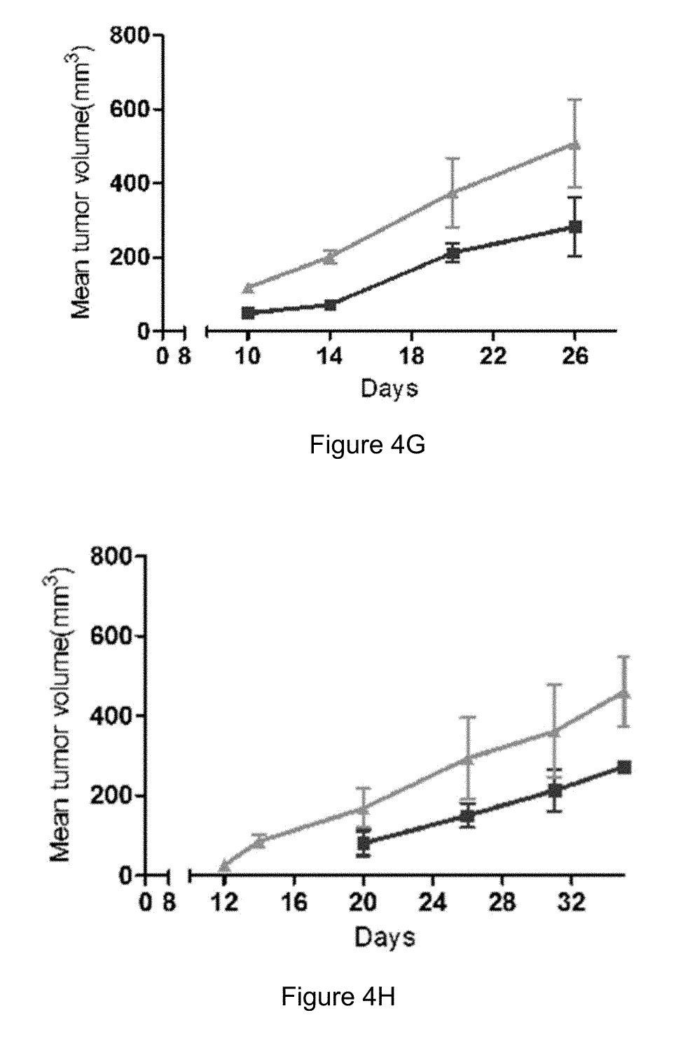

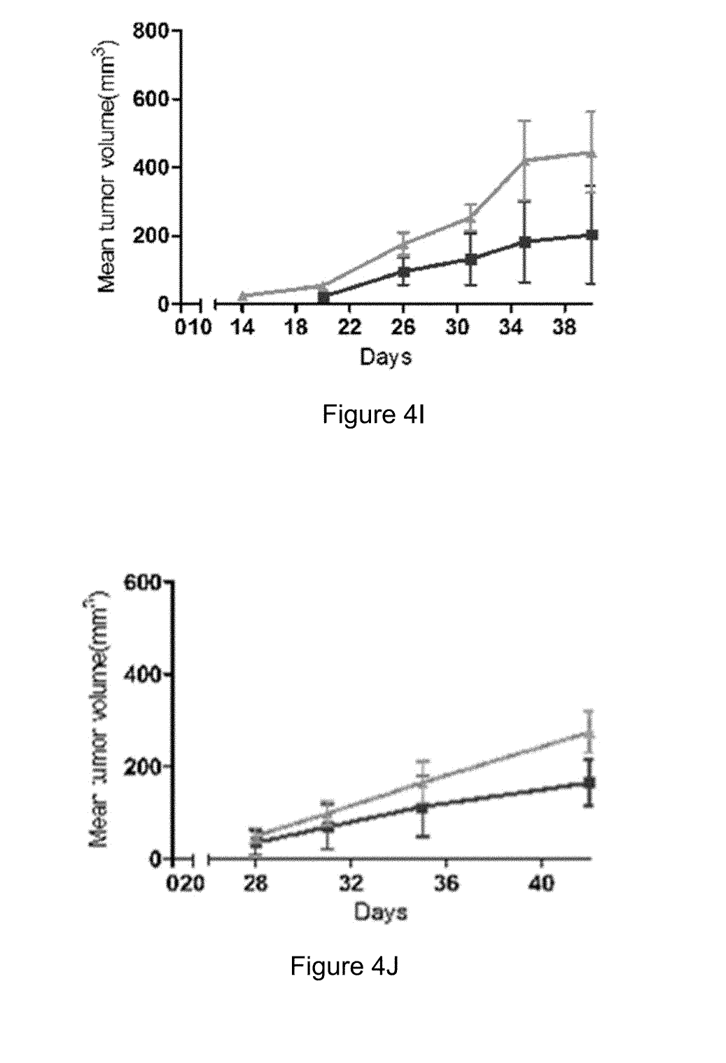

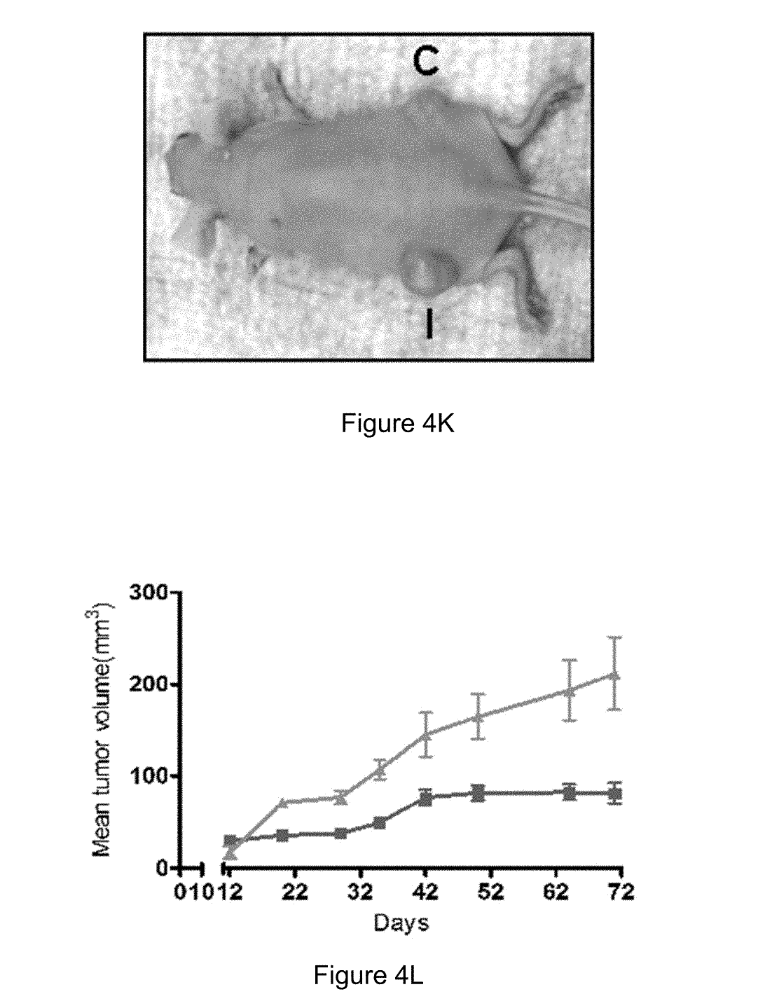

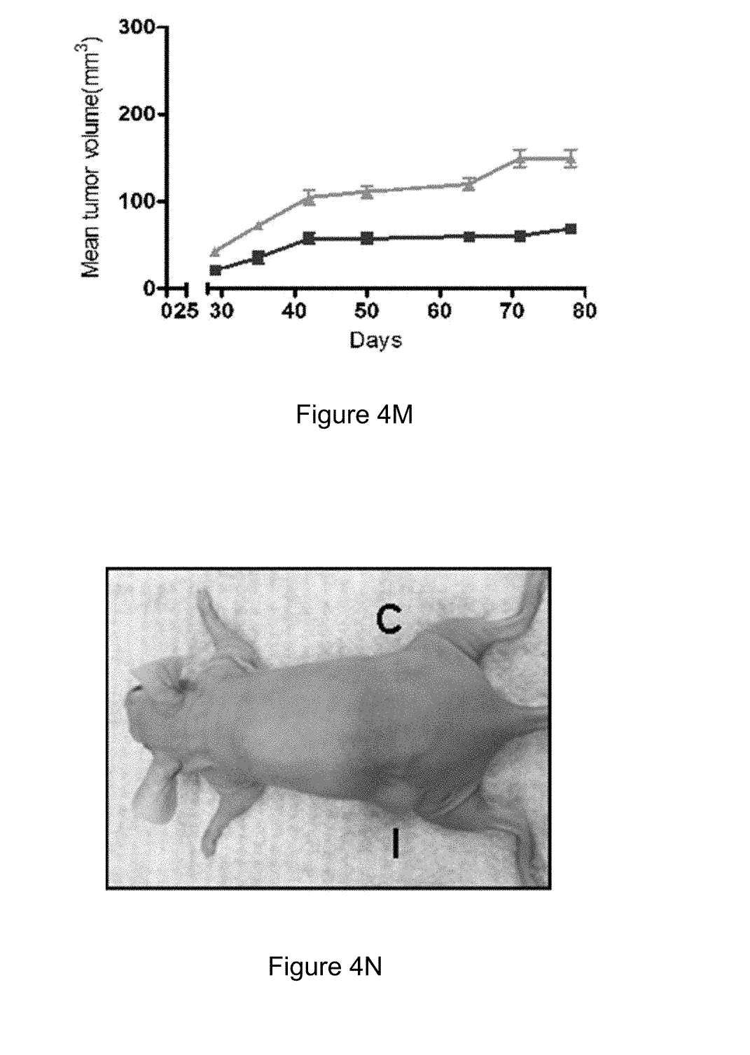

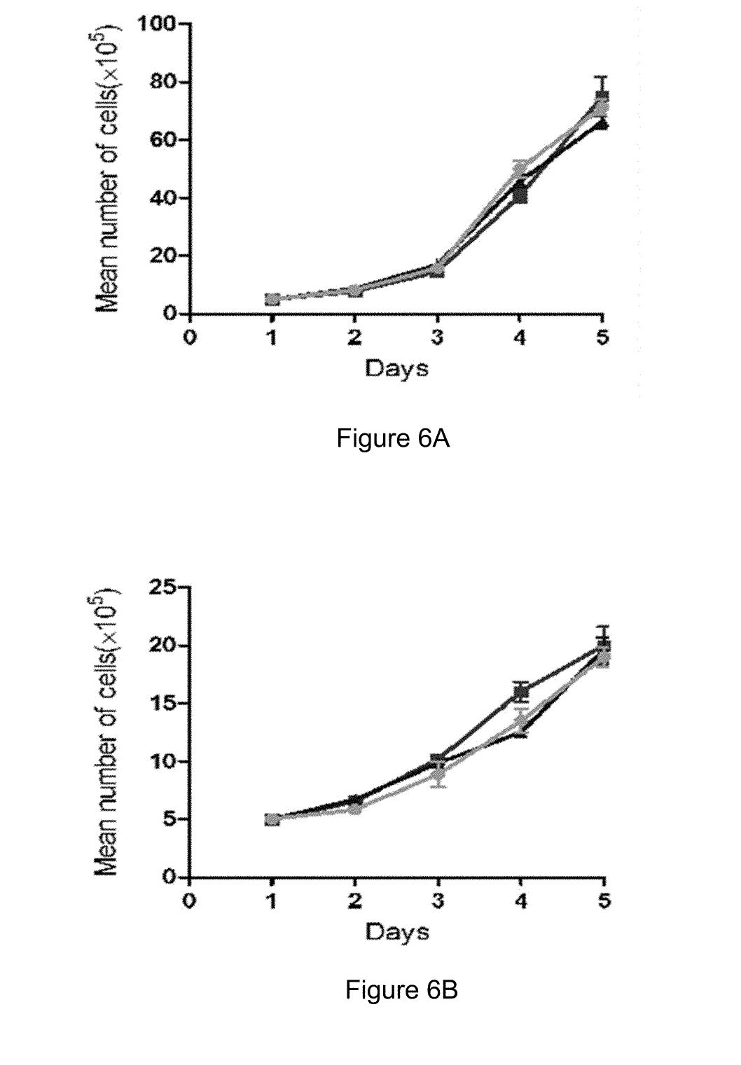

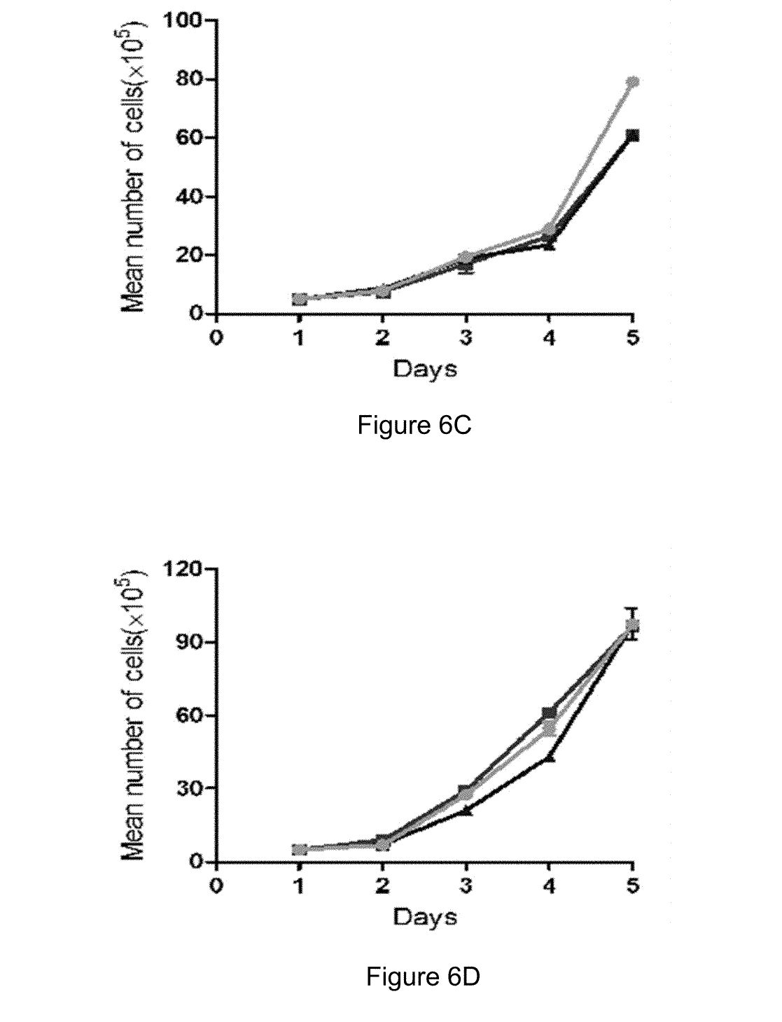

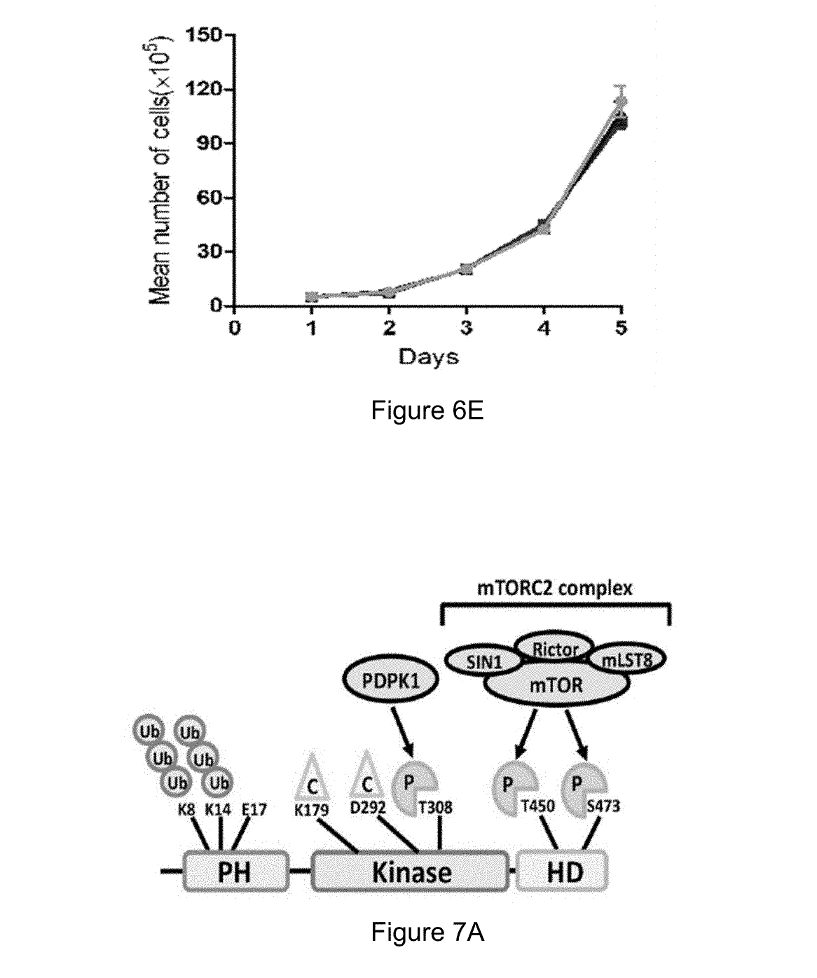

This mechanistic insight provided a unique opportunity to determine the physiological relevance of asymmetric cancer cell division and slow proliferators in growing tumors. Inhibition of mTORC2 signaling through shRNA knockdown of RICTOR clearly inhibited the production of rare asymmetrically dividing and G0-like cells in several human epithelial cancer cell lines (i.e. HCT116, MCF7, MDA-MB-231, PC9, A375) (see FIGS. 1F-1K). However, it was found that disrupting RICTOR did not significantly alter the overall proliferation of these cell lines in vitro (FIGS. 6A-6E). Whether asymmetrically dividing cancer cells might play a special role in tumor formation in vivo was next investigated. These five different cell lines were implanted subcutaneously into immune compromised nude mice. The growth of these lines as xenografts was assessed over several months with or without inducible shRNA knockdown of RICTOR. Remarkably, RICTOR (−) cancer cells with reduced frequency of asymmetric division were markedly less tumorigenic compared to RICTOR (+) cells across the cell line panel, resulting in tumors with that were 50 to 80% smaller in size (FIGS. 4A-4E). These results suggested that the ability of cancer cells to divide asymmetrically and produce small numbers of slowly proliferating progeny significantly enhanced tumorigenesis.

Conversely, it was also asked whether experimentally inducing asymmetric division would increase the tumorigenic potential of cancer cell lines. HCT116 and MCF7 cells were pre-treated for 72 hours with low doses of an allosteric AKT inhibitor (i.e. AKT1/2), which had been found to induce a large fraction of asymmetrically dividing and G0-like cancer cells (see FIGS. 1O,1P). Variable numbers of these pre-treated cells were implanted into nude mice and grown without further manipulation in vivo. Importantly, inducing asymmetric cancer cell division and slow proliferators in this way significantly increased the engraftment of both HCT116 and MCF7 cells over a wide range of initial cell number in vivo, producing tumors that were 50% larger on average than those produced by untreated cell line populations (FIGS. 4F-4N). This was particularly interesting since this AKT inhibitor generally kills cancer cells when used at higher doses (7). This striking finding thus provided further support that G0-like slow proliferators can confer on cancer cell populations a significant selective advantage for growth in vivo.

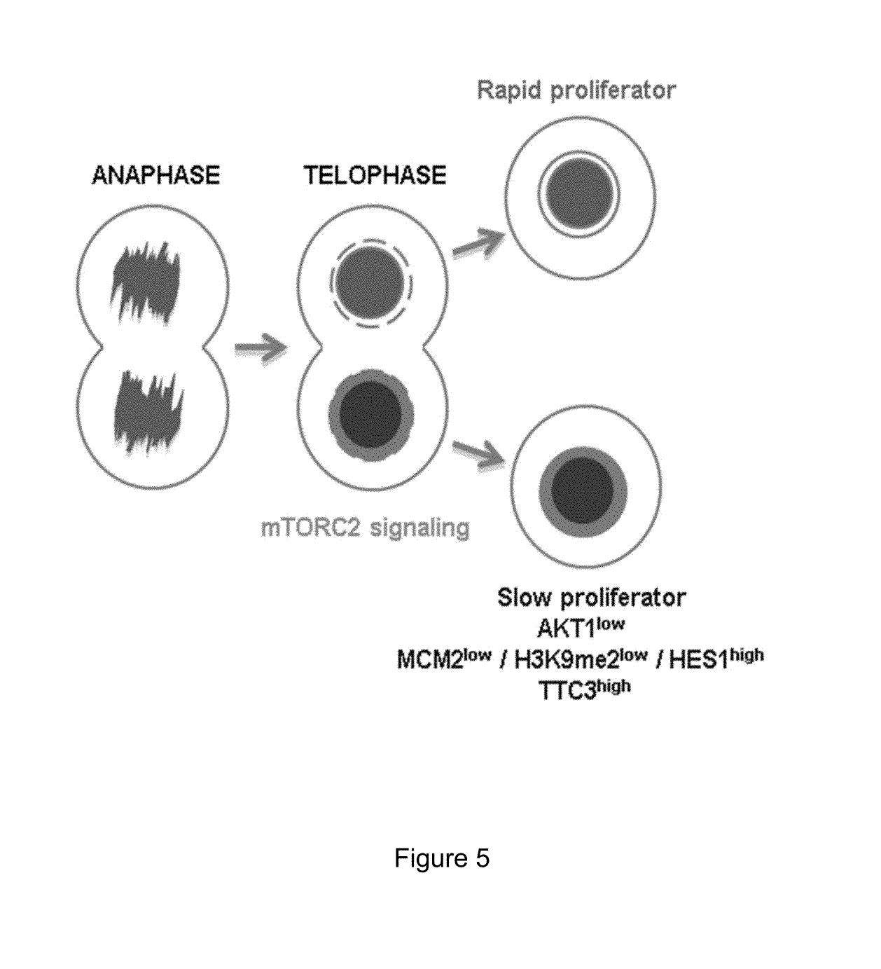

The data presented herein indicate that rapidly dividing cancer cells in culture occasionally trigger an mTORC2 signaling event during mitosis to induce the asymmetric degradation of AKT1 via a TTC3-proteasome-mediated mechanism (FIG. 5). This mechanism continually produces a small fraction of AKT1^{low} proliferators in the population that, remarkably, appears to promote tumor formation in vivo. The mTORC2 signaling complex has been studied extensively over the past decade and is known to be required for tumorigenesis in certain contexts although the exact reasons are unclear (10, 16). The findings described herein suggest that this highly conserved complex may actually regulate a fundamental balance between symmetric and asymmetric cancer cell division that dictates the production of slow proliferators that influence the rate of tumor growth. Without wishing to be bound by theory, it seems unlikely that cancer cells divide asymmetrically in this way simply to promote tumorigenesis. First, cultured cells have a small probability of dividing asymmetrically without deriving obvious advantage in vitro. Second, slow proliferators arising through asymmetric division assume a different, G0-like cell state with global changes in transcription, epigenomic profile, signaling, and metabolic activity compared to their highly proliferative siblings (7). Without wishing to be bound by theory, it is possible that asymmetric cancer cell division may actually represent the execution of a novel cell cycle decision that involves mTORC2 signaling at a very specific point in late mitosis. In this view, the newborn cancer cell with suppressed AKT1 signaling assumes special characteristics (including a slowed cell cycle and G0-like features) that enable it to withstand harsh negative selective pressures during tumor formation, upon transplantation, or on exposure to cytotoxic insult. This model will naturally evoke comparisons to prior work describing putative cancer stem cell populations that might be interesting to pursue using the theoretical, experimental, and mechanistic framework described herein (17-22).

### REFERENCES

1. M. Loddo et al., Br J Cancer 100, 959 (Mar. 24, 2009).
2. J. F. Holland, W. K. Hong, American Association for Cancer Research., Holland-Frei cancer medicine 8. (People's Medical Pub. House, Shelton, Conn., ed. 8th), pp. xxv, 2021 p.

3. S. V. Sharma et al., Cell 141, 69 (April 2).
4. K. R. Coser et al., Proc Natl Acad Sci USA 106, 14536 (Aug. 25, 2009).
5. A. Roesch et al., Cell 141, 583 (May 14).
6. P. C. Nowell, Science 194, 23 (Oct. 1, 1976).
7. I. Dey-Guha et al., Proc Natl Acad Sci USA 108, 12845 (August 2).
8. J. Massague, Nature 432, 298 (Nov. 18, 2004).
9. K. Ericson et al., Proc Natl Acad Sci USA 107, 2598 (February 9).
10. D. D. Sarbassov, D. A. Guertin, S. M. Ali, D. M. Sabatini, Science 307, 1098 (Feb. 18, 2005).
11. B. D. Manning, L. C. Cantley, Cell 129, 1261 (Jun. 29, 2007).
12. M. J. Garnett et al., Nature 483, 570 (March 29).
13. J. D. Carpten et al., Nature 448, 439 (Jul. 26, 2007).
14. H. Jo et al., Proc Natl Acad Sci USA 108, 6486 (April 19).
15. F. Suizu et al., Dev Cell 17, 800 (December, 2009).
16. D. A. Guertin et al., Cancer Cell 15, 148 (Feb. 3, 2009).
17. M. Al-Hajj, M. S. Wicha, A. Benito-Hernandez, S. J. Morrison, M. F. Clarke, Proc Natl Acad Sci USA 100, 3983 (Apr. 1, 2003).
18. M. Diehn et al., Nature 458, 780 (Apr. 9, 2009).
19. J. M. Rosen, C. T. Jordan, Science 324, 1670 (Jun. 26, 2009).
20. P. B. Gupta et al., Cell 146, 633 (August 19).
21. T. Reya, S. J. Morrison, M. F. Clarke, I. L. Weissman, Nature 414, 105 (Nov. 1, 2001).
22. J. A. Magee, E. Piskounova, S. J. Morrison, Cancer Cell 21, 283 (March 20).

### Example 2

### Materials & Methods

Cell culture. HCT116 colon, MCF7 breast, MDA-MB-231 breast, A375 melanoma, and PC9 lung cancer cells were purchased from the American Type Culture Collection (ATCC). HCT116 AKT1/2^{-/-} cells were purchased from Horizon Discovery (Cambridge, UK). MCF7 and MDA-MB-231 cells were maintained in DMEM, 10% FCS, 40 mM glutamine, 100 U/mL penicillin, and 100 μg/mL streptomycin. HCT116 and HCT116 AKT1-/AKT2- cells were maintained in McCoy's 5a medium supplemented with 10% FCS, 100 U/mL penicillin, and 100 μg/mL streptomycin. PC9 cells were maintained in RPMI, 25% glucose, 1% sodium pyruvate, 100 U/mL penicillin, and 100 μg/mL streptomycin. A375 cells were maintained in DMEM supplemented with high glucose HEPES buffer, 10% FCS, 100 U/mL penicillin, and 100 μg/mL streptomycin. All the cells were grown in a humidified atmosphere at 37° C. and 5% CO2.

Drug treatment in vitro. Cells were seeded onto collagen IV-coated coverslips, allowed to attach overnight, and treated with vehicle (DMSO) or AKT1/2 inhibitor (HCT116: 20μM; MCF7: 2 μM) (Sigma), MK2206 (HCT116: 10 μM; MCF7: 3 μM) (Selleck Chemicals), TORIN1 (HCT116: 0.5 μM; MCF7: 0.25 μM) (Tocris Bioscience), AZD8055 (HCT116: 0.7 μM; MCF7: 0.1 μM) (Selleck Chemicals), INK128 (HCT116: 0.05 μM; MCF7: 0.01 μM) (Active Biochem), Palomid 529 (HCT116: 10 μM; MCF7: 20 μM) (Selleck Chemicals), (Rapamycin (HCT116: 20 μM; MCF7: 20 μM)(Sigma), RAD-001 (HCT116: 10 μM; MCF7: 5 μM) (Selleck Chemicals), (AZD5363 (HCT116: 50 μM; MCF7: 5 μM) (Active Biochem), GDC0068 (HCT116: 50 μM; MCF7: 5 μM) (Active Biochem) for 72 h and Bortezemib (HCT116: 1 μM; MCF7: 4 μM) (Selleck Chemicals) MG-132 (vehicle: ethanol) (HCT116: 1 μM; MCF7: 10 μM), for 24 h.

shRNA constructs. Human TRIPZ lentiviral inducible shRNAmirs for Rictor (Clone ID: V2THS_120392, V2THS_120389, V2THS_38014, V2THS_225915), non-silencing, and empty vector were purchased from Open Biosystems and virus was generated using our standard protocol. Infection was performed 24 h later in MCF7, HCT116, A375, PC9 and MDA-MB-231 cell lines with the lentiviral particles followed by selection with 2 μM puromycin. Following selection, cells were allowed to grow to confluency. The shRNAs were induced using 2 μg/ml doxycycline for 72 h. The TTC3 virus was purchased from Sigma-Aldrich and infected in HCT116 and MCF7 cells and the standard protocol for selection was followed.

Generation of AKT1 mutant cell lines. AKT1(WT) cDNA was purified using PCR after cutting PDD AKT1(WT) with restriction enzymes BamHI and Xhol. Following purification, the product was ligated into pMSCVpuro-C-tag-mCherry cut with BglII and SalI. All the AKT1mutants were generated using the QuikChange site directed mutagenesis kit (Agilent technologies) and the product was ligated into pMSCVpuro- C-tag-mCherry. The resulting vector pMSCV-puro-AKT1-mCherry was sub-cloned into DH5α competent cells (Invitrogen). Sequencing verification of the fusion product was performed by the MGH DNA Core Facility with primers pMSCV 5′-CCCTTGAACCTCCTCGTTCGACC-3′ (SEQ ID NO: 1) and pMSCV 3′-GAGACGTGCTACTTCCATTTGTC-5′ (SEQ ID NO2). Virus carrying the desired fusion gene was produced by transfecting 293-T cells with target vector pMSCV-puro-AKT1-mCherry and packaging vector pCL-Ampho using the Mirus TranIT-293 transfection reagent and established protocols. Virus was collected 24 h following transfection. Before infection, cells were plated in a six-well plate in DMEM, 10% FCS. Infection was performed 24 h later by adding 0.5 mL DMEM, 10% FCS, 0.5mL pooled virus, and 1 μL 1,000× polybrene per well. A media change was performed the following day and cells were allowed to grow to confluency before splitting into a 10-cm dish and selection with 2 μM puromycin. Following selection, cells were allowed to grow to confluency before clones were selected using single-cell sorting (Becton Dickinson FACSAria II). Single cells were filtered by gating on the brightest 5% of cells in the PETexas red channel and sorted into individual wells of a 96-well plate. Clones were harvested between 14 and 21 d.

Immunofluorescence staining. For bulk populations and for colonies, cells were grown directly on collagen IV-coated coverslips (Sigma). Cells were fixed in 3.7% formalin, permeabilized using 0.1% Triton X-100, and treated with 0.1% SDS. They were blocked in 1% BSA and then incubated with primary antibody (α-H3K9me2 (Abcam); α-MCM2 and α-Tubulin (Cell Signaling), α-Hes1 and α-TTC3 (Abnova)) diluted in blocking solution, washed, and incubated with the respective secondary antibody. Cells were mounted using hard-set mounting media containing DAPI (Vector Laboratories). All secondary antibodies were Alexa Fluor conjugates (488, 555, 568, 633, and 647) (Invitrogen). Immunofluorescence imaging (on a Nikon Eclipse Ti A1R-A1 confocal microscope) and live-cell imaging (on the Nikon Biostation CT platform) were performed as previously described (1).

Generation of a HCT116-mCerulian-tagged cell line. Virus carrying the pMSCV-CMV-NLSmCerulean construct was produced by transfecting 293-T cells plated at 500,000 cells per well in a six-well plate. Twenty four hours later, these cells were transfected with 1 μg target vector pMSCV-CMVNLS-mCerulean, 1 μg packaging vector pCL-Ampho, and 3 μL FuGENE HD mixed with 100 μL reduced serum solution (Opti-MEM; Invitrogen). Virus was collected 24 h following transfection. Before infection with virus, HCT116 cells were plated at 50,000 cells per well in a six-well plate in DMEM, 10% FCS. Infection was performed 24 h later by adding 0.5 mL DMEM, 10% FCS, 0.5 mL pooled virus, and 1 μL 1,000× polybrene per well. A media change was performed the following day, and cells were allowed to grow to confluency before splitting into a 10-cm plate. MCF7/NLS-mCerulean cells were selected using fluorescence-activated cell sorting (Becton Dickinson FACSAria II) and gating on the brightest 5% cells in the Pacific blue channel

Live-cell imaging & time-lapse analysis. In order to follow the fate of HCT-116 cells in vitro, we plated HCT-116 cells tagged with NLS-mCerulean and also a doxycycline-inducible non-silencing or Rictor knockdown shRNA (hp4) construct in glass-bottom 12-well plates (MatTek Product # P12G-1.0-10-F) treated with type IV collagen. Tagged HCT116 cells were plated in 2 μg/ml of doxycycline at a density of 1000 cells per well along with unlabeled HCT-116 cells at a density of 4000 cells per well. All cells were initially grown in McCoy's 5-alpha+10% FCS at t=0. Media changes were performed every day with 2 μg/ml doxycycline. Multi-point serial imaging was performed using an inverted microscope fitted with a tissue culture incubator (Nikon Ti-Eclipse) every 20 minutes at 20×magnification (CFI Plan Apo 20×) for 164 hours. Both phase and fluorescent images were captured. Cells were excited with an LED (Nikon C-HGFI Intensilight HG Illum) and passed through a filter series (Nikon, C-FL CFP and RFP HC HISN Zero Shift Filter Set). All cell division events were tracked manually using the CFP images by recording the following characteristics for each cell: ID based on initial frame of appearance and x/y coordinate, first frame, last frame, origin ID, progenitor IDs, and x/y coordinates for first and last frame, and end method (division, lost in tracking, lost to wash out, or lost to cell death). Analysis was performed using R v2.8.0 (The R Foundation for Statistical Computing, 2008) by analyzing all division events.

Xenografting & tumor propagation in vivo. For in vivo RICTOR knockdown experiments, 5×10⁵ cells (MCF7, HCT116, A375, PC9, MDA-MB-231 cell lines) carrying either doxycycline-inducible non-silencing or RICTOR-targeting shRNAs (120392, 225915) were injected subcutaneously into the flanks of 5-6 week old, female nude mice. The mice were given doxycycline in water at 20 mg/ml for hairpin induction. For induction of asymmetrically dividing cells and slow proliferators, cells were treated with AKT1/2 inhibitor and DMSO (vehicle) for 72 h and were harvested at 60-70% confluence, and then counted and washed twice in PBS and resuspended in 1:1 Media: Matrigel (BD Biosciences). 5×10⁶, 5×10⁵, 5×10⁴, 5×10³ and 5×10² cells respectively were injected subcutaneously into the flanks of 5-6 week old, female nude mice (Nude/Nude) (Charles River Labs). For all experiments, growing tumors were measured weekly by caliper, and mice were killed after the tumor size reached 1 cm³. Mouse experiments were carried out under a Massachusetts General Hospital Institutional Review Board-approved protocol.

### REFERENCES

1. I. Dey-Guha et al., Asymmetric cancer cell division regulated by AKT. Proceedings of the National Academy of Sciences of the United States of America 108, 12845 (Aug. 2, 2011).

### Example 3

### A Mechanism For Slowly Proliferating Cancer Cells that Promote Tumor Growth

Tumor growth is driven by rapidly dividing cancer cells that arise through mutation and natural selection. Clonal selection does not fully explain, however, why established tumors also contain slowly proliferating cancer cells. We now find that if a dividing cancer cell experiences an asymmetric decrease in β1-integrin signaling, it activates mTORC2 kinase signaling which induces degradation of AKT1 kinase through a TTC3/proteasome mechanism, to produce a slowly proliferating AKT1low daughter cell. Remarkably, disrupting this mechanism for slowly proliferating cancer cells impedes tumor growth, while inducing slow proliferators enhances tumor formation, across a spectrum of cancer xenograft models. These results suggest that rapidly proliferating cancer cells retain a mechanism to spawn slow proliferators for selective advantage during tumorigenesis.

A dividing cancer cell generally produces two daughter cells that divide again in relative synchrony within hours of each other in cell culture. Occasionally, however, a cancer cell divides to produce progeny that are asynchronous, with one daughter cell having a markedly slower cell division time, on the order of days, compared to the other. As described above, this asynchronicity relates to cancer cells asymmetrically suppressing AKT protein kinase levels by about ninety percent during mitosis just before cytokinesis. This asymmetry produces one AKThigh daughter cell that rapidly enters the next cell cycle and another AKTlow cell that remains dormant for a more prolonged time before dividing again. Slowly cycling AKTlow cells reduce their production of reactive oxygen species (i.e., ROSlow), down-regulate proliferation proteins (e.g., MKI67low, MCM2low), suppress multiple nuclear histone marks similar to quiescent cell populations (e.g., H3K9me2low), and up-regulate the HES1 transcription factor that may mark exit from the cell cycle into G0 (i.e., HES1high) (1). Since AKTlow cells do eventually divide, converting to an AKThigh proliferative phenotype over time, the term “G0-like” is used to describe this temporary and reversible cell state. It is described herein that AKTlow cancer cells are found within actual human breast tumors where they preferentially survive therapy with combination chemotherapy, suggesting that these cells may constitute an important but unappreciated reservoir of treatment resistance in patients with breast cancer (1). Since AKTlow cells share a number of conceptual features with putative cancer stem cell populations (e.g., asymmetric division, slow cycling, ROSlow, treatment resistance), it was reasoned that understanding in molecular detail how AKTlow slow proliferators arise might provide fundamental insight into the dynamics of tumor growth (1,2).

Given that AKTlow cancer cells only partially suppress total AKT protein levels, it first asked whether asymmetric division occurs in the complete absence of all three AKT isoforms (i.e., AKT1, AKT2, and AKT3). To do so, HCT116 colorectal cancer cells with adeno-associated virus (AAV)-mediated disruption of the AKT1 and AKT2 gene loci (i.e., AKT1/2−/− cells) were obtained (3). Importantly, AKT1/2−/− cells do not express either AKT1 or AKT2, nor do they express AKT3, and thus are able to survive and proliferate in the complete absence of AKT signaling, presumably through compensatory changes that arose during their initial selection. Confocal microscopy was used to score AKT1/2−/− cell populations for rare, asymmetrically dividing and G0-like cancer cells that express the previously validated MCM2low/H3K9me2low/HES1high marker profile (1). Interestingly, it was found that the AKT1/2−/− line had virtually no asymmetrically dividing or G0-like cells compared to wild type HCT116 (the parental line from which AKT1/2−/− cells are derived) (FIG. 1B). In addition, lentiviral-mediated overexpression of an AKT1 cDNA in AKT1/2−/− cells completely restored formation of both asymmetrically dividing and G0-like cells, while overexpression of AKT2 did not (FIG. 1B). These results indicate that AKT1 is necessary and sufficient for asymmetric cancer cell division and the production of G0-like cells.

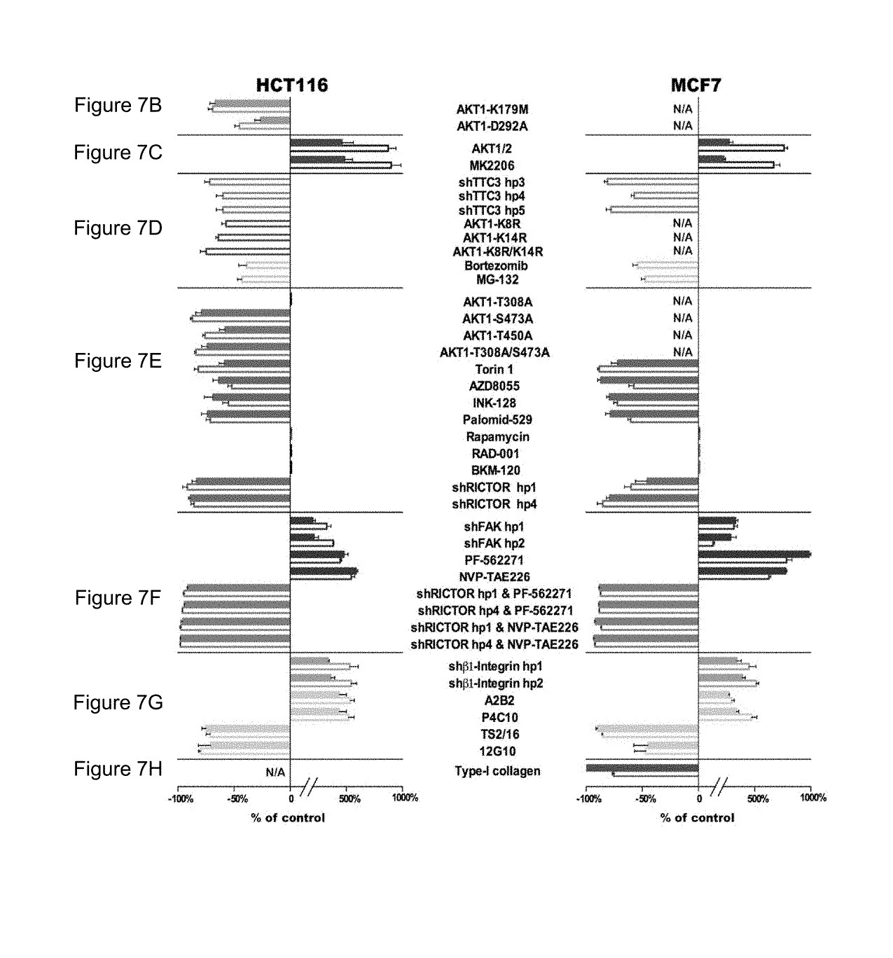

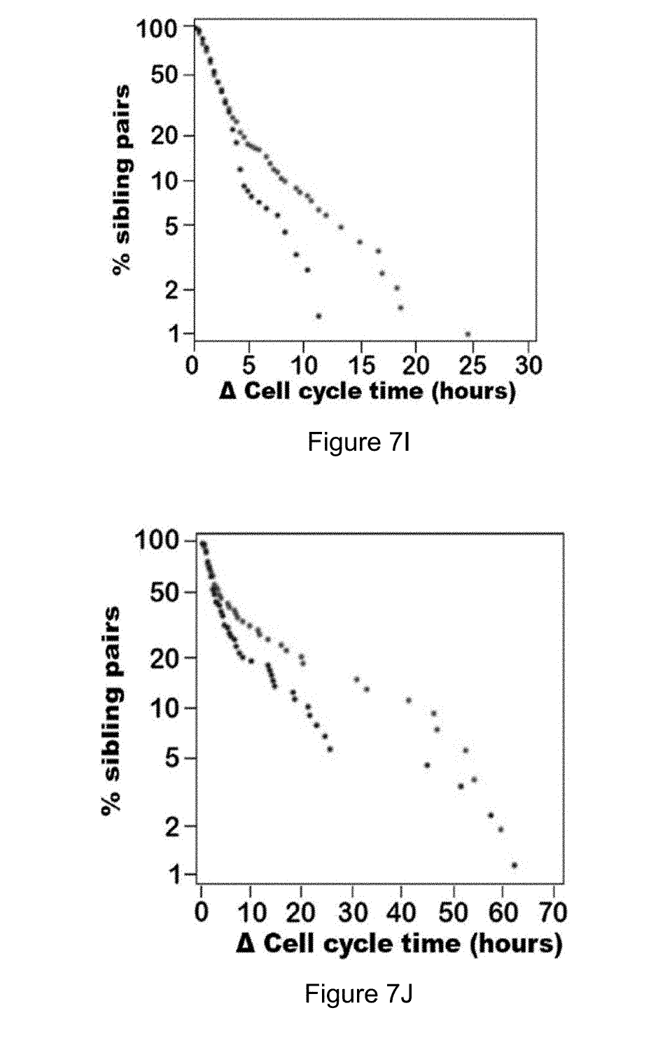

Based on this result, site-directed mutagenesis was used to identify AKT1 domains that might be required for its partial suppression during asymmetric division. A series of AKT1 cDNA constructs with mutations in critical amino acids known to be important for various aspects of AKT1 signaling were created (FIG. 7A). Each mutant AKT1 construct was overexpressed in AKT1/2−/− cells and these engineered cells scored for both asymmetrically dividing and G0-like cancer cells. First, it was found that AKT1-K179M (a mutation in the kinase pocket that renders AKT1 catalytically inactive) failed to restore production of asymmetrically dividing and G0-like cells in the AKT1/2−/− line, while AKT1-D292A (another kinase dead mutant) did so only weakly compared to wild-type AKT1 (FIG. 7B) (4). These results indicated that AKT1 enzymatic activity is necessary for asymmetric cancer cell division.

It was next asked how AKT1 protein is suppressed to produce slow proliferators. As described above, treating cancer cells with allosteric AKT inhibitors at low doses dramatically increases the frequency of both asymmetrically dividing and G0-like cells (i.e., AKT1/2, MK2206) (FIG. 7C) (1). These allosteric inhibitors are known to bind to the AKT1 pleckstrin homology domain, inducing conformational change and displacement of the protein from the cell membrane, promoting its ubiquitination and proteasome-mediated degradation (5). Therefore, it was hypothesized that asymmetric division might actually depend on the targeted degradation of the AKT1 protein. TTC3 is a RING-type E3 protein-ligase known to ubiquitinate AKT1 at its lysine-8 and lysine-14 residues leading to its destruction by the proteasome (6). Interestingly, it was found that G0-like cells express high levels of TTC3 protein compared to proliferating cells, consistent with a potential role for this E3 ligase in producing AKT1low cells (data not shown). In addition, inducible shRNA knockdown of TTC3 with three different short hairpin RNAs suppressed the frequency of G0-like cells in both HCT116 and MCF7 without affecting overall cell proliferation (FIGS. 7D and 2A). Furthermore, AKT1-K8R, AKT1-K14R, and AKT1-K8R/K14R double mutant proteins (which cannot be ubiquitinated by TTC3) failed to rescue the formation of G0-like cells in the AKT1/2−/− line (FIG. 7D). In addition, two different small molecules that inhibit proteasome function reduced the frequency of G0-like cells in both HCT116 and MCF7 when used at doses that do not affect overall cell proliferation (i.e., MG-132, Bortezomib) (FIG. 7D). Overall, these results indicated that AKT1low slow proliferators are produced by TTC3-mediated ubiquitination of AKT1 followed by proteasomal degradation.

Two different upstream signaling pathways are known to activate AKT1 kinase: PDPK1 kinase phosphorylates AKT1 at the T308 residue, while the mTORC2 kinase complex phosphorylates the AKT1-S473 and AKT1-T450 sites (7,8). It was therefore asked whether any of these canonical AKT1 residues were necessary for asymmetric cancer cell division. Similar to wild-type AKT1, overexpression of the AKT1-T308A mutant (which cannot be phosphorylated by PDPK1) in AKT1/2−/− cells completely restored the production of asymmetrically dividing and G0-like cells (FIG. 7E). In contrast, AKT1-S473A, AKT1-T450A, and an AKT1-T308A/AKT1-S473A double mutant (all of which cannot be phosphorylated by mTORC2) did not produce phenotypic rescue (FIG. 7E). These results indicated that mTORC2 signaling might induce asymmetric division by partially phosphorylating and activating AKT1.

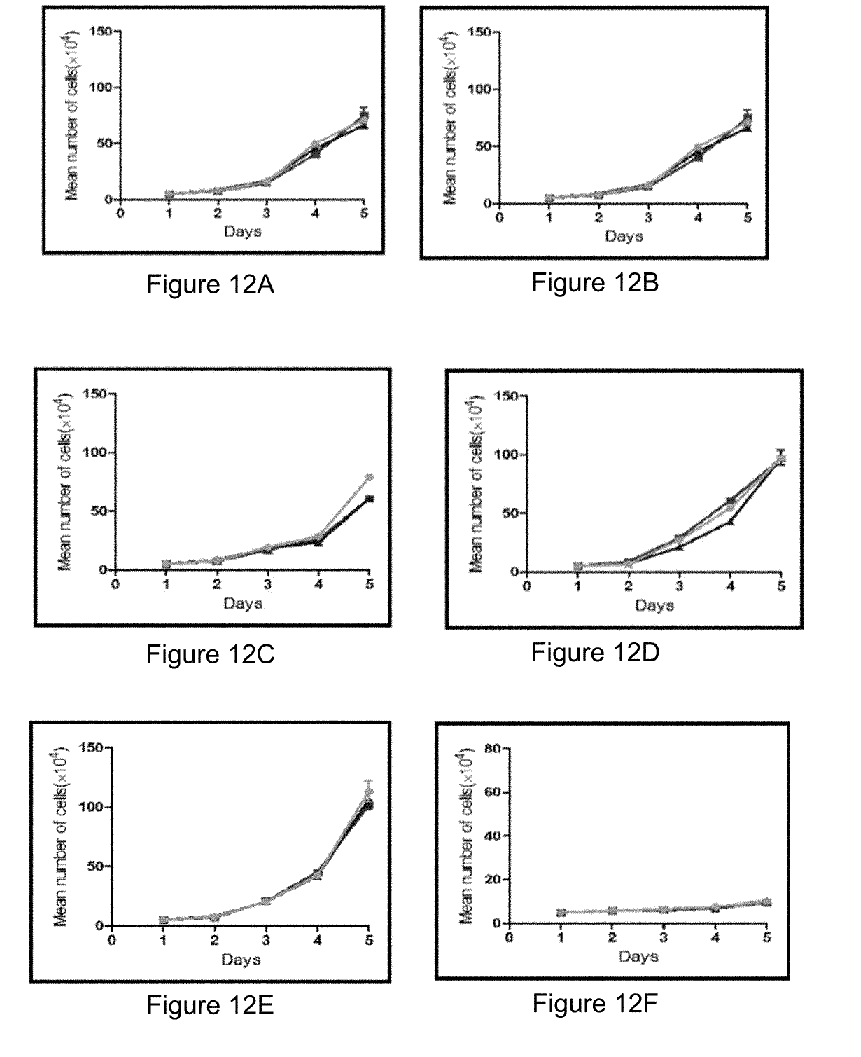

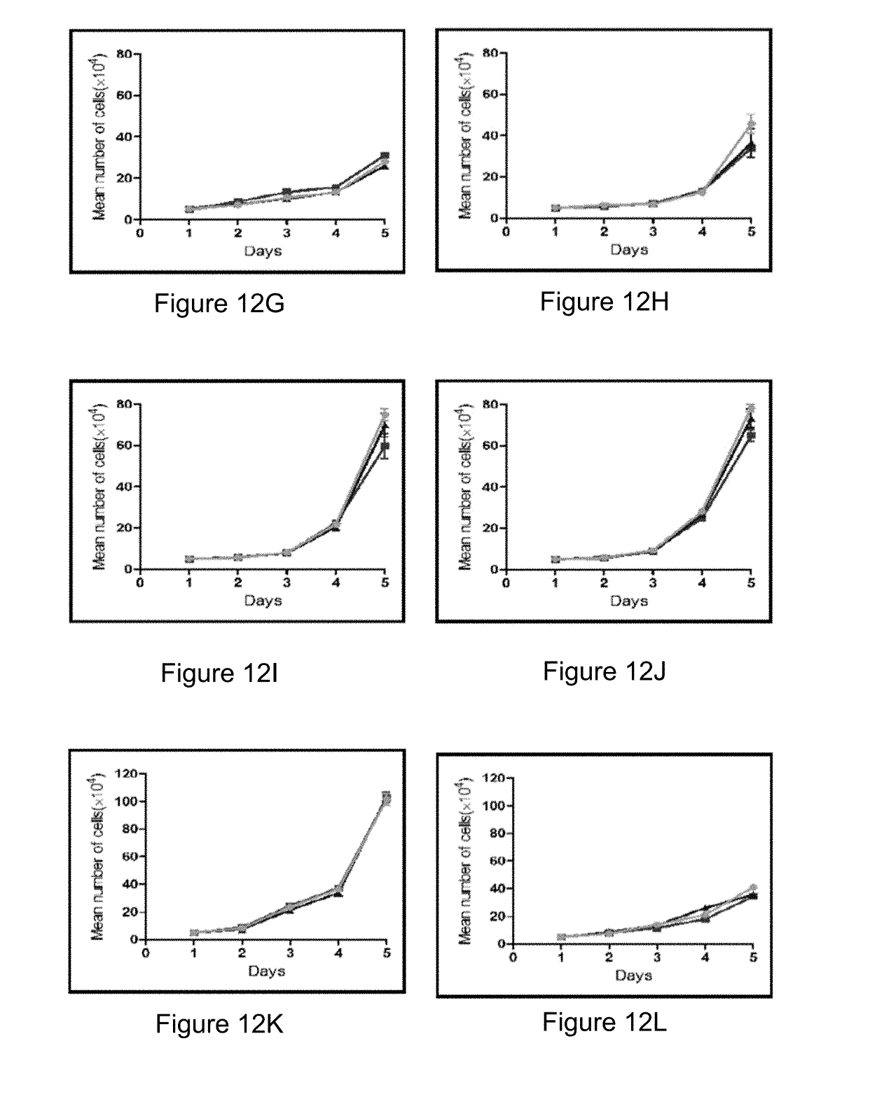

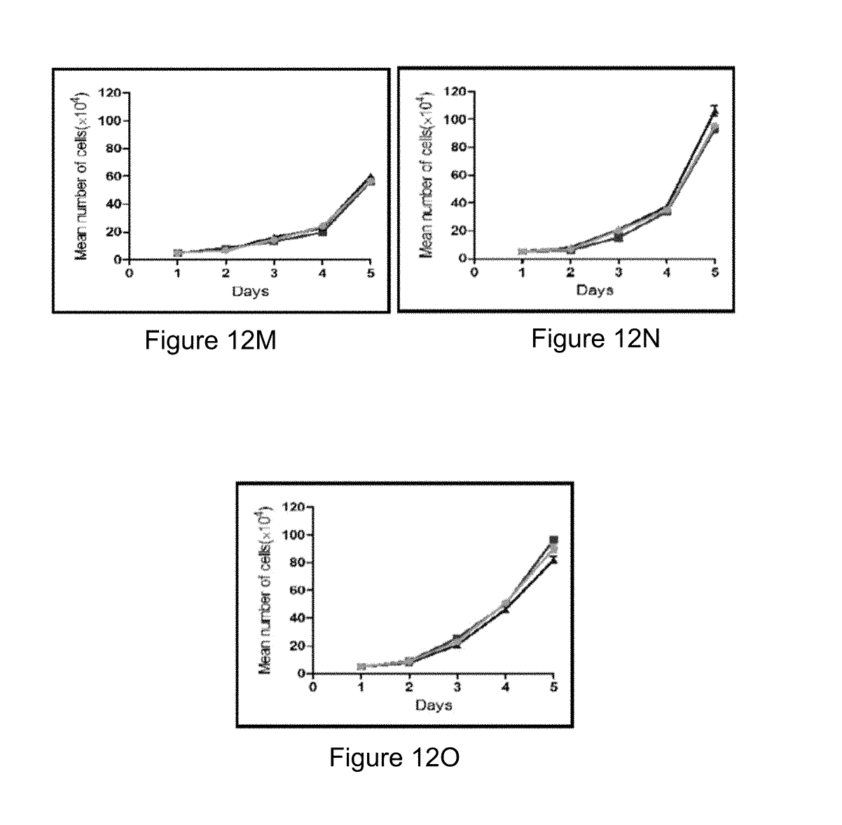

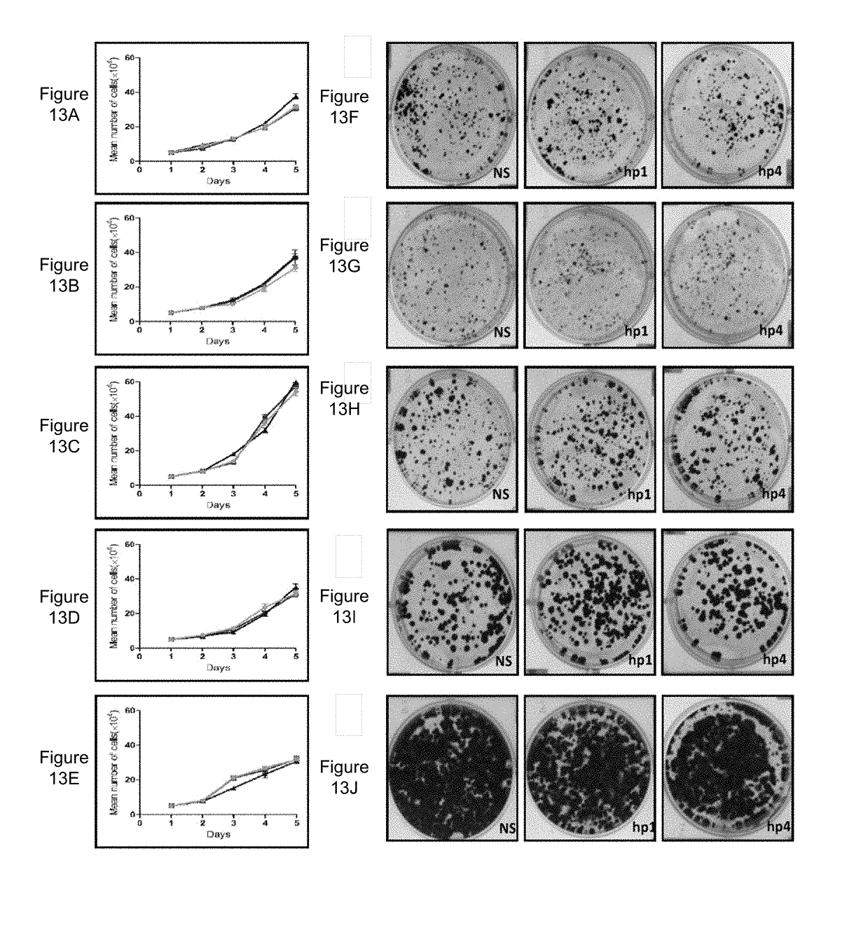

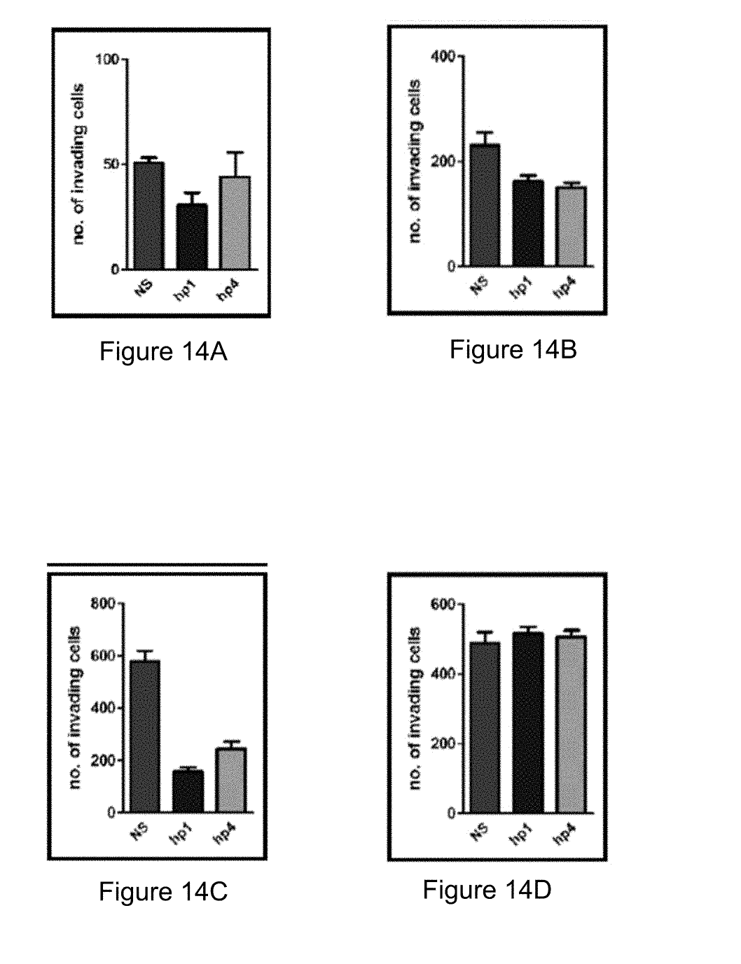

To further test this hypothesis, two complementary approaches were used to disrupt mTORC2 signaling and then cancer cell lines scored for changes in the frequency of asymmetrically dividing and G0-like cells. First, it was found that four structurally-different small molecules that inhibit both mTORC1 and mTORC2 signaling reduced the frequency of asymmetrically dividing and G0-like cells in both HCT116 and MCF7 breast cancer cells when used at low doses that did not appreciably inhibit cell proliferation (i.e., TORIN1, AZD8055, INK-128, Palomid-529) (FIG. 7E). In contrast, production of these cells was not suppressed either by two different inhibitors that preferentially target the TORC1 signaling complex alone (i.e., Rapamycin, RAD-001), or by a pan-class I PI3 kinase inhibitor (i.e., BKM-120), at target-suppressing doses (FIG. 7E). In addition, inducible shRNA knockdown of RICTOR (an obligate member of the mTORC2 signaling complex) with two different short hairpin RNAs suppressed the production of both asymmetrically dividing and slowly proliferating G0-like cells in a panel of five different human cancer cell lines, including those with a functional dependency on mutant PI3K (i.e., HCT116 (PIK3CAmutant), MCF7 (PIK3CAmutant), MDA-MB-231 breast, PC9 lung, and A375 melanoma) (FIGS. 7E, 1F, and 11C). However, RICTOR (−) cells did not differ from RICTOR (+) cells with respect to overall proliferation, response to stress (i.e., low serum, low glucose, or hypoxic conditions), or invasion in vitro. These results indicated that mTORC2 specifically induces asymmetric division and the production of slow proliferators, independent of PI3K or mTORC1 activity and without altering other important cancer cell functions (FIGS. 12A-12O, 12A-13J and 14A-14D).

Live-cell imaging experiments were performed to confirm mTORC2 regulation of asymmetric cancer cell division. Serial images of HCT116 and MCF7 cells dividing over seven days in culture either with or without RICTOR knockdown were obtained. These images were analyzed to identify individual dividing cells, lineage traces of these cells and their progeny created in order to identify sibling pairs, and differences plotted in mitotic times between sister cells arising from the same precursor. In the control population, eighty to ninety percent of dividing cells produced two siblings that divided again within five hours of each other (FIG. 7I, 7J). However, approximately ten to twenty percent of cells divided more asymmetrically to produce daughters with larger differences in time to mitosis. Remarkably, inducible shRNA knockdown of RICTOR reduced this minority fraction of the most asymmetrically dividing cells, thus decreasing inter-sibling asynchronicity and proliferative heterogeneity in the population. These findings confirmed that mTORC2 signaling induces asymmetric division in a small fraction of cancer cells to produce slow proliferators.

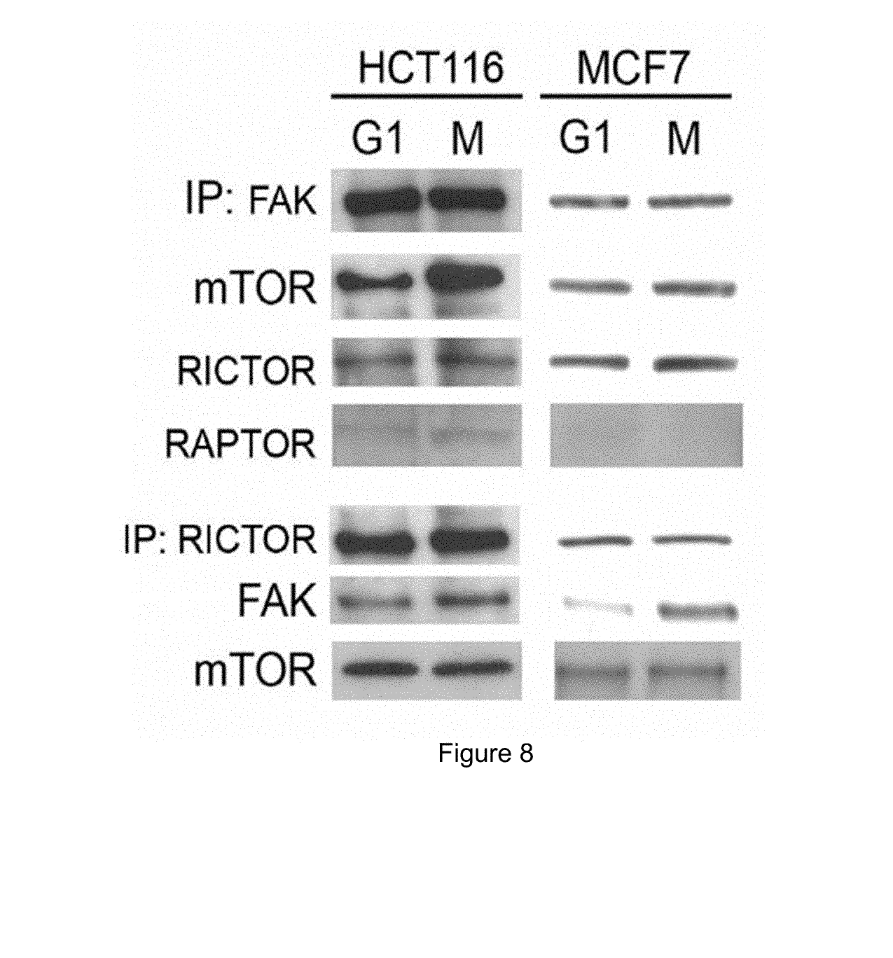

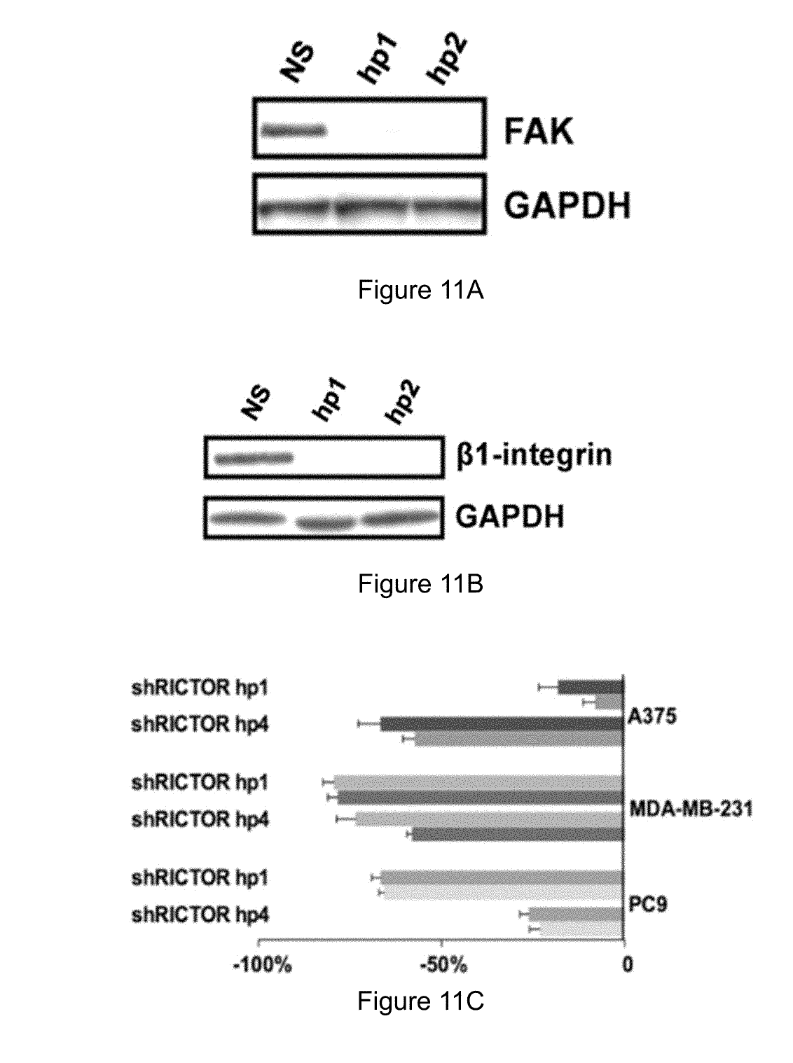

To identify proteins that physically interact with and might activate mTORC2 signaling during asymmetric division an immunoprecipitation (IP) approach was used. Specifically, it was found that IP with a RICTOR antibody (under conditions that maintain integrity of the mTORC2 complex in whole cell lysates) pulled down focal adhesion kinase (FAK) protein in both HCT116 and MCF7 (FIG. 8). Reciprocally, IP with a FAK antibody pulled down both mTOR kinase and RICTOR, confirming that FAK directly interacts with mTORC2 complex in these cells (FIG. 8). This observation indicated that FAK activity might somehow modulate mTORC2 signaling during asymmetric cancer cell division. Consistent with this hypothesis, it was found that inducible shRNA knockdown of FAK with two different short hairpins increased both asymmetrically dividing and G0-like cells in HCT116 and MCF7 (FIGS. 7F and 11A). Similarly, inhibiting FAK activity with two different small molecules (at doses that do not inhibit proliferation) increased the frequency of both asymmetrically dividing and G0-like cells (i.e., PF-562271, NVP-TAE226) (FIG. 7F). After RICTOR knockdown, however, FAK inhibitors failed to increase asymmetries or slow proliferators (FIG. 7H). These findings indicated that a loss of FAK activity enables mTORC2-mediated asymmetric cancer cell division.

Integrins are a family of heterodimeric transmembrane receptors that transduce signals from the extracellular matrix by activating a number of well-described signaling intermediaries within the cell, including FAK, to regulate cell cycle, shape, and motility in cancer and normal cells (9). It was reasoned that decreased integrin signaling might cause a loss of FAK activity resulting in mTORC2 activation during asymmetric division. In fact, inducible shRNA knockdown of β1-integrin (i.e., ITGB1, CD29) with two different short hairpins increased the fraction of asymmetrically dividing and G0-like cells in both HCT116 and MCF7 (FIGS. 7G and 11B). In addition, inhibiting β1-integrin function with two different monoclonal antibodies also increased both asymmetrically dividing and G0-like cells (i.e., A2B2, P4C10) (FIG. 7G) 910). In contrast, activating β1-integrin signaling with two other monoclonal antibodies, which force β1-integrin into an “on” state by imposing a conformational change, eliminated both asymmetries and slow proliferators in these cell lines (i.e., TS2/16, 12G10) (FIG. 7G) (10). These results indicated that an asymmetric loss in β1-integrin signaling during mitosis is both necessary and sufficient for asymmetric division and slow proliferators.

It was noted, however, that asymmetrically dividing cancer cells express β1-integrin protein uniformly on their cell membrane, suggesting that these cells might arise through the asymmetric β1-integrin activity. Since Type-I collagen is the major extracellular matrix protein that binds to and activates β1-integrin, it was hypothesized that polarities created by random variation in Type-I collagen might result in the asymmetric engagement of β1-integrin on dividing cancer cells in cell culture (11). To explore this hypothesis, MCF7 cells were grown on engineered matrices that display Type-I collagen fibrils aligned in a stereotypical pattern (data not shown). This maximized the probability that these cancer cells would divide in a relatively uniform collagen microenvironment, thus assuring the symmetrical activation of β1-integrin in all dividing cells within the population. Interestingly, MCF7 cells grown in this structured matrix did not produce asymmetries or G0-like cells unlike typical cell culture, indicating that a functional asymmetry in β1-integrin signaling related to irregularities in Type I collagen produces slow proliferators in vitro (FIG. 7H).

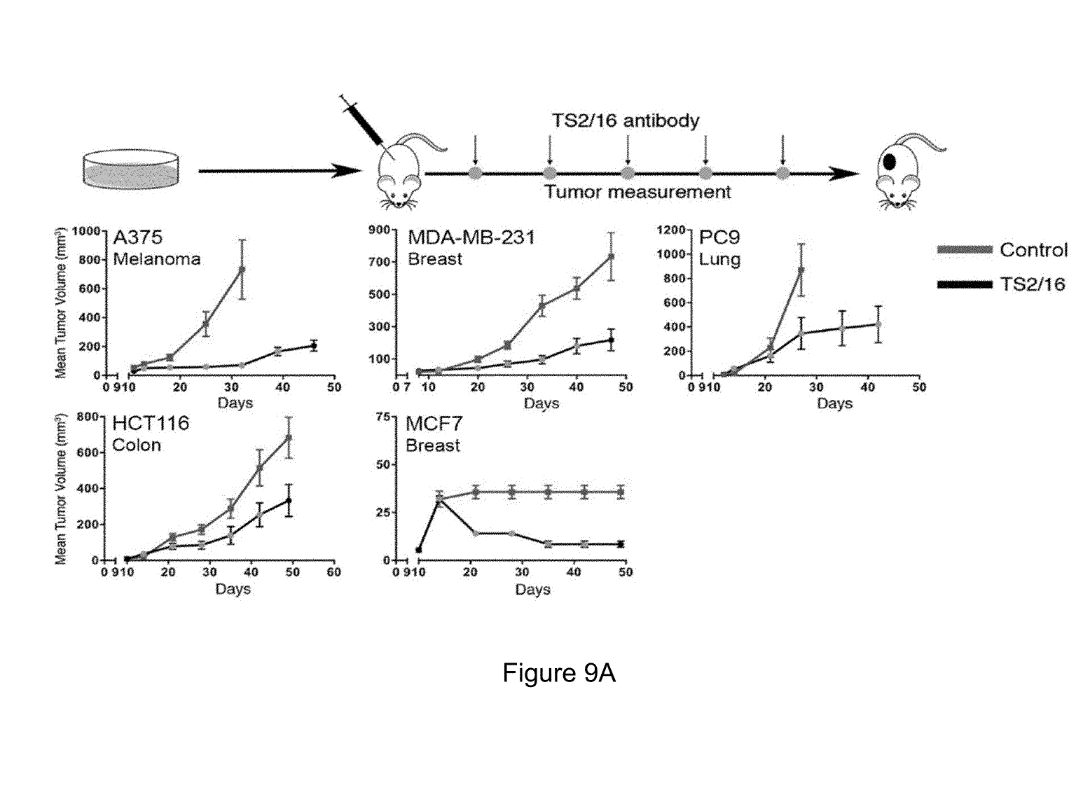

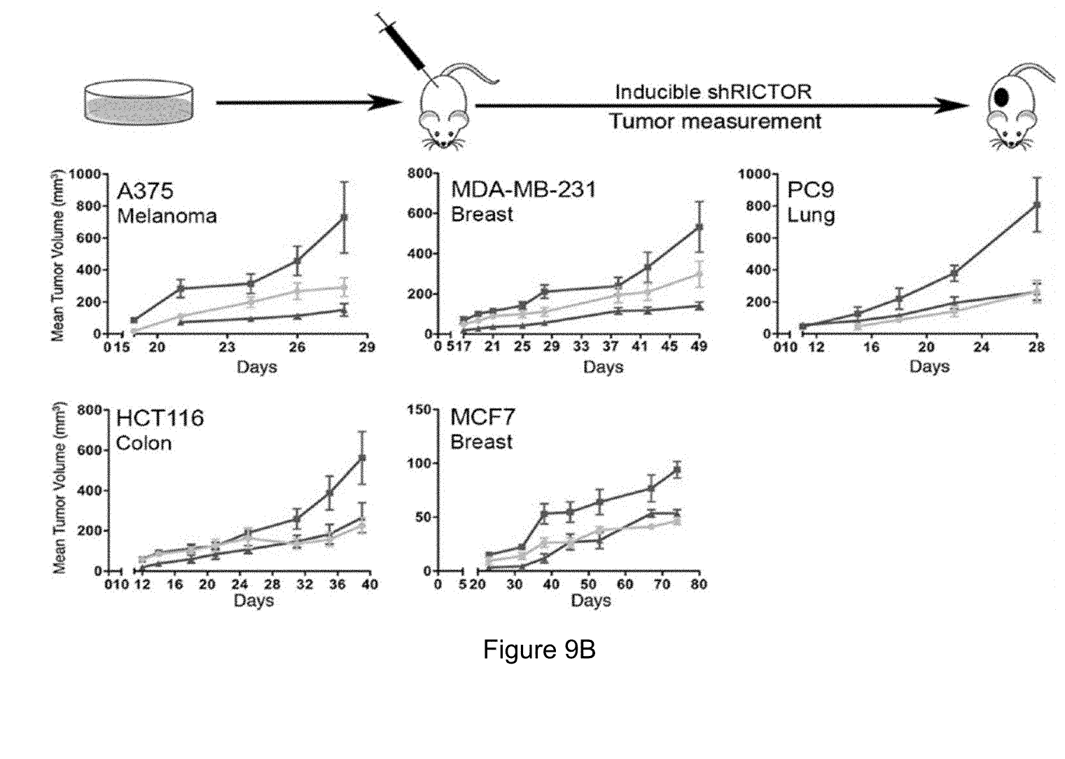

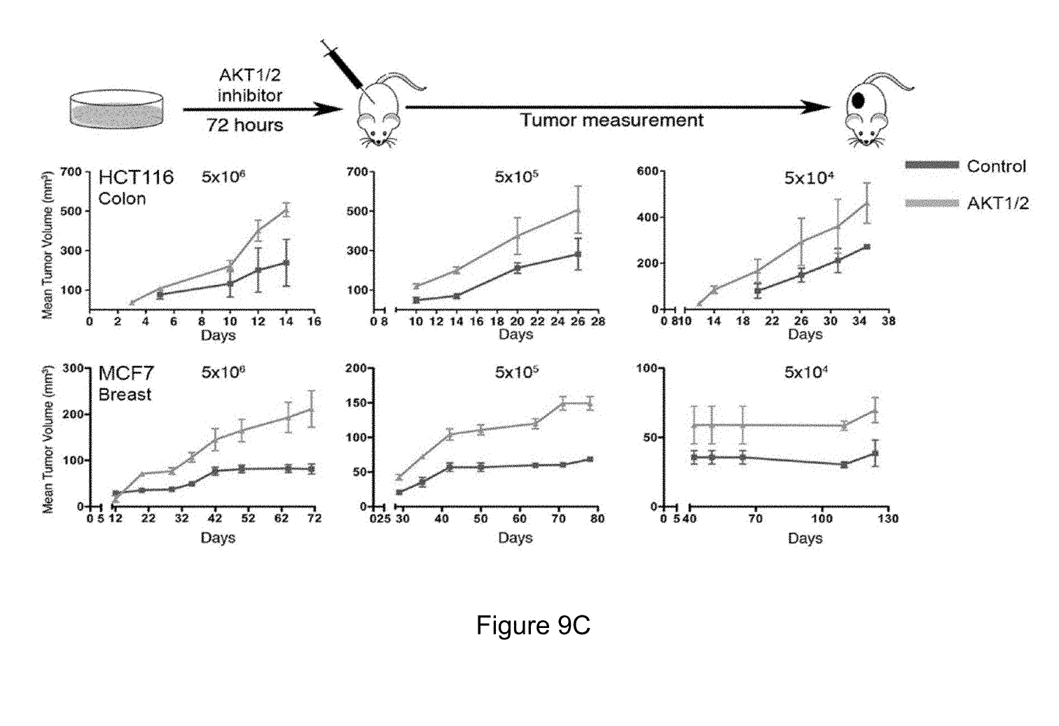

These results indicate that cancer cells in long-term culture retain a signaling pathway to produce slow proliferators despite the selective pressure for rapid and uniform proliferation, suggesting that this pathway might provide cancer cells with some fundamental advantage. To test this hypothesis, it was asked whether activating β1-integrin signaling in cancer cells (to prevent the production of slow proliferators) affects tumor growth in vivo. five cancer cell lines were transplanted subcutaneously into nude mice. When palpable tumors formed (i.e., A375, MDA-MB-231, PC9, HCT116, and MCF7), tumor-bearing animals were treated with the TS2/16 antibody while the growth of established tumors was followed (100 μl at 4 mg/ml/wk×5 weeks). Interestingly, TS2/16 treatment resulted in markedly slower tumor growth compared to control across this spectrum of solid tumor models, which included melanoma, lung, colorectal, and breast cancers (FIG. 9A). This finding was notable given that integrin signaling is generally thought to promote cancer cell proliferation, survival, and invasion (12). Since TS2/16 specifically activates human β1-integrin, moreover, these anti-tumor effects most likely resulted from the direct targeting human cancer cells rather than mouse stroma in these xenografts. In addition, it was asked whether disrupting mTORC2 signaling with RNA interference (which also reduces asymmetric division and slow proliferators without altering general cancer cell viability) would retard tumor growth (FIGS. 7E, 1F and 11C). The same cancer cell lines carrying doxycycline-inducible shRNAs for RICTOR were again injected subcutaneously in nude mice. These mice were fed doxycycline to induce continuous RICTOR knockdown in these cancer cells while tumor formation was monitored over time. Similar to β1-integrin activation, it was found that inhibiting mTORC2 signaling to reduce the production of slow proliferators also decreased growth of these different solid tumor types, (FIG. 9B). Both of these striking results supported the idea that tumors derive a considerable growth advantage from continuously producing slow proliferating cancer cells.

It was also asked whether cancer cell populations with an increased number of slow proliferators form tumors more efficiently in vivo. A low dose of the allosteric inhibitor AKT1/2 was used to partially inhibit AKT signaling in HCT116 and MCF7 cells, thus increasing their proportion of slowly proliferating AKT1low cells ex vivo (see FIG. 7C) (1). Then, these pre-treated cells were implanted subcutaneously in immune-compromised nude mice to assess tumor formation without further manipulation in vivo. Interestingly, this transient increase in G0-like cells significantly promoted the engraftment of these moderately tumorigenic cell lines across a 100-fold range, resulting in notably larger tumors over time (FIG. 9C). These findings demonstrate that AKT1low slow proliferators increase the tumorigenicity of cancer cell populations as xenotransplants.

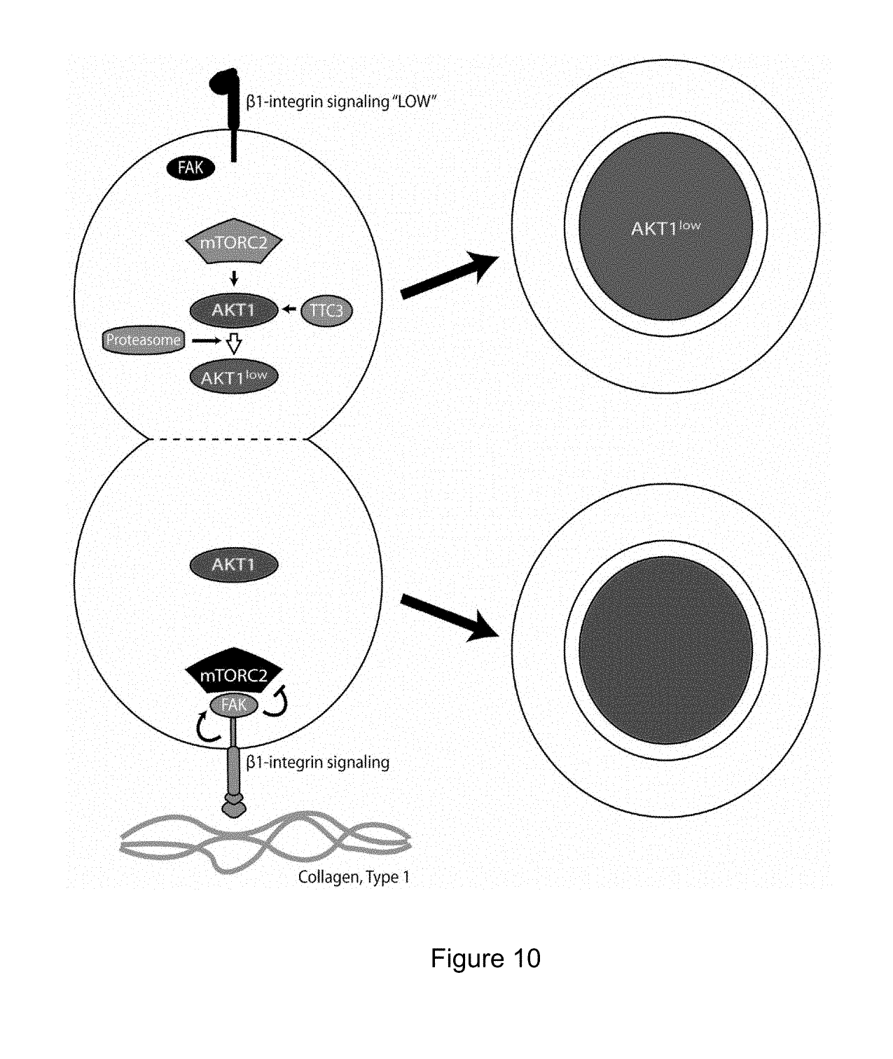

These findings demonstrate that a dividing cancer cell encountering an asymmetric decrease in β1-integrin/FAK signaling during mitosis activates mTORC2 signaling, which induces AKT1 degradation by TTC3 and the proteasome, to asymmetrically produce an AKT1low daughter cell (FIG. 10). This AKT1low cancer cell temporarily arrests its cell cycle, and superficially expresses a quiescent marker profile (i.e., MCM2low, H3K9me2low, HES1high), but can begin cycling again if its β1-integrin sensor is ligated optimally (1). Asymmetrically dividing cancer cells are not a fixed subpopulation, but rather appear to arise randomly depending on interaction with extracellular matrix proteins like collagen. Furthermore, slowly proliferating AKT1low cancer cells do not differentiate as far as we know. Nevertheless, AKT1low slow proliferators mimic cancer stem cell properties in being ROSlow, slow cycling, differentially tumorigenic in nude mice, and resistant to cytotoxic drugs (13,14). Importantly, it was found that selectively inhibiting production of these slow proliferators (either by activating β1-integrin with a monoclonal antibody or interfering with mTORC2 signaling) impedes the growth of biologically diverse solid tumor types in vivo. It was also noted that integrin receptors mark epithelial stem cells and dictate their self-renewal in normal tissues (11,15,16). Without wishing to be limited by theory, these observations suggest that a fundamental signaling pathway required for malignant stem cell homeostasis has been identified herein. It is speculated that rapidly dividing cancer cells facultatively trigger this stem-like pathway to spawn slow proliferators when they encounter disorganized extracellular matrix associated with a suboptimal tumor microenvironment (17). In turn, slow proliferators may buffer proliferating cancer cell populations against this cytotoxic stress within growing tumors, adding to tumor bulk as they accumulate over time, to proliferate as local conditions permit. This model of tumor dynamics complements clonal selection theory and may provide deeper insight into tumor progression, dormancy, and treatment resistance (18).

### REFERENCES

1. I. Dey-Guha et al., Asymmetric cancer cell division regulated by AKT. Proceedings of the

National Academy of Sciences of the United States of America 108, 12845 (Aug. 2, 2011).
2. J. A. Magee, E. Piskounova, S. J. Morrison, Cancer stem cells: impact, heterogeneity, and uncertainty. Cancer cell 21, 283 (Mar. 20, 2012).
3. K. Ericson et al., Genetic inactivation of AKT1, AKT2, and PDPK1 in human colorectal cancer cells clarifies their roles in tumor growth regulation. Proceedings of the National Academy of Sciences of the United States of America 107, 2598 (Feb. 9, 2010).
4. T. Okuzumi et al., Inhibitor hijacking of Akt activation. Nature chemical biology 5, 484 (July, 2009).
5. H. Jo et al., Small molecule-induced cytosolic activation of protein kinase Akt rescues ischemia-elicited neuronal death. Proceedings of the National Academy of Sciences of the United States of America 109, 10581 (Jun. 26, 2012).
6. F. Suizu et al., The E3 ligase TTC3 facilitates ubiquitination and degradation of phosphorylated Akt. Developmental cell 17, 800 (December, 2009).
7. D. D. Sarbassov, D. A. Guertin, S. M. Ali, D. M. Sabatini, Phosphorylation and regulation of Akt/PKB by the rictor-mTOR complex. Science 307, 1098 (Feb. 18, 2005).
8. B. D. Manning, L. C. Cantley, AKT/PKB signaling: navigating downstream. Cell 129, 1261 (Jun. 29, 2007).
9. R. 0. Hynes, Integrins: bidirectional, allosteric signaling machines. Cell 110, 673 (Sep. 20, 2002).
10. A. Byron et al., Anti-integrin monoclonal antibodies. Journal of cell science 122, 4009 (Nov. 15, 2009).
11. R. O. Hynes, The extracellular matrix: not just pretty fibrils. Science 326, 1216 (Nov. 27, 2009).
12. J. S. Desgrosellier, D. A. Cheresh, Integrins in cancer: biological implications and therapeutic opportunities. Nature reviews. Cancer 10, 9 (January, 2010).
13. M. Al-Hajj, M. S. Wicha, A. Benito-Hernandez, S. J. Morrison, M. F. Clarke, Prospective identification of tumorigenic breast cancer cells. Proceedings of the National Academy of Sciences of the United States of America 100, 3983 (Apr. 1, 2003).
14. M. Diehn et al., Association of reactive oxygen species levels and radioresistance in cancer stem cells. Nature 458, 780 (Apr. 9, 2009).
15. F. M. Watt, B. L. Hogan, Out of Eden: stem cells and their niches. Science 287, 1427 (Feb. 25, 2000).
16. S. Goulas, R. Conder, J. A. Knoblich, The Par complex and integrin s direct asymmetric cell division in adult intestinal stem cells. Cell stem cell 11, 529 (Oct. 5, 2012).
17. E. Brown et al., Dynamic imaging of collagen and its modulation in tumors in vivo using second-harmonic generation. Nature medicine 9, 796 (June, 2003).
18. P. C. Nowell, The clonal evolution of tumor cell populations. Science 194, 23 (Oct. 1, 1976).

### Materials & Methods

Cell culture. HCT116 colon, MCF7 breast, MDA-MB-231 breast, A375 melanoma, and PC9 lung cancer cells were purchased from the American Type Culture Collection (ATCC). HCT116 AKT1/2−/− cells we purchased from Horizon Discovery (Cambridge, UK). MCF7 and MDA-MB-231 cells were maintained in DMEM, 10% FCS, 40mM glutamine, 100 U/mL penicillin, and 100 μg/mL streptomycin. HCT116 and HCT116 AKT1/2−/− cells were maintained in McCoy's 5a medium supplemented with 10% FCS, 100 U/mL penicillin, and 100 μg/mL streptomycin. PC9 cells were maintained in RPMI, 25% glucose, 1% sodium pyruvate, 100 U/mL penicillin, and 100 μg/mL streptomycin. A375 cells were maintained in DMEM supplemented with high glucose HEPES buffer, 10% FCS, 100 U/mL penicillin, and 100 μg/mL streptomycin. All the cells were grown in a humidified atmosphere at 37° C. and 5% CO2.

Drug treatment in vitro. Cells were seeded onto collagen IV-coated coverslips, allowed to attach overnight, and treated with vehicle (DMSO) or AKT1/2 inhibitor (HCT116: 20 μM; MCF7: 2 μM) (Sigma), MK2206 (HCT116: 10 μM; MCF7: 3 μM) (Selleck Chemicals), TORIN1 (HCT116: 0.5 μM; MCF7: 0.25 μM) (Tocris Bioscience), AZD8055 (HCT116: 0.7 μM; MCF7: 0.1 μM) (Selleck Chemicals), INK128 (HCT116: 0.05 μM; MCF7: 0.01 μM) (Active Biochem), Palomid 529 (HCT116: 10 μM; MCF7: 20 μM) (Selleck Chemicals), (Rapamycin (HCT116: 20 μM; MCF7: 20 μM)(Sigma), RAD-001 (HCT116: 10 μM; MCF7: 5 μM) (Selleck Chemicals), BKM-120 (HCT116: 1.5 μM; MCF7: 0.5 μM) (Active Biochem), FAK inhibitors (PF-562271: 1 μM Pfizer) and NVP-TAE226 : 1 μM (Novartis), for both cell lines) for 72 h and Bortezemib (HCT116: 1 μM; MCF7: 4 μM) (Selleck Chemicals) MG-132 (vehicle: ethanol) (HCT116: 1 μM; MCF7: 10 μM), for 24 h.

shRNA constructs. Human TRIPZ lentiviral inducible shRNAmirs for RICTOR (Clone ID: V2THS_120392, V2THS_120389, V2THS_38014, V2THS_225915), FAK (Clone ID: V2THS_57326, V2THS_325805), β1-integrin (Clone ID: V2THS_133469, V2THS_390997), non-silencing, and empty vector were purchased from Open Biosystems and virus was generated using a standard protocol. Infection was performed 24 h later in MCF7, HCT116, A375, PC9 and MDA-MB-231 cell lines with the lentiviral particles followed by selection with 2 μM puromycin. Following selection, cells were allowed to grow to confluency. The shRNAs were induced using 2 μg/ml doxycycline for 72 h. The TTC3 virus was purchased from Sigma-Aldrich and infected in HCT116 and MCF7 cells and the standard protocol for selection was followed.

Generation of AKT1 mutant cell lines. AKT1(WT) cDNA was purified using PCR after cutting PDD AKT1(WT) with restriction enzymes BamHI and Xhol. Following purification, the product was ligated into pMSCVpuro-C-tag-mCherry cut with BglII and SalI. All the AKT1mutants were generated using the QuikChange site directed mutagenesis kit (Agilent technologies) and the product was ligated into pMSCVpuro-C-tag-mCherry. The resulting vector pMSCV-puro-AKT1-mCherry was sub-cloned into DH5α competent cells (Invitrogen). Sequencing verification of the fusion product was performed by the MGH DNA Core Facility with primers pMSCV 5′-CCCTTGAACCTCCTCGTTCGACC-3′ (SEQ ID NO: 1) and pMSCV 3′-GAGACGTGCTACTTCCATTTGTC-5′ (SEQ ID NO: 2). Virus carrying the desired fusion gene was produced by transfecting 293-T cells with target vector pMSCV-puro- AKT1-mCherry and packaging vector pCL-Ampho using the Mirus Tran1T-293 transfection reagent and established protocols. Virus was collected 24 h following transfection. Before infection, cells were plated in a six-well plate in DMEM, 10% FCS. Infection was performed 24 h later by adding 0.5 mL DMEM, 10% FCS, 0.5mL pooled virus, and 1 μL 1,000× polybrene per well. A media change was performed the following day and cells were allowed to grow to confluency before splitting into a 10-cm dish and selection with 2 puromycin. Following selection, cells were allowed to grow to confluency before clones were selected using single-cell sorting (Becton Dickinson FACSAria II™). Single cells were filtered by gating on the brightest 5% of cells in the PETexas red channel and sorted into individual wells of a 96-well plate. Clones were harvested between 14 and 21 days.

Immunofluorescence staining. For bulk populations and for colonies, cells were grown directly on collagen IV-coated coverslips (Sigma). Cells were fixed in 3.7% formalin, permeabilized using 0.1% Triton X-100, and treated with 0.1% SDS. They were blocked in 1% BSA and then incubated with primary antibody (a-H3K9me2 (Abcam); α-MCM2 and α-Tubulin (Cell Signaling), α-Hes1 and α-TTC3 (Abnova)) diluted in blocking solution, washed, and incubated with the respective secondary antibody. Cells were mounted using hard-set mounting media containing DAPI (Vector Laboratories). All secondary antibodies were Alexa Fluor conjugates (488, 555, 568, 633, and 647) (Invitrogen). Immunofluorescence imaging (on a Nikon Eclipse Ti A1R-A1™ confocal microscope) and live-cell imaging (on the Nikon Biostation CT platform) were performed as previously described7. Slides coated with Type-I collagen (control) and AlignCol™ woven with large collagen fibers (100-200 nm) (Advanced Biomatrix) were incubated for 1 hour in 70% ethanol and then washed with PBS. Cells were then plated on the slides and processed for immunofluorescence.

Immunoprecipitation. Cells were rinsed with PBS, fixed with 0.37% formaldehyde and quenched with 0.25M glycine. Cell lysates were prepared in lysis buffer (1% Triton X-100, 150 mM NaCl, 3mM MgCl, 40 mM HEPES [pH 7.5], 50 mM NaF, EDTA-Free protease inhibitor and phosphatase inhibitor [Roche]) and then incubated with rabbit serum as control or primary antibody (α-FAK (AbCam), (α-RICTOR (Santa Cruz), for 4 hours followed by 50% slurry of protein G-sepharose (Roche) for 1 hour. The immunoprecipitates were washed and resolved by SDS-PAGE. For western blots: primary antibody: RICTOR, mTOR (Cell Signaling), Raptor, FAK, β1-integrin, TTC3 (AbCam).

Antibody activation and inhibition. Cells were incubated in media containing 10% FBS and the antibody:inhibiting (A2B2:20 μg/ml (Developmental Studies Hybridoma Bank), (P4C10: 10 μg/ml (Millipore) and activating (TS2/16 and 12G10: 10 μg/ml) (Santacruz), for 1 hour at 4° C. Cells were then plated on collagen IV-coated coverslips (Sigma) and incubated in the antibody at 37° C., for 24 hours.

Generation of mCerulian-tagged cell lines. Virus carrying the pMSCV-CMV-NLSmCerulean construct was produced by transfecting 293-T cells plated at 500,000 cells per well in a six-well plate. Twenty four hours later, these cells were transfected with lag target vector pMSCV-CMVNLS-mCerulean, lag packaging vector pCL-Ampho, and 34 FuGENE HD mixed with 100 μL reduced serum solution (Opti-MEM; Invitrogen). Virus was collected 24 h following transfection. Before infection with virus, HCT116 or MCF7 cells were plated at 50,000 cells per well in a six-well plate in DMEM, 10% FCS. Infection was performed 24 h later by adding 0.5 mL DMEM, 10% FCS, 0.5 mL pooled virus, and 1 μL 1,000× polybrene per well. A media change was performed the following day, and cells were allowed to grow to confluency before splitting into a 10-cm plate. HCT116 or MCF7/NLS-mCerulean cells were selected using fluorescence-activated cell sorting (Becton Dickinson FACSAria II™) and gating on the brightest 5% cells in the Pacific blue channel

Live-cell imaging & time-lapse analysis. In order to follow the fate of HCT116 cells in vitro, we plated HCT116 cells tagged with NLS-mCerulean and also a doxycycline-inducible non-silencing or Rictor knockdown shRNA (hp4) construct in glass-bottom 12-well plates (MatTek Product # P12G-1.0-10-F) treated with type IV collagen. Tagged HCT116 cells were plated in 2 μg/ml of doxycycline at a density of 1000 cells per well along with unlabeled HCT116 cells at a density of 4000 cells per well. All cells were initially grown in McCoy's 5-alpha+10% FCS at t=0. We performed media changes every day with 2 μg/ml doxycycline. Multi-point serial imaging was performed using an inverted microscope fitted with a tissue culture incubator (Nikon Ti-Eclipse) every 20 minutes at 20× magnification (CFI Plan Apo 20×) for 164 hours. Both phase and fluorescent images were captured. Cells were excited with an LED (Nikon C-HGFI Intensilight HG Illum™) and passed through a filter series (Nikon, C-FL CFP and RFP HC HISN Zero Shift Filter Set). All cell division events were tracked manually using the CFP images by recording the following characteristics for each cell: ID based on initial frame of appearance and x/y coordinate, first frame, last frame, origin ID, progenitor IDs, and x/y coordinates for first and last frame, and end method (division, lost in tracking, lost to wash out, or lost to cell death). Each point is calculated at 20-minute intervals and only shown if there was at least one event occurring. Analysis was performed using R v2.8.0 (The R Foundation for Statistical Computing, 2008) by analyzing all division events.

Proliferation assays. HCT116 colon, MCF7 breast, MDA-MB-231 breast, A375 melanoma, and PC9 lung cancer cells carrying either doxycycline-inducible non-silencing or RICTOR-targeting shRNAs (120392, 225915) were plated in a 12- well plate at a density of 50,000 cells/per well in triplicate with doxycycline containing medium on day 1 and the cells were counted every 24 hrs for 5 days. Doxycycline containing medium was replaced everyday. Cells were maintained at: 1) 21% oxygen, 10% fetal calf serum and 25 mM D-glucose (normal condition), 2) 4% oxygen (hypoxia), 3) 1% serum (low serum), or 4) 5.56 mM D-glucose (low glucose).

Clonogenic assays. HCT116 colon, MCF7 breast, MDA-MB-231 breast, A375 melanoma, and PC9 lung cancer cells carrying either doxycycline-inducible non-silencing or RICTOR-targeting shRNAs (120392, 225915) were plated in a 6- well plate at a density of 1,000 cells/per well in triplicate with doxycycline containing medium on day 1. Cells were allowed to grow into small colonies for 5 days and then irradiated at a dose of 2Gy. Colonies were then allowed to grow for another 2 weeks and were stained using 0.125% Coomasie Blue. Doxycycline containing medium was replaced everyday.

Invasion assays. HCT116 colon, MCF7 breast, MDA-MB-231 breast, A375 melanoma, and PC9 lung cancer cells carrying either doxycycline-inducible non-silencing or RICTOR-targeting shRNAs (120392, 225915) were induced with Doxycycline (2 μg/ml) for 72 hrs and then seeded onto Matrigel invasion chambers at a density of 50,000 cells per well in triplicate. Doxycycline containing medium was replaced everyday. The invasion chambers were incubated for 24 hrs at 37° C. and 5% CO2. The chamber filters were then stained using 0.125% Coomasie Blue and mounted onto glass slides.

Tumor studies in vivo. For TS2/16 antibody treatment studies in vivo, we injected 5×105 cells (A375, MDA-MB-231, PC9, HCT116, MCF7) subcutaneously into the flanks of 5-6 week old, female nude mice (Nude/Nude) (Charles River Labs). Once the tumors were palpable, mice were injected i.p. with TS2/16 (1000 at 4 mg/ml/wk×5 weeks) and the tumors were measured. For RICTOR knockdown experiments in vivo, we injected 5×105 cells (A375, MDA-MB-231, PC9, HCT116, MCF7) carrying either doxycycline-inducible non-silencing or RICTOR-targeting shRNAs (120392, 225915) subcutaneously into the flanks of 5-6 week old, female nude mice (Nude/Nude). The mice were given doxycycline in water at 20 mg/ml for hairpin induction starting immediately after implantation. For induction of slow proliferators, cells were treated with AKT1/2 inhibitor and DMSO (vehicle) for 72 h and were harvested at 60-70% confluence, and then counted and washed twice in PBS and resuspended in 1:1 Media: Matrigel (BD Biosciences). We then injected 5×106, 5×105, and 5×104 cells respectively subcutaneously into the flanks of 5-6 week old, female nude mice. For all experiments, growing tumors were measured weekly by caliper, and mice were killed when tumors reached approximately 1 cm3 in size. Animal experiments were carried out under a Massachusetts General Hospital Institutional Review Board-approved protocol.

### Example 4

All cancers contain an admixture of rapidly and slowly proliferating cancer cells. This proliferative heterogeneity complicates the diagnosis and treatment of patients with cancer because slow proliferators are hard to eradicate, can be difficult to detect, and may cause disease relapse sometimes years after apparently curative treatment. While clonal selection theory explains the presence and evolution of rapid proliferators within cancer cell populations, the circumstances and molecular details of how slow proliferators are produced is not well understood.

Described herein is the discovery of a β1-integrin/FAK/mTORC2/AKT1-associated signaling pathway that can be triggered for rapidly proliferating cancer cells to undergo asymmetric cell division and produce slowly proliferating AKT1^{low} daughter cells. In addition, evidence indicates that the proliferative output of this signaling cascade involves a proteasome-dependent degradation process mediated by the E3 ubiquitin ligase TTC3. These findings reveal that proliferative heterogeneity within cancer cell populations, in part, is produced through a targetable signaling mechanism, with implications for understanding cancer progression, dormancy, and therapeutic resistance.

These findings provide a deeper understanding of the proliferative heterogeneity that exists in the tumor environment and highlight the importance of therapies against multiple proliferative contexts.

Introduction

In cell culture, dividing cancer cells usually produce two daughter cells that divide again in relative synchrony within a few hours of each other. Occasionally, however, a cancer cell divides to produce progeny that are asynchronous with respect to the next cell cycle, with one daughter cell having a markedly slower cell division time than the other, on the order of days. As described herein, this proliferative heterogeneity correlates with cancer cells asymmetrically suppressing AKT protein kinase levels by about ninety percent during mitosis just before cytokinesis (1). These rare asymmetries produce one AKT^{normal} daughter cell that rapidly enters the next cell cycle and another AKT^{low} cell that remains dormant for a more prolonged time before dividing again. Slowly cycling AKT^{low} cells reduce their production of reactive oxygen species (i.e., ROS^{low} ), downregulate proliferation proteins (e.g., MKI67^{low}, MCM2^{low}), suppress multiple nuclear histone marks similar to quiescent cell populations (e.g., H3K9me2^{low}), and transcriptionally upregulate the HES1 transcription factor that may mark exit from the cell cycle into G0 (i.e., HES1high; ref 1). As AKT^{low} cells do eventually divide, converting to a AKT^{normal} proliferative phenotype overtime, these cells are referred to herein as “G0-like” to describe this temporary and reversible cell state. Significantly, as described herein, AKT^{low} cancer cells within actual human breast tumors are highly resistant to prolonged treatment with combination chemotherapy using adriamycin, cyclophosphamide, and paclitaxel, indicating these slow proliferators constitute an important but unappreciated reservoir of treatment resistance in patients with breast cancer. Understanding more precisely how AKT^{low} cancer cells arise at a molecular level will provide fundamental insight into cancer biology with clinical relevance.

Materials and Methods

Cell culture. HCT116 colon and MCF7 breast were purchased from the ATCC where they were authenticated. HCT116-AKT1/2^{-/-} cells were purchased from Horizon Discovery where they were authenticated. MCF7 cells were maintained in DMEM, 10% FCS, 40 mmol/L glutamine, 100 U/mL penicillin, and 100 μg/mL streptomycin. HCT116 and HCT116-AKT1/2^{-/-} cells were maintained in McCoy 5α medium supplemented with 10% FCS, 100 U/mL penicillin, and 100 μg/mL streptomycin. Cells were grown in a humidified atmosphere at 37° C. and 5% CO2.

Generation of AKT1-mutant cell lines. pDD AKT1(WT) and pMSCV-puro-Ctag-mCherry were utilized. AKT1(WT) cDNA was purified using PCR after cutting PDD AKT1(WT) with restriction enzymes BamHI and Xhol. After purification, the product was ligated into pMSCVpuro-C-tag-mCherry cut with BglII and SalI. All the AKT1 mutants were generated using the QuikChange Site Directed Mutagenesis Kit™ (Agilent Technologies) and the product was ligated into pMSCVpuro-C-tag-mCherry. The resulting vector pMSCV-puro-AKT1-mCherry was subcloned into DH5α-competent cells (Invitrogen). Sequencing verification of the fusion product was performed by the MGH DNA Core Facility with primers pMSCV 5′-CCCTTGAACCTCCTCGTTCGACC-3′ (SEQ ID NO: 1) and pMSCV 3′-GAGACG-TGCTACTTCCATTTGTC-5′ (SEQ ID NO: 2). Virus carrying the desired fusion gene was produced by transfecting HEK 293T cells with target vector pMSCV-puro-AKT1-mCherry and packaging vector pCL-Ampho using the Mirus TransIT-293™ transfection reagent and established protocols. Virus was collected 24 hours after transfection. Before infection, cells were plated in a 6-well plate in DMEM, 10% FCS. Infection was performed 24 hours later by adding 0.5 mL DMEM, 10% FCS, 0.5 mL pooled virus, and 1 μL 1,000× polybrene per well. A media change was performed the following day and cells were allowed to grow to confluency before splitting into a 10 cm dish and selection with 2 μmol/L puromycin. After selection, cells were allowed to grow to confluency before clones were selected using single-cell sorting (Becton Dickinson FACSAria II™). Single cells were filtered by gating on the brightest 5% of cells in the PE Texas red channel and sorted into individual wells of a 96-well plate. Clones were harvested between 14 and 21 days.

Drug treatment in vitro. Cells were seeded onto collagen IV-coated coverslips, allowed to attach overnight, and treated with vehicle (DMSO) or AKT1/2 inhibitor (HCT116:20 μmol/L; MCF7: 2 μmol/L; Sigma), MK2206 (HCT116: 10 μmol/L; MCF7: 3 μmol/L; Selleck Chemicals), TORIN1 (HCT116: 0.5 μmol/L; MCF7: 0.25 μmol/L; Tocris Bio-science), AZD8055 (HCT116: 0.7 μmol/L; MCF7: 0.1 μmol/L; Selleck Chemicals), INK128 (HCT116: 0.05 μmol/L; MCF7: 0.01 μmol/L; Active Biochem), Palomid 529 (HCT116: 10 μmol/L; MCF7: 20 μmol/L; Selleck Chemicals), Rapamycin (HCT116: 20 μmol/L; MCF7: 20 μmol/L; Sigma), RAD-001 (HCT116: 10 μmol/L; MCF7: 5 μmol/L; Selleck Chemicals), BKM-120 (HCT116: 1.5 μmol/L; MCF7: 0.5 μmol/L; Active Bio-chem), FAK inhibitors [PF-562271: 1 μmol/L (Pfizer) and NVP-TAE226 : 1 μmol/L (Novartis), for both cell lines] for 72 hours or 144 hours and bortezemib (HCT116: 1 μmol/L; MCF7: 4 μmol/L; Selleck Chemicals) MG-132 (vehicle: ethanol; HCT116: 1 μmol/L; MCF7: 10 μmol/L), for 24 hours.

shRNA constructs. Human TRIPZ lentiviral inducible shRNAmirs for RICTOR (clone ID: V2THS_120392, V2THS_120389, V2THS_38014, V2THS_225915), FAK (clone ID: V2THS_57326, V2THSβ1325805), β1-integrin (clone ID: V2THS_133469, V2THS_390997), nonsilencing, and empty vector were purchased from Open Biosystems and virus was generated using a standard protocol. Infection was performed 24 hours later in MCF7 and HCT116 cells with the lentiviral particles followed by selection with 2 μmol/Lpuromycin. After selection, cells were allowed to grow to confluency. The shRNAs were induced using 2 μg/mL doxycycline for 72 hours. The TTC3 virus was purchased from Sigma-Aldrich and infected in HCT116 and MCF7 cells and the standard protocol for selection was followed.

Antibody activation and inhibition. Cells were incubated in media containing 10% FBS and the respective β1-integrin antibody: inhibiting [AIIB2: 20 μg/mL (Developmental Studies Hybridoma Bank), P4C10: 10 μg/mL (Millipore)], and activating (TS2/16 and 12G10: 10 μg/mL; Santa Cruz Biotechnology), for 1 hour at 4° C. Cells were then plated on collagen IV-coated coverslips (Sigma) and incubated in the antibody at 37° C. for 24 hours.

Immunofluorescence staining. Cells were grown directly on collagen IV-coated coverslips (Sigma). Cells were fixed in 3.7% formalin, permeabilized using 0.1% Triton X-100, and treated with 0.1% SDS. They were blocked in 1% BSA and then incubated with primary antibody (α-H3K9me2, α-Hes1 α-TTC3, and α-AKT(phospho-T308; Abcam); α-MCM2, α-Tubulin, α-pan-AKT, and α-AKT(phos-pho-5473; Cell Signaling Technology), diluted in blocking solution, washed, and incubated with the respective secondary antibody. Cells were mounted using hardset mounting media containing DAPI (Vector Laboratories). All secondary antibodies were Alexa Fluor conjugates (488, 555, 568, 633, and 647; Invitrogen).

Collagen matrix studies. Slides coated with type-I collagen (control) and AlignCol™ woven with large collagen fibers (100-200 nm; Advanced Bio-matrix) were incubated for 1 hour in 70% ethanol and then washed with PBS. Cells were then plated on the slides, incubated for 24 hours, and processed for immunofluorescence.

Confocal imaging. Immunofluorescence imaging was performed on a Nikon Eclipse Ti A1R-A1™ confocal microscope. G0-like slow proliferators were specifically identified as cells in the bottom 10% of coincident staining for MCM2, H3K9me2, and HES1. Cells were scored by counting G0-like versus other proliferating cancer cells among 10,000 cells from multiple fields of view at 20× magnification.

Western blotting and immunoprecipitation. Standard protocols were used for SDS-PAGE electrophoresis and the following primary antibodies: α-RICTOR, α-mTOR, Phospho-AKT-Ser473 (D9E; Cell Signaling Technology) and α-RAPTOR, α-FAK, α-β1-integrin, α-TTC3, Pan-AKT, and GAPDH (Abcam). For immunoprecipitation studies, cells were synchronized with 200 ng/mL of nocodazole for 12 hours and then released for 2 hours. Cells were rinsed with PBS, fixed with 0.37% formalde-hyde, and quenched with 0.25 mol/L glycine. Cell lysates were prepared in lysis buffer [1% Triton X-100, 150 mmol/L NaCl, 3 mmol/L MgCl, 40 mmol/L HEPES (pH 7.5), 50 mmol/L NaF, EDTA-free protease inhibitor and phosphatase inhibitor (Roche)] and centrifuged at 14,000×g for 10 minutes. Supernatant (250 μg) was incubated with the indicated antibodies [α-FAK (AbCam), α-RICTOR (Santa Cruz Biotechnology)], for 4 hours at 4° C. with rotation and then with 50 μL of a 50% slurry of protein G-sepharose (Roche) for 1 hour. Immunoprecipitates were washed and resolved by SDS-PAGE electrophoresis.

Results

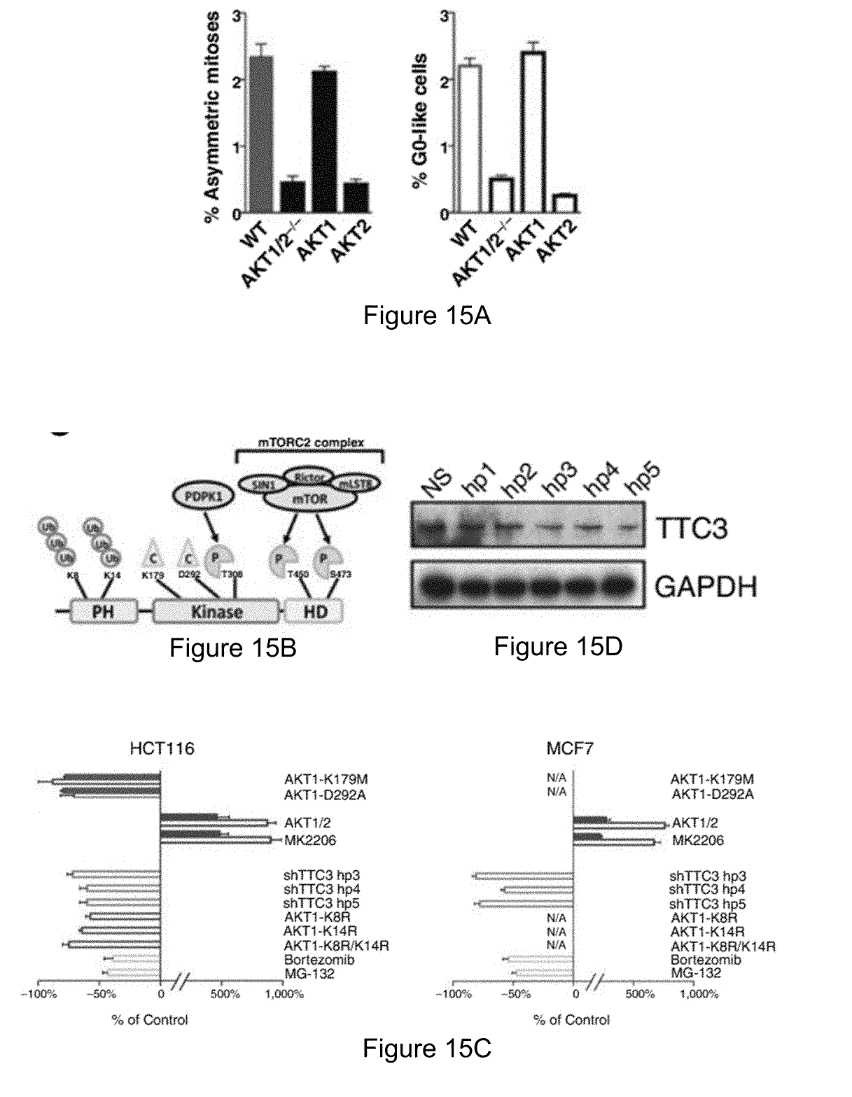

As AKTlow cancer cells only partially suppress total AKT protein levels, it was first asked whether asymmetric division occurs in the complete absence of all three AKT isoforms (i.e., AKT1, AKT2, and AKT3). To do so, HCT116 colorectal cancer cells with adeno-associated virus (AAV)-mediated disruption of the AKT1 and AKT2 gene loci (i.e., AKT1/2−/− cells; ref 2) were obtained. Importantly, AKT1/2−/− cells do not express either AKT1 or AKT2, nor do they express AKT3, and thus survive and proliferate in the complete absence of AKT signaling, presumably through compensatory changes that arose during their initial selection. Confocal microscopy was used to score AKT1/2−/− cell populations for rare, asymmetrically dividing, and G0-like cancer cells that express the previously validated H3K9me2low/MCM2 low/HES1high molecular profile, which specifically marks AKTlow slow proliferators previously shown (data not shown; ref. 1). Interestingly, this AKT1/2−/− line had virtually no asymmetrically dividing or G0-like cells compared with wild-type HCT116 (the parental line from which AKT1/2−/− cells are derived; FIG. 15A). However, lentiviral-mediated overexpression of an AKT1 cDNA in AKT1/2−/− cells completely restored formation of both asymmetrically dividing and G0-like cells, while overexpression of AKT2 did not, indicating that AKT1 is both necessary and sufficient for the production of G0-like cells (FIG. 15A).

On the basis of this result, site-directed mutagenesis was used to identify AKT1 domains that might be required for its partial suppression during asymmetric division. A series of AKT1 cDNA constructs with mutations in critical amino acids known to be important for various aspects of AKT1 signaling were created (FIG. 15B). Each mutant AKT1 construct was then overexpressed in AKT1/2−/− cells and these engineered cells scored for both asymmetrically dividing and G0-like cancer cells. It was first asked whether AKT1 kinase activity was necessary for production of these slow proliferators. It was found that AKT1-K179M (a commonly studied mutation in the kinase pocket that renders AKT1 catalytically dead) failed to restore production of asymmetrically dividing and G0-like cells in the AKT1/2−/− line (FIG. 15A). In addition, AKT1-D292A (a mutant hypomorph with diminished kinase catalytic activity) did so only weakly compared with wild-type AKT1 (FIG. 15A; refs. 3, 4). These results were consistent with AKT1 kinase activity being necessary for asymmetric division.

Treating wild-type cancer cells with allosteric AKT inhibitors at low doses dramatically increases the frequency of both asymmetrically dividing and G0-like cells in HCT116 and MCF7 breast cancer cells (i.e., AKT1/2, MK2206; FIG. 15C; ref 1). These allosteric inhibitors are known to bind to the AKT1 pleckstrin homology domain, inducing conformational change and protein displacement from the cell membrane, thus promoting its ubiquitination and proteasome-mediated degradation (5). It was therefore hypothesized that asymmetric division might depend on targeted degradation of AKT1 protein.

TTC3 is a RING-type E3 protein-ligase known to ubiquitinate AKT1 at the lysine-8 and lysine-14 residues leading to its destruction by the proteasome (6). It was found that G0-like cells from wild-type MCF7 express high levels of TTC3 protein compared with proliferating cells, consistent with a potential role for this E3 ligase in producing AKT1low cells (data not shown). In addition, inducible shRNA knock-down of TTC3 suppressed the frequency of G0-like cells in both wild-type HCT116 and MCF7 (FIG. 15C). Furthermore, AKT1-K8R, AKT1-K14R, and AKT1-K8R/K14R double mutant proteins (which cannot be ubiquitinated by TTC3) failed to rescue the formation of G0-like cells in the AKT1/2/line (FIG. 15C-left). Moreover, two different small molecules that inhibit proteasome function reduced the frequency of G0-like cells in both wild-type HCT116 and MCF7 when used at doses that do not affect overall cell proliferation (i.e., MG-132, bortezomib; FIG. 15C). Overall, these results were consistent with enzymatically active AKT1 being ubiquitinated by TTC3 and degraded by the proteasome during cell division to produce slow proliferators.

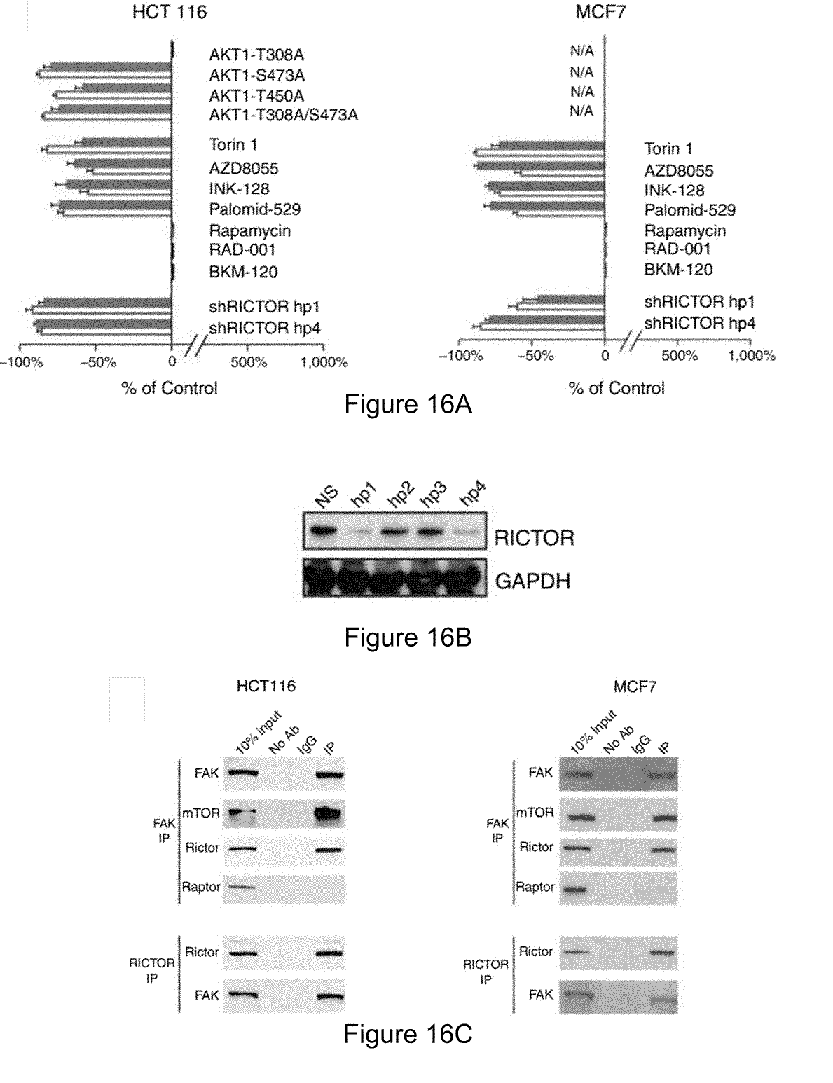

AKT1 is usually activated by two different upstream kinases: PDPK1 phosphorylates AKT1 at the T308 residue, whereas the mTORC2 kinase complex phosphorylates the AKT1-S473 and AKT1-T450 sites (7, 8). Similar to AKT1 cDNA, overexpression of the AKT1-T308A cDNA mutant (which cannot be phosphorylated by PDPK1) completely restored the production of asymmetrically dividing and G0-like cells in AKT1/2/cells (FIG. 16A, left). In contrast, AKT1-S473A, AKT1-T450A, and an AKT1-T308A/AKT1-S473A double mutant (all of which cannot be phosphorylated by mTORC2) did not produce phenotypic rescue in these cells (FIG. 6A, left). It was also found that four structurally different small molecules that inhibit both mTORC2 and mTORC1 signaling reduced the frequency of asymmetrically dividing and G0-like cells in both wild-type HCT116 and MCF7 cancer cells at low doses that did not appreciably inhibit cell proliferation (i.e., TORIN1, AZD8055, INK-128, Palomid-529; FIG. 16A). In contrast, the production of G0-like cells was not suppressed either by inhibitors that preferentially target the TORC1 signaling complex alone (i.e., rapamycin, RAD-001) or by a pan-class I PI3K inhibitor (i.e., BKM-120), when used at target-suppressing doses in these wild-type cells (FIG. 16A). In addition, inducible shRNA knockdown of RICTOR (an obligate member of the mTORC2 signaling complex) suppressed the production of both asymmetrically dividing and slowly proliferating G0-like cells in both wild-type HCT116 and MCF7 (FIGS. 16A and 16B). It was also found in asymmetrically dividing cells, that the slow proliferator daughter cells (i.e., H3K9me2low) were phospho-AKT1-S473high but phospho-AKT1-T308normal (data not shown). In contrast, after cytokinesis these slow proliferators (i.e., H3K9me2low) were AKTlow and commensurately phospho-AKT1-S4731ow and phospho-AKT1-T308low (data not shown; ref. 1). In aggregate, these results support a dynamic model whereby differential phosphorylation of AKT1 by mTORC2 precedes the production of slow proliferators with low levels of AKT1 protein.

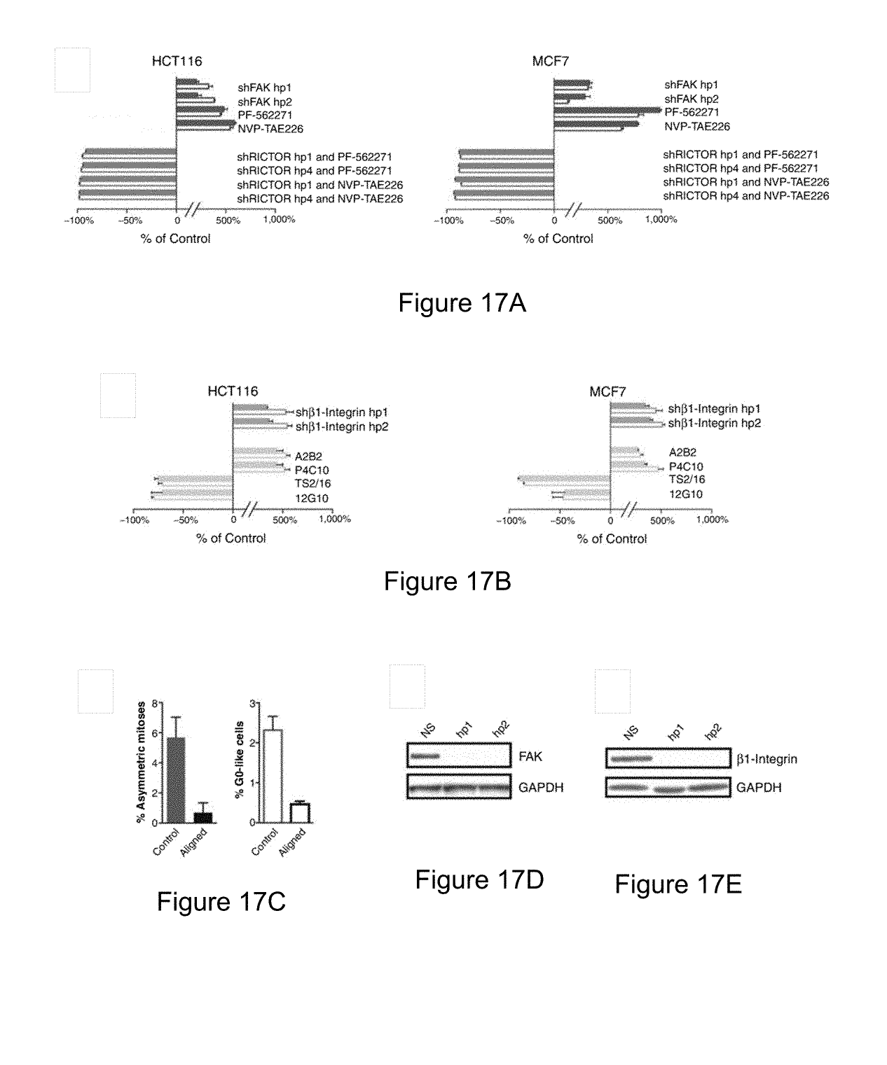

To identify an upstream regulator that might activate mTORC2 signaling during asymmetric division, an immunoprecipitation approach was used to identify proteins that physically interact with the mTORC2 complex during mitosis. HCT116 and MCF7 cells were treated with nocodazole, to synchronize cells in metaphase, and then whole-cell protein lysates prepared 2 hours after release of this synchronization with the cells still in mitosis. It was found that immunoprecipitation with a RICTOR antibody (under conditions that maintain integrity of the mTORC2 complex in whole-cell lysates) pulled down focal adhesion kinase (FAK) protein in both HCT116 and MCF7. Reciprocally, immunoprecipitation with a FAK antibody pulled down both mTOR kinase and RICTOR, but not RAPTOR (an obligate member of the related mTORC1 complex), confirming the specific interaction of FAK with mTORC2 complex in these cells (FIG. 16C). This observation suggested that FAK activity might regulate mTORC2-associated AKT1 degradation and asymmetric cancer cell division. Furthermore, inducible shRNA knockdown of FAK increased both asymmetrically dividing and G0-like cells in HCT116 and MCF7 (FIGS. 17A and 17D). Similarly, inhibiting FAK enzymatic activity with two different small molecules increased the frequency of both asymmetrically dividing and G0-like cells (i.e., PF-562271, NVP-TAE226; FIG. 17A). However, FAK inhibitors failed to increase asymmetries or slow proliferators after RICTOR knockdown (FIG. 17A). These findings were consistent with a model whereby a loss of FAK activity induces mTORC2-mediated asymmetric cancer cell division.

Integrins are a family of heterodimeric transmembrane receptors that transduce signals from the extracellular matrix, by activating signaling intermediaries, including FAK, to increase the cell cycle, survival, and motility of cancer and normal cells (9). It was therefore reasoned that decreased integrin signaling might be the proximate cause for a loss in FAK activity resulting in asymmetric mitosis. In fact, shRNA knockdown of β1-integrin (i.e., ITGB1) increased the fraction of asymmetrically dividing and G0-like cells in both HCT116 and MCF7 (FIGS. 17B and 17E). In addition, blocking β1-integrin function with two different monoclonal antibodies also increased both asymmetrically dividing and G0-like cells (i.e., A2B2, P4C10; FIG. 17B; ref. 10). However, activating β1-integrin signaling with two other monoclonal antibodies, which force β1-integrin into a constitutive “on” state by imposing a conformational change, eliminated both asymmetries and slow proliferators in these cell lines (i.e., TS2/16, 12G10; FIG. 17B; ref 10).

These observations indicated that the asymmetric cancer cell divisions can result from random variation in β1-integrin signaling related to extracellular irregularities within cell culture. Cancer cells were grown on engineered matrices displaying type-I collagen (a major extracellular matrix protein that activates β1-integrin) closely aligned in a regular fibrillar pattern, to assure uniform β1-integrin activation in any cancer cell undergoing mitosis (11). Notably, cancer cells proliferating in this structured collagen matrix did not produce asymmetries or G0-like cells, in contrast with typical cell culture (FIG. 17C). In the aggregate, these results were consistent with loss in β1-integrin signaling during mitosis (likely resulting from random irregularity in extracellular type-I collagen) triggering a conserved pathway to produce slow proliferators in vitro.

Discussion

The proliferative heterogeneity among cancer cells within tumors generally correlates with differences in growth, response to treatment, and disease relapse in patients with cancer (12). As described herein, cancer cells occasionally divide asymmetrically to spawn AKTlow, MCM2low, H3K9me2low, HES1high progeny that proliferate slowly and are resistant to cytotoxic chemotherapy in cell culture (1% of cell divisions; ref 1). The existence of these AKTlow cancer cells within actual human breast tumors where they appear to survive intensive, combination chemotherapy is also demonstrated herein, indicating that these cells can mediate clinically important chemoresistance (1). Described herein is a signaling pathway that is triggered in dividing cancer cells to spawn these slow proliferators in vitro. This pathway involves a decrease in β1-integrin/FAK activity, activation of the mTORC2 complex, and suppression of AKT1 protein levels through TTC3/proteasome-mediated degradation. Interestingly, any dividing cancer cell appears capable of triggering the β1-integrin pathway that is described herein to produce AKT1low slow proliferators. This facultative behavior presumably occurs if dividing cancer cells encounter irregularities in extracellular type I collagen, although additional cooperative factors yet to be discovered may also be required. Moreover, it is described herein that activation of β1-integrin signaling with monoclonal antibodies or inhibition of mTORC2 signaling with small molecules reduces asymmetric cancer cell division and the production of these slow proliferators. These findings permit avenues for experimentally or therapeutically manipulating and studying the production of AKT1low slow proliferators both in vitro and in vivo.

These results also offer useful molecular insight into different signaling molecules that are under intensive investigation as therapeutic targets for various cancer types, which may carry implications for the development and use of clinical inhibitors that target these important molecules. For example, the MCF7 and HCT116 cancer cells have activating mutations in PIK3CA, and are thus dependent on constitutive PI3K/AKT signaling for their proliferation and survival. Despite this dependency, however, it is described herein that these ER [symbol] breast and colorectal cancers retain the β1-integrin pathway that produces AKT1low slow proliferators. This indicates that cancer cells can actually derive some indispensible advantage from suppressing AKT1 to produce slow proliferators in this way. In addition, it is described herein that a quantitative reduction in β1-integrin, FAK, or AKT1 (rather than AKT2/3) signaling in cancer cells produces this reversible cell-cycle arrest through a conserved pathway, compared with complete suppression of these targets that generally results in cell death or senescence.

Our results also indicate that FAK can physically interact with and functionally suppress the mTORC2 signaling complex during cell division. Moreover, while mTORC2 activity is normally required for AKT1 activation, this multifunctional signaling complex is also necessary for triggering AKT1 degradation during asymmetric cancer cell division. Finally, TTC3-mediated proteasome degradation of AKT1 is necessary for the production of AKT1low slow proliferators.

### REFERENCES

1. Dey-Guha I, Wolfer A, Yeh A C, Albeck J G, Darp R, Leon E, et al. Asymmetric cancer cell division regulated by AKT. Proc Natl Acad Sci USA 2011; 108:12845-50.
2. Ericson K, Gan C, Cheong I, Rago C, Samuels Y, Velculescu V E, et al. Genetic inactivation of AKT1, AKT2, and PDPK1 in human colorectal cancer cells clarifies their roles in tumor growth regulation. Proc Natl Acad Sci USA 2010; 107:2598-603.
3. Okuzumi T, Fiedler D, Zhang C, Gray D C, Aizenstein B, Hoffman R, et al. Inhibitor hijacking of Akt activation. Nat Chem Biol 2009; 5: 484-93.
4. Aoki M, Batista O, Bellacosa A, Tsichlis P, Vogt P K. The akt kinase: molecular determinants of oncogenicity. Proc Natl Acad Sci USA 1998; 95:14950-5.
5. Jo H, Mondal S, Tan D, Nagata E, Takizawa S, Sharma A K, et al. Small molecule-induced cytosolic activation of protein kinase Akt rescues ischemia-elicited neuronal death. Proc Natl Acad Sci USA 2012; 109: 10581-6.
6. Suizu F, Hiramuki Y, Okumura F, Matsuda M, Okumura A J, Hirata N, et al. The E3 ligase TTC3 facilitates ubiquitination and degradation of phos-phorylated Akt. Dev Cell 2009; 17:800-10.
7. Sarbassov D D, Guertin D A, Ali S M, Sabatini D M. Phosphorylation and regulation of Akt/PKB by the rictor-mTOR complex. Science 2005; 307: 1098-101.
8. Manning B D, Cantley L C. AKT/PKB signaling: navigating downstream. Cell 2007; 129:1261-74.
9. Hynes R O. Integrins: bidirectional, allosteric signaling machines. Cell 2002; 110:673-87.
10. Byron A, Humphries J D, Askari J A, Craig S E, Mould A P, Humphries M J. Anti-integrin monoclonal antibodies. J Cell Sci 2009; 122(Pt 22): 4009-11.
11. Bessea L, Coulomb B, Lebreton-Decoster C, Giraud-Guille M M. Production of ordered collagen matrices for three-dimensional cell culture. Biomaterials 2002; 23:27-36.
12. Hong W K, Bast R C, Hait W N, Kufe D W, Pollock R E, Weichselbaum R R, et al. Cancer Medicine, 8th Edition. Shelton, Conn.: People's Medical Publishing House, 2010. xxv, 2021 pp.

What is claimed herein is:

1. A method of modulating the rate of asymmetric proliferation in a cancer cell, the method comprising:
contacting the cancer cell with a modulator of AKT1 degradation;
wherein an increase in AKT1 degradation increases the rate of asymmetric proliferation in the cancer cell; and
wherein a decrease in AKT1 degradation decreases the rate of asymmetric proliferation in the cancer cell.

2. The method of claim 1, wherein the modulator of AKT1 degradation is an agonist of AKT1 degradation selected from the group consisting of:
an allosteric inhibitor of AKT1; an allosteric inhibitor of AKT1/2; MK2206; an inhibitor of FAK; an inhibitor of β1-integrin; PF-562271; and NVP-TAE226.

3. The method of claim 2, whereby slow proliferator cancer cells are produced.

4. The method of claim 1, wherein the modulator of AKT1 degradation is an inhibitor of AKT1 degradation selected from the group consisting of:
inhibitors of mTORC2 signaling; inhibitors of mTORC2; TORIN1; AZD8055; INK128; Palomid-529; inhibitors of mTORC2 expression; inhibitors of RICTOR;
inhibitors of RICTOR expression; an inhibitor of TTC3; MG-132; bortezomib; an
inhibitor of ATK1 expression; an agonist of β1-integrin; and a cell medium comprising a fibrillar pattern of collagen.

5. A method of treating cancer in a subject in need thereof, the method comprising:
administering an inhibitor of AKT1 degradation to the subject.

6. The method of claim 5, wherein the method further comprises administering a cancer therapy that targets fast proliferator cancer cells.

7. The method of claim 6, wherein the inhibitor of AKT1 degradation is administered before the administration of a cancer therapy that targets fast proliferator cancer cells.

8. The method of claim 7, wherein the inhibitor of AKT1 degradation is administered at least 1 day before the administration of a cancer therapy that targets fast proliferator cancer cells.

9. The method of claim 7, wherein the inhibitor of AKT1 degradation is administered at least 3 days before the administration of a cancer therapy that targets fast proliferator cancer cells.

10. The method of claim 5, wherein the subject has been determined to have a subpopulation of cancer cells expressing increased levels of one or more genes selected from the group consisting of:
Hes1 and TTC3;
or decreased levels of one or more genes selected from the group consisting of:
AKT1; H3K9me2; MCM2; MK167; CDC6; GMNN; AURKA; PLK1; H3S10ph; H3K4me2; H3K9me2; H3K27me3; H4K12ac; and H4K16ac.
wherein an increased level is a level statistically significantly higher than the level of expression found in at least 70% of cancer cells obtained from the same tumor and a decreased level is a level statistically significantly lower than the level of expression found in at least 70% of cancer cells obtained from the same tumor.

11. The method of claim 10, wherein the subject has been determined to have cancer cells expressing increased levels of TTC3; and optionally,
increased levels of Hes1;
or decreased levels of one or more genes selected from the group consisting of: AKT1; H3K9me2; MCM2; MK167; CDC6; GMNN; AURKA; PLK1; H3S10ph; H3K4me2; H3K9me2; H3K27me3; H4K12ac; and H4K16ac.

12. The method of claim 10, wherein the subject has been determined to have cancer cells expressing increased levels of Hes1 and TTC3 and decreased levels AKT1; H3K9me2; and MCM2.

13. The method of claim 10, wherein the expression level of the one or more genes is the level of polypeptide expression product.

14. The method of claim 10, wherein the expression level is determined by immunochemistry.

15. A method comprising;
(i) obtaining a tumor biopsy from a subject;
(ii) determining the expression level of TTC3 in cells obtained from the subject;
(iii) identifying the presence of slow proliferators in the tumor when cells with increased levels of expression of TTC3 are detected.

16. The method of claim 15, wherein the expression level of TTC3 and optionally Hes1, AKT1; H3K9me2; MCM2; MK167; CDC6; GMNN; AURKA; PLK1; H3S10ph; H3K4me2; H3K9me2; H3K27me3; H4K12ac; or H4K16ac are determined; and
wherein the presence of slow proliferators in the tumor is indicated when cells with increased levels of expression of TTC3 and optionally,
increased levels of expression of Hes1 or decreased levels of expression of AKT1; H3K9me2; MCM2; MK167; CDC6; GMNN; AURKA; PLK1; H3S10ph; H3K4me2; H3K9me2; H3K27me3; H4K12ac; or H4K16ac are detected.

17. The method of claim 15, wherein the method further comprises administering an inhibitor of AKT1 degradation to the subject to a subject identified as having cancer cells with increased levels of expression of TTC3.

18. A method of producing slow proliferator cancer cells, the method comprising:
contacting a cancer cell with an agonist of AKT1 degradation;
(ii) maintaining the cancer cells treated in step (i).

19. The method of claim 18, wherein the method further comprises the step of enriching the cells of step (ii) for slow proliferators by selecting for cells having increased levels of expression of TTC3 and optionally, increased levels of expression of Hes1 or decreased levels of expression of AKT1; H3K9me2; MCM2; MK167; CDC6; GMNN; AURKA; PLK1; H3S10ph; H3K4me2; H3K9me2; H3K27me3; H4K12ac; or H4K16ac.

20. The method of claim 18, wherein the method further comprises the step of enriching the cells of step (ii) for slow proliferators by selecting for cells having increased levels of expression of TTC3 and Hes1 and decreased levels of expression of AKT1; H3K9me2; MCM2; MK167; CDC6; GMNN; AURKA; PLK1; H3S10ph; H3K4me2; H3K9me2; H3K27me3; H4K12ac; and H4K16ac.


SEQUENCE LISTING (2 sequences)

SEQ ID NO: 1 - DNA, 23 bp, Artificial Sequence
  Description of Artificial Sequence Synthetic primer
cccttgaacctcctcgttcgacc

SEQ ID NO: 2 - DNA, 23 bp, Artificial Sequence
  Description of Artificial Sequence Synthetic primer
ctgtttaccttcatcgtgcagag